(19) United States
(12) Patent Application Publication
(10) Pub. No.: US 2025/0154253 A1
(43) Pub. Date: May 15, 2025

(54) TARGETING CD47 WITH Fc-ENHANCED ACTIVITY

(71) Applicant: The Rockefeller University, New York, NY (US)

(72) Inventors: Jeffrey V. Ravetch, New York, NY (US); Juan Osorio, New York, NY (US)

(21) Appl. No.: 18/945,938

(22) Filed: Nov. 13, 2024

Related U.S. Application Data
(60) Provisional application No. 63/599,109, filed on Nov. 15, 2023.

(51) Int. Cl. C07K 16/28; A61K 39/00; A61P 35/00

(52) U.S. Cl. CPC C07K 16/2803; A61P 35/00; A61K 2039/505; C07K 2317/24

(57) ABSTRACT

This disclosure provides anti-CD47 antibodies or antigen-binding portions thereof comprising a modified Fc region that activates FcγRs and enhances in vivo antitumor activity. This disclosure further provides a humanized CD47/SIRPα/FcγR mouse model for evaluating anti-CD47 antibodies or antigen-binding portions thereof.

### CROSS-REFERENCE TO RELATED APPLICATIONS

This application claims the benefit under 35 U.S.C. § 119 (e) of the earlier filing date of U.S. Provisional Patent No. 63/599,109, filed on Nov. 15, 2023, which is hereby incorporated by reference in its entirety.

### STATEMENT REGARDING FEDERALLY SPONSORED RESEARCH

This invention was made with government support under grant numbers R35CA196620 and K08CA266740 awarded by the National Institutes of Health (NIH). The U.S. Government has certain rights in this invention.

### REFERENCE TO AN ELECTRONIC SEQUENCE LISTING

The contents of the electronic sequence listing (070413.20830SeqList.xml; Size: 53,971 bytes; and Date of Creation: Nov. 12, 2024) is herein incorporated by reference in its entirety.

### FIELD OF THE INVENTION

The present invention relates to Fc-engineered anti-CD47 antibodies or antigen-binding portions thereof with enhanced binding to activating FcγRs that enhances in vivo systemic antitumor immunity while minimizing toxicity. The present invention further relates to a mouse model humanized for CD47, SIRPα and FcγRs for evaluating human anti-CD47 antibodies or antigen-binding portions thereof.

### BACKGROUND OF THE INVENTION

CD47 is a cell surface receptor expressed on healthy cells and often overexpressed on cancer cells, which protects them from phagocytosis by interacting with SIRP (signal regulatory protein) «, an inhibitory immunoreceptor expressed on myeloid cells including macrophages, neutrophils and subsets of dendritic cells. In several preclinical studies, therapeutic antibodies that block the interaction between CD47 and SIRPα promote effective antitumor responses by enabling elimination of both hematologic and solid tumor cells by phagocytes, enhancing cross-presentation of CD8⁺ T cells by antigen presenting cells, and triggering adaptive immune responses. However, anti-CD47 antibodies have demonstrated limited clinical benefit in a variety of malignancies, highlighting the need to better understand the mechanisms behind the efficacy of these therapies. To this end, a critical question that remains to be answered is whether the antitumor activity of these antibodies relies solely on blocking the CD47/SIRPα interaction by the fragment antigen-binding (Fab) domain, or whether interactions between the fragment crystallizable (Fc) and Fc gamma receptors (FcγRs) also contribute to their antitumor activity. Currently, there is no consensus on the ideal Fc format that will maximize the therapeutic index of antibodies targeting CD47.

Consequently, there remains a need to understand the mechanisms behind the therapeutic activity of antibodies targeting CD47 to identify an Fc-optimized antibody (ab) targeting hCD47 for effective in vivo antitumor activity.

### SUMMARY OF THE INVENTION

In one aspect, provided is a Fc-optimized anti-CD47 antibodies or antigen-binding portions thereof that boost systemic antitumor immunity with minimal toxicity. In one aspect, provided is an isolated antibody or antigen-binding portion thereof that specifically binds to human CD47, wherein the antitumor activity of the antibody or antigen-binding portion thereof comprises an Fc region modified to enhance the antitumor activity of the antibody or antigen-binding portion thereof, and wherein the Fc region comprises at least one mutation that is G236A/A330L/1332E with respect to SEQ ID NO: 13. In one embodiment, the Fc region comprises a hIgG1 having G236A/A330L/1332E mutations as set forth in SEQ ID NO: 8.

In some embodiments, the antibody or antigen-binding portion thereof comprises 5F9 or MIAP301. In one embodiment, the antibody or antigen-binding portion thereof comprises a heavy chain variable region having the amino acid sequence of SEQ ID NO: 11 and a light chain variable region having the amino acid sequence of SEQ ID NO: 12.

In one embodiment, the antibody or antigen-binding portion thereof increases phagocytosis of cancer cells.

In one embodiment, the antibody or antigen-binding portion thereof blocks the interaction of CD47 with hSIRPα.

In one embodiment, the antibody or antigen-binding portion thereof is humanized.

In some embodiments, a nucleic acid comprises a sequence encoding the antibody or antigen-binding portion thereof described herein. In some embodiments, an expression vector comprises the nucleic acid described herein. In some embodiments, a host cell comprises the nucleic acid described herein.

In some embodiments, provided is a pharmaceutical formulation comprising the antibody or antigen-binding portion thereof described herein and optionally a pharmaceutically acceptable carrier.

In some embodiments, provided is a method of treating cancer, comprising administering to a subject in need thereof a therapeutically effective amount of the antibody or antigen-binding portion thereof described herein. In some embodiments, the method further comprises administering a second therapeutic agent. In some embodiments, the second therapeutic agent comprises, a checkpoint inhibitor or a tumor antigen inhibitor. In some embodiments, the cancer is a CD47 expressing cancer comprising colorectal cancer, lymphoma, lung cancer, melanoma, breast cancer, ovarian cancer, endometrial cancer, cervical cancer, bladder cancer, prostate cancer, kidney cancer, glioma or glioblastoma, gastric cancer, esophageal carcinoma, osteosarcoma, or head and neck cancer.

In one aspect, provided is a mouse model for evaluating an anti-CD47 antibody or antigen-binding portion thereof, wherein the mouse comprises a humanized CD47, a humanized SIRPα, and humanized FcγRs. In some embodiments, the mouse recapitulates the toxicity observed with a blocking anti-CD47 antibody or antigen-binding portion thereof in a clinical setting. In some embodiments, the toxicity comprises anemia or thrombocytopenia. In some embodiments, provided is a method of generating the mouse model described herein, comprising administering to the mouse a nucleic acid molecule comprising or encoding one or more CRISPR sgRNAs having a nucleotide sequence of SEQ ID NO: 1, SEQ ID NO: 2, SEQ ID NO: 3, or SEQ ID NO: 4. In some embodiments, the mouse is backcrossed with a hFcγR mouse to generate hCD47/hSIRPα/hFcγR mice.

In one aspect, provided is a method of boosting systemic antitumor immunity with minimal toxicity, comprising intratumorally administering to subject in need thereof a therapeutically effective amount of an isolated antibody or antigen-binding portion thereof that specifically binds to human CD47, wherein the antibody or antigen-binding portion thereof comprises an Fc region modified to enhance the antitumor activity of the antibody or antigen-binding portion thereof, and wherein the Fc region comprises at least one mutation that is G236A/A330L/1332E with respect to SEQ ID NO: 13.

### BRIEF DESCRIPTION OF THE FIGURES

FIGS. 1A, 1B, 1C, 1D, and 1E show that engagement of activating FcγRs enhances in vivo antitumor activity of antibodies blocking mCD47. FIG. 1A shows the average growth±standard error of the mean (SEM) of subcutaneous (sq.) MC38 tumors in WT (left) or FcR-γ chain^{-/-} mice (right), treated with Fc variants of anti-mCD47 ab (MIAP301) or control (50 ug IT, days 8,10,14 and 18), n=8/group. FIG. 1B shows the average number of lung metastases±(Min to Max) of WT mice inoculated IV with B16 tumor cells, treated with anti-mCD47 ab Fc variants (MIAP301) or control (20 mg/Kg intraperitoneal (IP), days 1,4,7 and 11), n=6/group. FIG. 1C depicts the average growth±SEM of sq. B16 tumors in WT mice, treated with MIAP301 Fc variants or control (50 ug IT, days 8, 10, 14 and 18), n=6/group. FIG. 1D shows the average growth±SEM of sq. B16 tumors in WT mice, treated with MIAP301 Fc variants or control (50 ug IT, days 8,10,14 and 18) in combination with anti-gp75 ab (TA99-mIgG2a, 200 ug IP days 8, 10, 14 and 18), n=8/group. FIG. 1E shows the average growth±SEM of sq. B16 tumors in WT mice, treated with MIAP301 Fc variants or control (50 ug IT, days 8,10,14 and 18) in combination with anti-PD-1 ab (αPD-1, RMP1-14-mIgG1-D265A, 200 ug IP days 8, 10, 14 and 18). n=6/group. Student t test between the indicated experimental arms were performed in all panels. (*p<0.05; **p<0.01; ***p<0.001; ** p<0.0001). See also FIGS. 2A-2F.

FIGS. 2A, 2B, 2C, 2D, 2E, and 2F depict the generation and validation of Fc variants for mouse and chimeric anti-CD47 antibodies. FIG. 2A shows the binding profiles to mouse FcγRs of Fc variants for anti-CD47 antibodies (MIAP301, chimeric-5F9 and chimeric-2D3), activating-to-inhibitory binding ratio (A/I) to mFcγRs is shown. FIG. 2B shows the levels of expression of CD47 on different mouse cell lines, mean fluorescence intensity (MFI) is expressed on the right. FIG. 2C is a diagram showing the structure of the mouse antibody MIAP301 and binding of their Fc variants to recombinant mouse CD47 by ELISA. FIG. 2D is a diagram showing the structure of the chimeric antibody chimeric-5F9 and binding of their Fc variants to recombinant human CD47 by ELISA. FIG. 2E shows the binding of non-blocking (Chimeric 2D3-mIgG2a) and blocking (Chimeric SF9-mIgG2a) CD47 antibodies to recombinant human CD47 by ELISA. FIG. 2F is a competitive ELISA showing the capacity of blocking (mIgG2a Chimeric-SF9) and non-blocking (mIgG2a Chimeric-2D3) antibodies to disrupt the binding of recombinant hSIRPα to hCD47. One-Way ANOVA test was performed at the indicated concentration (FIGS. 1E and 1F). (ns, p>0.05; *p<0.05; ** p<0.01; ***p<0.001; ****p<0.0001). FIGS. 2A-2C are related to FIGS. 1A-1C. FIGS. 2D-2F are related to FIGS. 7A-7E.

FIGS. 3A, 3B, 3C, 3D, 3E, and 3F illustrate that anti-mCD47 ab Fc-enhanced for activating FcγRs correlates with increased number of tumors infiltrating macrophages and enhanced phagocytic activity. FIG. 3A depicts established MC38 tumors that were analyzed by flow cytometry 24 hours after three treatments with Fc variants of anti-mCD47 ab (MIAP301). Quantification of myeloid cell populations is shown, n=5-12/group. FIG. 3B shows representative images (10×) of MC38 tumors treated with Fc variants of anti-mCD47 ab (MIAP301) and assessed for F4/80 and CD11b staining with immunohistochemistry (Left). Dashed circles indicate tumor core (regions of interest, ROI). FIG. 3C shows the quantification of the percent of positive staining for each marker within the ROI (right), n=5/group. FIG. 3D shows the average growth+SEM of sq. MC38 tumors that were pre-treated with clodronate- or control-liposomes (100 uL, days 4, 7), then treated with MIAP301-mIgG2a or isotype control (50 ug IT, days 8, 10, 14 and 18) n=5/group. FIG. 3E shows the average growth+SEM of sq. MC38 tumors pre-treated with anti-CCR2 ab (αCCR2, MC-21) or isotype control (100 ug, days 7), then treatment with MIAP301-mIgG2a or isotype control was given (50 ug IT, days 8, 10, 14 and 18) n=5/group. FIG. 3F illustrates bone marrow derived macrophages that were obtained from WT mice were cocultured with CFSE-labeled MC38 or B16 tumor cells in the presence of PBS (Control) or Fc-modified anti-mCD47 ab (MIAP301) n=2-3/group. The percentage of phagocytosis was determined by the percentage of CFSE+ cells within the macrophage cell gate (CD11b+F480+). Analysis of fold changes compared to control is shown. Student t test between the indicated experimental arms were performed in all panels. Bars indicate the median with 95% CI (confidence interval) (Panel A and B), or the mean+SD (Panel E). (ns, p>0.05; *p<0.05; **p<0.01; *** p<0.001; ****p<0.0001). See also FIGS. 4A-4J and FIGS. 5A-5I.

FIGS. 4A, 4B, 4C, 4D, 4E, 4F, 4G, 4H, 4I, and 4J show changes in myeloid and T cells after treatment with Fc variants of anti-mCD47 ab and assessment of depletion of monocytes and macrophages. FIGS. 4A, 4B, 4C, and 4D depict established MC38 tumors that were analyzed by flow cytometry 24 hours after 3 treatments with Fc variants for CD47 antibodies (MIAP301) n=5-12/group. Quantification of FIG. 4A: CD45, FIG. 4B: CD4 and FIG. 4C: CD8 cells are shown. FIG. 4D illustrates that CD8″ T cells were also assessed for proliferation by Ki67 and markers of exhaustion (TIM3 and PD-1). FIG. 4E are representative flow cytometry plots of macrophages (Top), and monocytes (bottom) 24 h after treatment (×3) with MIAP301 Fc variants. FIG. 4F shows the Percentage of MHCII, CD80, CD86, PD-L1, CD163 and CD206 positive cells of total macrophages from MC38 tumors 24 hours after treatment (×3) with Fc variants of anti-mCD47 abs (MIAP301), FIG. 4G shows the peripheral blood levels of monocytes 24 hours after treatment with anti-CCR2 ab (Clone MC-21-mIgG2a, 100 ug) or isotype control in WT mice, n=15/group. FIG. 4H shows the quantification of intratumoral macrophages from MC38 tumors pre-treated with control- or clodronate-liposomes intravenously (IV) or intratumorally (IT) (100 uL, d.-3,-1) by flow cytometry, n=3/group. FIG. 4I shows the quantification of intratumoral monocytes from MC38 tumors pre-treated with control- or clodronate-liposomes intravenously (IV) or intratumorally (IT) (100 uL, d.-3,-1) by flow cytometry, n=3/group. FIG. 4J shows markers of activation and phenotype of BMDM co-cultured with MC38 tumors 2 hours after treatment with Fc subclasses of anti-mCD47 antibodies analyzed by flow cytometry n=3/group. Student t test between the indicated experimental arms were performed in all panels. Bars indicate the median with 95% CI (Panel A-I), or the mean+SD (Panel J). (ns, p>0.05; *p<0.05; **p<0.01; ***p<0.001; ****p<0.0001). FIGS. 4A and 4E-4J are related to FIGS. 3A-3F. FIGS. 4B-4D are related to FIGS. 6A-6E.

FIGS. 5A, 5B, 5C, 5D, 5E, 5F, 5G, 5H, and 5I demonstrate that Fc-enhanced anti-CD47 antibodies enhance in vitro antibody dependent cellular phagocytosis (ADCP) across different species-match systems. FIG. 5A shows representative flow cytometry plots of the percentage of phagocytosis of B16 cancer cells by bone marrow derived macrophages (BMDM) from WT mice after treatment with control or Fe subclasses of MIAP301. FIG. 5B shows the phagocytosis of Lewis lung carcinoma (LLC) or HKP1 (Kras G12D p53) lung adenocarcinoma after treatment with control or Fc subclasses of MIAP301, n=3/group. FIG. 5C depicts a representative flow cytometry plot and FIG. 5D shows the phagocytosis of MC38 hCD47 KI or B16 hCD47 KI by BMDM from hCD47/hSIRPα KI mice after treatment with control or Fe variants of Chimeric-5F9 (mFc, hFab). n=3/group. FIG. 5E shows the phagocytosis of MC38 hCD47 KI cells by BMDM from hCD47/hSIRPα KI mice pre-treated with different polarization conditions, n=2-6/group. FIG. 5F shows the phagocytosis of MC38 hCD47 KI or B16 hCD47 KI cells by BMDM from hCD47/hSIRPα KI mice after treatment isotype control, blocking (5F9), non-blocking (2D3) antibodies targeting hCD47, both in mIgG2a Fc format, n=2-3/group. FUG. 5G (Left) shows representative flow cytometry plots and (Right) phagocytosis of B16 hCD47 KI mice by BMDM from hCD47/hSIRPα/hFcγR mice after treatment with Fc variants of fully humanized anti-CD47 ab (5F9, hFc, and hFab). Macrophages were pre-treated with different polarization conditions as indicated. n=3-12/group. FIG. 5H (Left) shows representative flow cytometry plots and (right) phagocytosis of human cancer cell line Jurkat and FIG. 5I illustrates Raji by human monocyte derived macrophages (hMDM) after treatment with Fc variants of fully humanized 5F9, n=8/group. Student t test between the indicated experimental arms were performed in all panels. Bars indicate the mean±SD. (ns, p>0.05; *p<0.05; **p<0.01; ***p<0.001; ****p<0.0001). FIGS. 5A-5B are related to FIGS. 3A-3F. FIGS. 5C-5F are related to FIGS. 7A-7E. FIGS. 5G-5I are related to FIGS. 9A-9F.

FIGS. 6A, 6B, 6C, 6D, and 6E show that Fc-optimized anti-mCD47 antibodies decrease Tregs and promote infiltration of antigen-specific CD8+ T cells. FIG. 6A illustrates levels of expression of CD47 on T cell subsets isolated from MC38 tumors (n=7). FIG. 6B shows the percentage and absolute number of CD4+FOXP3+ Tregs from MC38 tumors 24 hours after three treatment with Fc variants of anti-mCD47 antibodies (MIAP301), n=10-18/group. FIG. 6C shows the percentage of granzyme B+ and tetramer+ (KSPWFTTL) cells of total CD8+ T cells from MC38 tumors after treatment with Fc variants of anti-mCD47 antibodies (MIAP301) n=8-12/group. FIG. 6D shows the average growth+SEM of sq. MC38 tumors that were pre-treated with anti-CD8 ab (αCD8, 2.43) or isotype control (100 ug. days 7, 12, 17), then treated with MIAP301-mIgG2a or isotype control (50 ug IT, days 8, 10, 14 and 18) n=5-7/group. FIG. 6E depicts MC38 tumors that were treated with MIAP301-mIg2a (50 ug every 3 days) until rejection was achieved. Then mice were rechallenged with 5 times more cells (10 million) at day 90 (top). Average growth+SEM of sq. MC38 tumors in mice that achieved rejection 90 days before (rechallenged grouped) or in and mice without prior implantation of MC38 tumors (naïve group) n=5/group (bottom). Student t test between the indicated experimental arms were performed in all panels. Quantification (left) and representative flow cytometry plots (right) are shown (panels A-C). Bars indicate the median with 95% CI (Panel A-C). (ns, p>0.05; *p<0.05; **p<0.01; ***p<0.001; ****p<0.0001). See also FIGS. 4A-4J.

FIGS. 7A, 7B, 7C, 7D, and 7E show the characterization of hCD47/hSIRPα KI mice and antitumor activity of blocking and non-blocking hCD47 antibodies Fc enhanced for activating FcγRs. FIG. 7A depicts representative flow cytometry panels of hCD47 and hSIRPα expression in RBC, platelets, CD3+ and CD11b+ leucocytes isolated from peripheral blood of human (top) and hCD47/hSIRPα KI mice (bottom). FIG. 7B shows the average growth+SEM of sq. MC38 hCD47 KI tumors in hCD47/hSIRPα KI mice, treated with chimeric anti-hCD47 ab Fc variants (chimeric-5F9) or isotype control (50 ug IT, days 8,10,14 and 18) n=5/group. FIG. 7C shows the number of lung metastases of hCD47/hSIRPα KI mice inoculated IV with B16 hCD47 KI tumor cells, treated with chimeric anti-hCD47 ab Fc variants (chimeric-5F9) or isotype control (20 mg/Kg IP, days 1,4,7 and 11) n=10-12/group. FIG. 7D is a schematic drawing showing the two ab formats proposed for effective antitumor activity of anti-CD47 antibodies. An ab blocking the interaction between CD47 and SIRPα and an Fc domain engaging activating FcγRs (left). A non-blocking anti-CD47 ab AND an Fc domain engaging activating FcγRs (right). FIG. 7E shows the lung metastases of hCD47/hSIRPα KI mice inoculated IV with B16 hCD47 KI tumor cells, treated with blocking (5F9-mIgG2a) and non-blocking (2D3-mIgG2a) chimeric anti-hCD47 antibodies or isotype control (20 mg/Kg IP, days 1, 4, 7 and 11) n=10-12/group. Student t test between the indicated experimental arms were performed in all panels. Bars indicate the median with 95% CI (Panel C and E). (*p<0.05; **p<0.01; ***p<0.001). See also FIGS. 2A-2F, 5A-5I, and 10A-10F.

FIGS. 8A, 8B, 8C, 8D, and 8E show that the humanized mouse model for CD47, SIRPα, and FcγR recapitulates activity and toxicity profile of fully humanized anti-CD47 antibodies. FIG. 8A is a schematic drawing showing the generation of the hCD47/hSIRPα/hFcγR mice. FIG. 8B shows the binding profiles to hFcγRs of Fc variants for anti-hCD47 antibodies (5F9). FIG. 8C shows the peripheral RBC and platelet count from WT mice after treatment with 5F9-hIgG4 and 5F9-GAALIE (5 mg/Kg days 0, 4, 8 and 11, left), mortality rate during systemic administration is shown (right), n=3-4/group. FIG. 8D shows the peripheral RBC and platelet count from hCD47/hSIRPα/hFcγR mice after treatment with increasing doses of 5F9-hIgG4 or as shown in FIG. 8E, 5F9-GAALIE ab (2.5, 5, and 10 mg/Kg days 0, 4, 8 and 11, left), mortality rate during systemic administration is shown (right), n=3-8/group. One-Way ANOVA test between the experimental arms was performed at day 4 and 11. Points indicate mean+SD (FIGS. 8C, 8D, and 8E). (ns, p>0.05; *p<0.05; **p<0.01; ***p<0.001; ****p<0.0001).

FIGS. 9A, 9B, 9C, 9D, 9E, and 9F illustrate that Fc-optimized humanized anti-CD47 ab promotes effective in vivo antitumor activity. FIG. 9A shows the average growth+SEM of injected sq. MC38 hCD47 KI tumors in hCD47/hSIRPα/hFcγR, treated with 5F9-hIgG4, 5F9-GAALIE ab or control (2.5 mg/Kg IT, days 7, 9, and 12). FIG. 9B shows the average growth+SEM of non-injected contralateral sq. MC38 hCD47 KI tumors in hCD47/hSIRPα/hFcγR mice, treated with 5F9-hIgG4, 5F9-GAALIE ab or control (2.5 mg/Kg IT, days 7, 9, and 12). FIG. 9C shows the average growth+SEM of total (injected and non-injected) sq. MC38 hCD47 KI tumors in hCD47/hSIRPα/hFcγR, treated with 5F9-hIgG4, 5F9-GAALIE ab or control (2.5 mg/Kg IT, days 7, 9, and 12). n=8/group. FIG. 9D depicts serial measurements of peripheral RBC and platelets from hCD47/hSIRPα/hFcγR mice after treatment with 5F9-hIgG4 or 5F9-GAALIE (2.5 mg/Kg IT, day 0 indicates the day when treatment was started). n=8/group. FIG. 9E shows the average growth+SEM of sq. B16 hCD47 KI tumors in hCD47/hSIRPα/hFcγR, treated with 5F9-hIgG4, 5F9-GAALIE ab or control (2.5 mg/Kg IT, days 8,10,14 and 18). n=8/group. FIG. 9F shows the average growth+SEM of sq. B16 hCD47 KI tumors in hCD47/hSIRPα/hFcγR, treated with 5F9-hIgG4, 5F9-GAALIE ab alone (2.5 mg/Kg IT, days 8, 10, 14 and 18), or in combination with anti-PD1 ab ((αPD-1, RMP1-14, 200 ug IP days 8, 10, 14 and 18), or control n=8/group. Student t test between the indicated experimental arms were performed in all panels. (*p<0.05; **p<0.01; ***p<0.001; ****p<0.0001). See also FIGS. 5A-5I.

FIGS. 10A, 10B, 10C, 10D, 10E, and 10F show the development of humanized mouse model for CD47 and SIRPα (hCD47/hSIRPα KI mice) and cancer cell lines. FIG. 10A is a schematic drawing showing CRISPR/Cas9-mediated gene-targeting strategy to generate CD47 (top) and SIRPα (bottom) KI mice. The double-strand break induced by the CRISPR/Cas9 in the flanking regions of the extracellular domain (ECD) of mCD47 and mSIRPα was replaced by their human counterparts via non-homologous end joining. FIG. 10B shows the expression of hCD47 (left) and hSIRPα (right) in RBC, platelets, CD3+ and CD11b+ leukocytes isolated from peripheral blood from human and hCD47/hSIRPα KI mice. Normalized levels calculated as median fluorescence intensity (MFI) stained sample/MFI isotype control, n=3/group. FIG. 10C depicts the complete cell blood count of WT and hCD47/hSIRPα KI mice n=5/group. FIG. 10D illustrates that recombinant hSIRPα was incubated with RBCs from WT and hCD47/hSIRPα KI mice and binding was only detected in RBCs from the hCD47/hSIRPα mouse (left). Recombinant hCD47 was incubated with leukocytes from WT and hCD47/hSIRPα KI mice, binding was primarily detected in leukocytes from the hCD47/hSIRPα mouse (right). FIG. 10E is flow cytometry analysis of expression of hCD47 and mCD47 in unstained, MC38 WT and MC38 hCD47 KI cells. FIG. 10F is flow cytometry analysis of expression of hCD47 and mCD47 in unstained, B16 WT and B16 hCD47 KI cells. Student t test between the indicated experimental arms were performed in all panels. Bars indicate the mean+SD. (ns, p>0.05; *p<0.05; **p<0.01; ***p<0.001; ****p<0.0001). FIGS. 10A-10F are related to FIGS. 7A-7E.

FIG. 11 is a schematic of Fc-optimized anti-CD47 antibodies.

### DETAILED DESCRIPTION OF THE INVENTION

### Anti-CD47 Antibodies

Provided herein are anti-CD47 antibodies or antigen-binding portions thereof that have desirable properties for use as therapeutic agents in treating diseases such as cancers. These properties include one or more of the ability to bind to human CD47 with high affinity, acceptably low immunogenicity in human subjects, and the ability to bind to activating FcγRs. In particular, the disclosed anti-CD47 antibodies or antigen-binding portions thereof are Fc-optimized to enhance in vivo systemic antitumor immunity. Preferably, the disclosed antibodies or antigen-binding portions thereof bind to human CD47 (SEQ ID NO: 7).

Examples of blocking anti-CD47 antibodies include, but are not limited to MIAP301 (Thermo Fisher) and 5F9 (U.S. Pat. No. 9,382,320B2 and Liu J, et al. PLOS ONE. 2015. 10 (9): e0137345. doi: 10.1371/journal.pone.0137345), or other CD47 blocking antibodies or SIRP-alpha fusion proteins currently used in clinical trials including those shown in Table 1.

TABLE 1
Representative anti-CD47 blocking antibodies
and SIRP-alpha fusion proteins.

| Compound | Fc format |
|---|---|
| 5F9 | hIgG4 (U.S. Pat. No. 9,382,320B2) |
| TTI-621 | hIgG1 (SIRPα fusion protein) |
| TTI-622 | hIgG4 (SIRPα fusion protein) |
| ALX 148 (Evorpacept) | Inert hIgG1 (SIRPα fusion protein) |
| SRF231 | hIgG4 |
| AO-176 | hIgG2 |
| IBI-188 | hIgG4 |
| IMC-002 | hIgG4 |
| STI-6643 | hIgG4 |
| AK117 | hIgG4 |
| HMPL-A83 | hIgG4 |

### Fragment

In some embodiments, an antibody provided herein is an antibody fragment. Antibody fragments include, but are not limited to, Fab, Fab′, Fab′-SH, F(ab′)2, Fv, and single-chain Fv (scFv) fragments, and other fragments described below, e.g., diabodies, triabodies tetrabodies, and single-domain antibodies. For a review of certain antibody fragments, see Hudson et al., Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life (U.S. Pat. No. 5,869,046).

Diabodies are antibody fragments with two antigen-binding sites that may be bivalent or bispecific (EP 404,097; WO 1993/01161; Hudson et al., Nat. Med. 2003. 9:129-134; and Hollinger et al., PNAS. 1993. 90:6444-6448). Triabodies and tetrabodies are also described in Hudson et al., Nat. Med. 2003. 9:129-134.

Single-domain antibodies are antibody fragments comprising all or a portion of the heavy chain variable domain or all or a portion of the light chain variable domain of an antibody. In some embodiments, a single-domain antibody is a human single-domain antibody (DOMANTIS, Inc., Waltham, Mass.; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### Chimeric and Humanized Antibodies

In some embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding portions thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (Sims et al. J. Immunol. 1994.151:2296); framework regions derived from the consensus sequence of human antibodies of a particular subgroup of light or heavy chain variable regions (Carter et al. PNAS. 1992. 89:4285; and Presta et al. J. Immunol. 1993. 151:2623); human mature (somatically mutated) framework regions or human germline framework regions (Almagro and Fransson, Front. Biosci. 2008. 13:1619-1633); and framework regions derived from screening FR libraries (Baca et al., J. Biol. Chem. 1997. 272:10678-10684 and Rosok et al., J. Biol. Chem. 1996. 271:22611-22618).

### Human Antibodies

In some embodiments, an antibody provided herein is a human antibody or antigen-binding portion thereof. Human antibodies can be produced using various techniques known in the art or using techniques described herein. Human antibodies are described generally in van Dijk and van de Winkel, Curr. Opin. Pharmacol. 5:368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE® technology; U.S. Pat. No. 5,770,429 describing HUMAB technology; U.S. Pat. No. 7,041,870 describing K-M MOUSE technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibodies or antigen-binding portions thereof of the present disclosure may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., in Methods in Molecular Biology 178:1-37 (O'Brien et al., ed., Human Press, Totowa, N.J., 2001) and further described, e.g., in the McCafferty et al., Nature. 1990. 348:552-554; Clackson et al., Nature. 1991. 352:624-628; Marks et al., J. Mol. Biol. 1992. 222:581-597; Marks and Bradbury, in Methods in Molecular Biology 248:161-175 (Lo, ed., Human Press, Totowa, N.J., 2003); Sidhu et al., J. Mol. Biol. 2004.338 (2): 299-310; Lee et al., J. Mol. Biol. 340 (5): 1073-1093 (2004); Fellouse, PNAS. 2004. 101 (34): 12467-12472; and Lee et al., J. Immunol. Methods. 2004. 284 (1-2): 119-132.

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol. 1994.12:433-455. Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J. 1993 12:725-734. Finally, naive libraries can also be made synthetically by cloning unrearranged V-gene segments from stem cells and using PCR primers containing random sequences to encode the highly variable CDR3 regions and to accomplish rearrangement in vitro, as described by Hoogenboom and Winter, J. Mol. Biol. 1992. 227:381-388. Patent publications describing human antibody phage libraries include, for example, U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Antibodies or antibody fragments isolated from human antibody libraries are considered human antibodies or human antibody fragments herein.

### Variants

In some embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen binding.

### Substitution, Insertion, and Deletion Variants

In some embodiments, provided are antibody (or antigen-binding portion thereof) variants having one or more amino acid substitutions. Sites of interest for substitutional mutagenesis include the HVRs and FRs. Conservative substitutions are defined herein. Amino acid substitutions may be introduced into an antibody of interest and the products screened for a desired activity, e.g., retained/improved antigen binding, decreased immunogenicity, or improved antibody-dependent cell-mediated cytotoxicity (ADCC) and complement-dependent cytotoxicity (CDC).

Accordingly, an antibody or antigen-binding portion thereof described herein can comprise one or more conservative modifications of the CDRs, heavy chain variable region, or light variable regions described herein. A conservative modification or functional equivalent of a peptide, polypeptide, or protein disclosed herein refers to a polypeptide derivative of the peptide, polypeptide, or protein, e.g., a protein having one or more point mutations, insertions, deletions, truncations, a fusion protein, or a combination thereof. It substantially retains the activity to of the parent peptide, polypeptide, or protein (such as those disclosed herein). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent. Accordingly, within the scope of this disclosure are heavy chain variable region or light variable regions having one or more point mutations, insertions, deletions, truncations, a fusion protein, or a combination thereof, as well as antibodies having the variant regions.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the disclosure. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used (ncbi.nlm.nih.gov).

As used herein, the term “conservative modifications” refers to amino acid modifications that do not significantly affect or alter the binding characteristics of the antibody containing the amino acid sequence. Such conservative modifications include amino acid substitutions, additions and deletions. Modifications can be introduced into an antibody or antigen-binding portion described herein by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: (i) amino acids with basic side chains (e.g., lysine, arginine, histidine), (ii) acidic side chains (e.g., aspartic acid, glutamic acid), (iii) uncharged polar side chains (e.g., glycine, asparagine, glutamine, serine, threonine, tyrosine, cysteine, tryptophan), (iv) nonpolar side chains (e.g., alanine, valine, leucine, isoleucine, proline, phenylalanine, methionine), (v) beta-branched side chains (e.g., threonine, valine, isoleucine), and (vi) aromatic side chains (e.g., tyrosine, phenylalanine, tryptophan, histidine).

Non-conservative substitutions will entail exchanging a member of one of these classes for another class.

An exemplary substitutional variant is an affinity matured antibody, which may be conveniently generated, e.g., using phage display-based affinity maturation techniques such as those described in, e.g., Hoogenboom et al., in Methods in Molecular Biology 178:1-37 (O'Brien et al., ed., Human Press, Totowa, N.J., (2001). Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Fc Region Variants

The variable regions of the antibody described herein can be linked (e.g., covalently linked or fused) to an Fc, e.g., an IgGl, IgG2, IgG3 or IgG4 Fc, which may be of any allotype or isoallotype, e.g., for IgGI: Glm, Glml (a), Glm2 (x), Glm3 (f), Glml7 (z); for IgG2: G2m, G2m23 (n); for IgG3: G3m, G3m21 (gl), G3m28 (g5), G3 ml1 (b0), G3m5 (bl), G3ml3 (b3), G3ml4 (b4), G3ml0 (b5), G3ml5 (s), G3ml6 (t), G3m6 (c3), G3m24 (c5), G3m26 (u), G3m27 (v); and for K: Km, Km1, Km2, Km3 (Jefferies et al. mAbs. 2009. 1:1). In some embodiments, the antibodies variable regions described herein are linked to an Fc that binds to one or more activating Fc receptors (such as FcγI, Fcγlla, or FcγIIIa), and thereby stimulate ADCC and may cause T cell depletion. In some embodiments, the antibody variable regions described herein are linked to an Fc that causes depletion.

In some embodiments, the antibody variable regions described herein may be linked to an Fc comprising one or more modifications, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, to alter one or more functional properties of the antibody. The numbering of residues in the Fc region is that of the EU index of Kabat.

The Fc region encompasses domains derived from the constant region of an immunoglobulin, preferably a human immunoglobulin, including a fragment, analog, variant, mutant, or derivative of the constant region. Suitable immunoglobulins include IgGl, IgG2, IgG3, IgG4, and other classes such as IgA, IgD, IgE, and IgM. The constant region of an immunoglobulin is defined as a naturally-occurring or synthetically-produced polypeptide homologous to the immunoglobulin C-terminal region, and can include a CHI domain, a hinge, a CH2 domain, a CH3 domain, or a CH4 domain, separately or in combination. In some embodiments, an antibody or antigen-binding portion described herein has an Fc region other than that of a wild type IgA1. The antibody can have an Fc region from that of IgG (e.g., IgG1, IgG2, IgG3, and IgG4) or other classes such as IgA2, IgD, IgE, and IgM. The Fc can be a mutant form of IgA1.

The constant region of an immunoglobulin is responsible for many important antibody functions, including Fc receptor (FcR) binding and complement fixation. There are five major classes of heavy chain constant region, classified as IgA, IgG, IgD, IgE, IgM, each with characteristic effector functions designated by isotype. For example, IgG is separated into four subclasses known as IgGl, IgG2, IgG3, and IgG4.

Ig molecules interact with multiple classes of cellular receptors. For example, IgG molecules interact with three classes of Fcγ receptors (FcγR) specific for the IgG class of antibody, namely FcγRI, FcγRII, and FcγRIIL. The important sequences for the binding of IgG to the FcγR receptors have been reported to be located in the CH2 and CH3 domains. The serum half-life of an antibody is influenced by the ability of that antibody to bind to an FcR.

In some embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, one may make modifications in the Fc region in order to generate an Fc variant that (a) has increased or decreased ADCC, (b) increased or decreased CDC, (c) has increased or decreased affinity for Clq and/or (d) has increased or decreased affinity for an Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc., substitutions therein, e.g., of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli, such as proline iminopeptidase. In other embodiments, one or more glycosylation sites within the Fc domain may be removed. Residues that are typically glycosylated (e.g., asparagine) may confer cytolytic response. Such residues may be deleted or substituted with unglycosylated residues (e.g., alanine).

In one embodiment, the hinge region of Fc is modified such that the number of cysteine residues in the hinge region is altered, e.g., increased or decreased. This approach is described further in U.S. Pat. No. 5,677,425 by Bodmer et al. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

Antibodies or antigen-binding portions thereof described herein may comprise the variable domains provided herein combined with constant domains comprising different Fc regions, selected based on the biological activities (if any) of the antibody for the intended use (Salfeld. Nat, Biotechnol. 2007. 25:1369), Human IgGs, for example, can be classified into four subclasses, IgG1, IgG2, IgG3, and IgG4, and each of these comprises an Fc region having a unique profile for binding to one or more of Fcγ receptors (activating receptors FcγRI (CD64), FcγRIIA, FcγRIIC (CD32a,c); FcγRIIIA and FcγRIIIB (CD16a,b) and inhibiting receptor FcγRIIB (CD32b), and for the first component of complement (C1q). Human IgG1 and IgG3 bind to all Fcγ receptors; IgG2 binds to FcγRIIA_{H131}, and with lower affinity to FcγRIIA_{R131} FcγRIII_{AV158}; IgG4 binds to FcγRI, FcγRIIA, FcγRIIB, FcγRIIC, and FcγRIIIA_{V158}; and the inhibitory receptor FcγRIIB has a lower affinity for IgG1, IgG2 and IgG3 than all other Fcγ receptors (Bruhns et al. Blood. 2009. 113:3716). Studies have shown that FcγRI does not bind to IgG2, and FcγRIIIB does not bind to IgG2 or IgG4. Id. In general, with regard to ADCC activity, human IgG1≥ IgG3>>IgG4≥IgG2. As a consequence, for example, an IgG1 constant domain, rather than an IgG2 or IgG4, might be chosen for use in a drug where ADCC is desired; IgG3 might be chosen if activation of FcγRIIIA-expressing NK cells, monocytes of macrophages; and IgG4 might be chosen if the antibody is to be used to desensitize allergy patients. IgG4 may also be selected if it is desired that the antibody lack all effector function.

Anti-human CD47 variable regions described herein may be linked (e.g., covalently linked or fused) to an Fc, e.g., an IgG1, IgG2, IgG3 or IgG4 Fc, which may be of any allotype or isoallotype, e.g., for IgG1: G1m, G1 ml (a), G1m2 (x), G1m3 (f), G1m17 (z); for IgG2: G2m, G2m23 (n); for IgG3: G3m, G3m21 (g1), G3m28 (g5), G3m11 (b0), G3m5 (b1), G3m13 (b3), G3m14 (b4), G3m10 (b5), G3m15 (s), G3m16 (t), G3m6 (c3), G3m24 (c5), G3m26 (u), G3m27 (v) (Jefferis et al. mAbs. 2009. 1:1). Selection of allotype may be influenced by the potential immunogenicity concerns, e.g. to minimize the formation of anti-drug antibodies.

In some embodiments, anti-CD47 antibodies or antigen-binding portions thereof described herein have an Fc that binds to or has enhanced binding to activating FcγRs. This interaction in turn promotes tumor infiltration of macrophages and antigen-specific T cells, while depleting regulatory T cells. In some embodiments, the Fc region comprises at least one mutation. In one embodiment, the at least one mutation is G236A/A330L/1332E (GAALIE) with respect to SEQ ID NO: 13.

### Multivalent Antibodies

In one embodiment, the antibodies or antigen-binding portions thereof described herein may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen) (U.S.P.N. 2009/0130105). In each case, at least one of the binding sites will comprise an epitope, motif or domain associated with CD47.

In one embodiment, the antibodies are bispecific antibodies in which the two chains have different specificities, as described in Millstein et al. Nature. 1983. 305:537-539. Other embodiments include antibodies with additional specificities, such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

As stated above, multivalent antibodies may immunospecifically bind to different epitopes of the desired target molecule or may immunospecifically bind to both the target molecule as well as a heterologous epitope, such as a heterologous polypeptide or solid support material. In some embodiments, the multivalent antibodies may include bispecific antibodies or trispecific antibodies. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art and are disclosed in U.S. Pat. No. 4,676,980, along with a number of cross-linking techniques.

In some embodiments, antibody variable domains with the desired binding specificities (antibody-antigen combining sites) are fused to immunoglobulin constant domain sequences, such as an immunoglobulin heavy chain constant domain comprising at least part of the hinge, CH2, and/or CH3 regions, using methods well known to those of ordinary skill in the art.

### Antibody Derivatives

An antibody or antigen-binding portion thereof provided herein may be further modified to contain additional nonproteinaceous moieties that are known in the art and readily available. The moieties suitable for derivatization of the antibody include but are not limited to water-soluble polymers.

Non-limiting examples of water-soluble polymers include, but are not limited to, PEG, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102:11600-11605 (2005)). The radiation may be of any wavelength and includes, but is not limited to, wavelengths that do not harm ordinary cells, but which heat the nonproteinaceous moiety to a temperature at which cells proximal to the antibody-nonproteinaceous moiety are killed.

Another modification of the antibodies described herein is pegylation. An antibody can be pegylated to, for example, increase the biological (e.g., serum) half-life of the antibody. To pegylate an antibody, the antibody, or fragment thereof, typically is reacted with PEG, such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In some embodiments, the antibody to be pegylated is an aglycosylated antibody. Methods for pegylating proteins are known in the art and can be applied to the antibodies described herein (EP0154 316 by Nishimura et al. and EP0401384 by Ishikawa et al.).

The present disclosure also encompasses a human monoclonal antibody described herein conjugated to a therapeutic agent, a polymer, a detectable label, or enzyme. In one embodiment, the therapeutic agent is a cytotoxic agent. In one embodiment, the polymer is PEG.

### Nucleic Acids, Expression Cassettes, and Vectors

The present disclosure provides isolated nucleic acid segments that encode the polypeptides, peptide fragments, and coupled proteins of the disclosure. The nucleic acid segments of the disclosure also include segments that encode for the same amino acids due to the degeneracy of the genetic code. For example, the amino acid threonine is encoded by ACU, ACC, ACA, and ACG and is therefore degenerate. It is intended that the disclosure includes all variations of the polynucleotide segments that encode for the same amino acids. Such mutations are known in the art (Watson et al., Molecular Biology of the Gene, Benjamin Cummings 1987). Mutations also include alteration of a nucleic acid segment to encode for conservative amino acid changes, for example, the substitution of leucine for isoleucine and so forth. Such mutations are also known in the art. Thus, the genes and nucleotide sequences of the disclosure include both the naturally occurring sequences as well as mutant forms.

The nucleic acid segments of the disclosure may be contained within a vector. A vector may include, but is not limited to, any plasmid, phagemid, F-factor, virus, cosmid, or phage in a double- or single-stranded linear or circular form which may or may not be self transmissible or mobilizable. The vector can also transform a prokaryotic or eukaryotic host either by integration into the cellular genome or exist extra-chromosomally (e.g., autonomous replicating plasmid with an origin of replication).

The nucleic acid segment in the vector can be under the control of, and operably linked to, an appropriate promoter or other regulatory elements for transcription in vitro or in a host cell, such as a eukaryotic cell, or a microbe, e.g., bacteria. The vector may be a shuttle vector that functions in multiple hosts. The vector may also be a cloning vector that typically contains one or a small number of restriction endonuclease recognition sites at which foreign DNA sequences can be inserted in a determinable fashion. Such insertion can occur without loss of essential biological function of the cloning vector. A cloning vector may also contain a marker gene that is suitable for use in the identification and selection of cells transformed with the cloning vector. Examples of marker genes are tetracycline resistance or ampicillin resistance. Many cloning vectors are commercially available (Stratagene, New England Biolabs, Clonetech).

The nucleic acid segments of the present disclosure may also be inserted into an expression vector. Typically, an expression vector contains prokaryotic DNA elements coding for a bacterial replication origin and an antibiotic resistance gene to provide for the amplification and selection of the expression vector in a bacterial host; regulatory elements that control initiation of transcription such as a promoter; and DNA elements that control the processing of transcripts such as introns, or a transcription termination/polyadenylation sequence.

Methods to introduce nucleic acid segment into a vector are available in the art (Sambrook et al., Molecular Cloning: A Laboratory Manual, 3rd edition, Cold Spring Harbor Press, Cold Spring Harbor, N.Y. (2001)). Briefly, a vector into which a nucleic acid segment is to be inserted is treated with one or more restriction enzymes (restriction endonuclease) to produce a linearized vector having a blunt end, a “sticky” end with a 5′ or a 3′ overhang, or any combination of the above. The vector may also be treated with a restriction enzyme and subsequently treated with another modifying enzyme, such as a polymerase, an exonuclease, a phosphatase or a kinase, to create a linearized vector that has characteristics useful for ligation of a nucleic acid segment into the vector. The nucleic acid segment that is to be inserted into the vector is treated with one or more restriction enzymes to create a linearized segment having a blunt end, a “sticky” end with a 5′ or a 3′ overhang, or any combination of the above. The nucleic acid segment may also be treated with a restriction enzyme and subsequently treated with another DNA modifying enzyme. Such DNA modifying enzymes include, but are not limited to, polymerase, exonuclease, phosphatase or a kinase, to create a nucleic acid segment that has characteristics useful for ligation of a nucleic acid segment into the vector.

The treated vector and nucleic acid segment are then ligated together to form a construct containing a nucleic acid segment according to methods available in the art (Sambrook et al., Molecular Cloning: A Laboratory Manual, 3rd edition, Cold Spring Harbor Press, Cold Spring Harbor, N.Y. (2001)). Briefly, the treated nucleic acid fragment, and the treated vector are combined in the presence of a suitable buffer and ligase. The mixture is then incubated under appropriate conditions to allow the ligase to ligate the nucleic acid fragment into the vector.

The disclosure also provides an expression cassette that contains a nucleic acid sequence capable of directing expression of a particular nucleic acid segment of the disclosure, either in vitro or in a host cell. Also, a nucleic acid segment of the disclosure may be inserted into the expression cassette such that an anti-sense message is produced. The expression cassette is an isolatable unit such that the expression cassette may be in linear form and functional for in vitro transcription and translation assays. The materials and procedures to conduct these assays are commercially available from Promega Corp. (Madison, Wis.). For example, an in vitro transcript may be produced by placing a nucleic acid sequence under the control of a T7 promoter and then using T7 RNA polymerase to produce an in vitro transcript. This transcript may then be translated in vitro through use of a rabbit reticulocyte lysate. Alternatively, the expression cassette can be incorporated into a vector allowing for replication and amplification of the expression cassette within a host cell or also in vitro transcription and translation of a nucleic acid segment.

Such an expression cassette may contain one or a plurality of restriction sites allowing for placement of the nucleic acid segment under the regulation of a regulatory sequence. The expression cassette can also contain a termination signal operably linked to the nucleic acid segment as well as regulatory sequences required for proper translation of the nucleic acid segment. The expression cassette containing the nucleic acid segment may be chimeric, meaning that at least one of its components is heterologous with respect to at least one of its other components. The expression cassette may also be one that is naturally occurring but has been obtained in a recombinant form useful for heterologous expression. Expression of the nucleic acid segment in the expression cassette may be under the control of a constitutive promoter or an inducible promoter, which initiates transcription only when the host cell is exposed to some particular external stimulus.

The expression cassette may include in the 5′-3′ direction of transcription, a transcriptional and translational initiation region, a nucleic acid segment and a transcriptional and translational termination region functional in vivo and/or in vitro. The termination region may be native with the transcriptional initiation region, may be native with the nucleic acid segment, or may be derived from another source.

The regulatory sequence can be a polynucleotide sequence located upstream (5′ non-coding sequences), within, or downstream (3′ non-coding sequences) of a coding sequence, and which influences the transcription, RNA processing or stability, or translation of the associated coding sequence. Regulatory sequences can include, but are not limited to, enhancers, promoters, repressor binding sites, translation leader sequences, introns, and polyadenylation signal sequences. They may include natural and synthetic sequences as well as sequences, which may be a combination of synthetic and natural sequences. While regulatory sequences are not limited to promoters, some useful regulatory sequences include constitutive promoters, inducible promoters, regulated promoters, tissue-specific promoters, viral promoters, and synthetic promoters.

A promoter is a nucleotide sequence that controls the expression of the coding sequence by providing the recognition for RNA polymerase and other factors required for proper transcription. A promoter includes a minimal promoter, consisting only of all basal elements needed for transcription initiation, such as a TATA-box and/or initiator that is a short DNA sequence comprised of a TATA-box and other sequences that serve to specify the site of transcription initiation, to which regulatory elements are added for control of expression. A promoter may be derived entirely from a native gene, or be composed of different elements derived from different promoters found in nature, or even be comprised of synthetic DNA segments. A promoter may contain DNA sequences that are involved in the binding of protein factors that control the effectiveness of transcription initiation in response to physiological or developmental conditions.

The disclosure also provides a construct containing a vector and an expression cassette. The vector may be selected from, but not limited to, any vector previously described. Into this vector may be inserted an expression cassette through methods known in the art and previously described (Sambrook et al., Molecular Cloning: A Laboratory Manual, 3rd edition, Cold Spring Harbor Press, Cold Spring Harbor, N.Y. (2001)). In one embodiment, the regulatory sequences of the expression cassette may be derived from a source other than the vector into which the expression cassette is inserted. In another embodiment, a construct containing a vector and an expression cassette is formed upon insertion of a nucleic acid segment of the disclosure into a vector that itself contains regulatory sequences. Thus, an expression cassette is formed upon insertion of the nucleic acid segment into the vector. Vectors containing regulatory sequences are available commercially, and methods for their use are known in the art (Clonetech, Promega, Stratagene).

In another aspect, this disclosure also provides (i) a nucleic acid molecule encoding a polypeptide chain of the antibody or antigen-binding portion thereof described herein; (ii) a vector comprising the nucleic acid molecule as described; and (iii) a cultured host cell comprising the vector as described. Also provided is a method for producing a polypeptide, comprising: (a) obtaining the cultured host cell as described; (b) culturing the cultured host cell in a medium under conditions permitting expression of a polypeptide encoded by the vector and assembling of an antibody or fragment thereof; and (c) purifying the antibody or fragment from the cultured cell or the medium of the cell.

### Methods of Production

Antibodies may be produced using recombinant methods and compositions (U.S. Pat. No. 4,816,567). In one embodiment, an isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, a nucleic acid encoding an antibody, e.g., as described herein, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria (U.S. Pat. Nos. 5,648,237, 5,789,199, and 5,840,523). (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern (Gerngross, Nat. Biotech. 2004. 22:1409-1414 and Li et al., Nat. Biotech. 2006. 24:210-215).

Suitable host cells for the expression of glycosylated antibodies are also derived from multicellular organisms (invertebrates and vertebrates). Examples of invertebrate cells include plant and insect cells. Numerous baculoviral strains have been identified, which may be used in conjunction with insect cells, particularly for transfection of Spodoptera frugiperda cells.

Plant cell cultures can also be utilized as hosts (U.S. Pat. Nos. 5,959,177, 6,040,498, 6,420,548, 7,125,978, and 6,417,429 (describing PLANTIBODIES technology for producing antibodies in transgenic plants)).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include CHO cells, including DHFR-CHO cells (Urlaub et al., Proc. Natl. Acad. Sci. USA 77:4216 (1980)); and myeloma cell lines such as Y0, NS0, and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

### Compositions and Kits

The antibodies or antigen-binding portions thereof described herein represent an excellent way for the development of anti-CD47 antibodies or antigen-binding portions thereof comprising a modified Fc region that activates FcγRs.

In another aspect, the present disclosure provides a pharmaceutical composition comprising the antibodies of the present disclosure described herein formulated together with a pharmaceutically acceptable carrier. The composition may optionally contain one or more additional pharmaceutically active ingredients, such as another antibody or a therapeutic agent.

In some embodiments, the pharmaceutical comprises two or more of the antibody or antigen-binding portion thereof described herein, such as any combinations of the antibody or antigen-binding portion thereof comprising a heavy chain and a light chain that comprise the respective amino acid sequences described herein.

The pharmaceutical compositions of the disclosure also can be administered in a combination therapy with, for example, another immune-stimulatory agent, an antiviral agent, a vaccine, etc. In some embodiments, a composition comprises an antibody or antigen-binding portion thereof of this disclosure at a concentration of at least 1 mg/ml, 5 mg/ml, 10 mg/ml, 50 mg/ml, 100 mg/ml, 150 mg/ml, 200 mg/ml, 1-300 mg/ml, or 100-300 mg/ml.

In some embodiments, the second therapeutic agent comprises, but is not limited to a checkpoint inhibitor or a tumor antigen inhibitor. Examples of checkpoint inhibitors include, but are not limited to those that target PD-1, CTLA-4, PD-L1, and LAG-3. In some embodiments, the checkpoint inhibitor is a drug. In some embodiments, the checkpoint inhibitor is an antibody. In one embodiment, the checkpoint inhibitor is an anti-PD-1 antibody. In some embodiments, the anti-PD-1 antibody includes, but is not limited to nivolumab (OPDIVO®) or pembrolizumab (KEYTRUDA®). Examples of PD-L1 inhibitors include, but are not limited to atezolizumab (TECENTRIQ®), avelumab (BAVENCIO®), and durvalumab (IMFINZI®). Examples of CTLA-4 inhibitors include, but are not limited to ipilimumab (YERVOY®) tremelimumab (IMJUDO®). An example of LAG-3 inhibitors include, but is not limited to relatlimab.

In one embodiment, the tumor antigen is gp75. In some embodiments, the tumor antigen is Melan-A/MART1, PSMA, HER2, HER3, EGFR (mutated or amplified), CD20, CD19, MAGE, BAGE, NY-ESO-1, Nectin-4, TROP-2, Folate Receptor-alpha, Tissue Factor, ROR1, ROR2, B7-H3, CD123, CEACAM5, or NaPi2b.

Also within the scope of this disclosure is use of the pharmaceutical composition in the preparation of a medicament for the treatment of a cancer. In some embodiments, the cancer is selected from the group comprising bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, neoplasm of the central nervous system, lymphoma, leukemia, myeloma, melanoma, sarcoma, and virus-related cancer. In certain embodiments, the cancer is a metastatic cancer, refractory cancer, or recurrent cancer.

The pharmaceutical composition can comprise any number of excipients. Excipients that can be used include carriers, surface-active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

Preferably, a pharmaceutical composition is suitable for intravenous, intramuscular, subcutaneous, parenteral, spinal, intratumoral or epidermal administration (e.g., by injection or infusion). Depending on the route of administration, the active compound can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, intratumoral, transtracheal, subcutaneous, subcuticular, intraarticular, subcapsular, subarachnoid, intraspinal, epidural and intrasternal injection and infusion. Alternatively, an antibody or antigen-binding portion thereof described herein can be administered via a non-parenteral route, such as a topical, epidermal, or mucosal route of administration, e.g., intranasally, orally, vaginally, rectally, sublingually, or topically.

The pharmaceutical compositions of the disclosure may be prepared in many forms that include tablets, hard or soft gelatin capsules, aqueous solutions, suspensions, liposomes, and other slow-release formulations, such as shaped polymeric gels. An oral dosage form may be formulated such that the antibody is released into the intestine after passing through the stomach. Such formulations are described in U.S. Pat. No. 6,306,434 and in the references contained therein.

Oral liquid pharmaceutical compositions may be in the form of, for example, aqueous or oily suspensions, solutions, emulsions, syrups, or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

An antibody can be formulated for parenteral administration (e.g., by injection, for example, bolus injection or continuous infusion) and may be presented in unit dosage form in ampules, prefilled syringes, small volume infusion containers, or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Pharmaceutical compositions suitable for rectal administration can be prepared as unit dose suppositories. Suitable carriers include saline solution and other materials commonly used in the art.

For administration by inhalation, an antibody can be conveniently delivered from an insufflator, nebulizer, a pressurized pack, or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, an antibody may take the form of a dry powder composition, for example, a powder mix of a modulator and a suitable powder base such as lactose or starch. The powder composition may be presented in a unit dosage form in, for example, capsules or cartridges or, e.g., gelatin or blister packs from which the powder may be administered with the aid of an inhalator or insufflator. For intranasal administration, an antibody may be administered via a liquid spray, such as via a plastic bottle atomizer.

Pharmaceutical compositions of the disclosure may also contain other ingredients such as flavorings, colorings, anti-microbial agents, or preservatives. It will be appreciated that the amount of an antibody required for use in treatment will vary not only with the particular carrier selected but also with the route of administration, the nature of the condition being treated, and the age and condition of the patient. Ultimately the attendant health care provider may determine a proper dosage. In addition, a pharmaceutical composition may be formulated as a single unit dosage form.

The pharmaceutical composition of the present disclosure can be in the form of sterile aqueous solutions or dispersions. It can also be formulated in a microemulsion, liposome, or other ordered structure suitable to high drug concentration.

An antibody or antigen-binding portion thereof described herein can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

The amount of active ingredient that can be combined with a carrier material to produce a single dosage form will vary depending upon the subject being treated and the particular mode of administration and will generally be that amount of the composition, which produces a therapeutic effect. Generally, out of one hundred percent, this amount will range from about 0.01% to about 99% of active ingredient, preferably from about 0.1% to about 70%, most preferably from about 1% to about 30% of active ingredient in combination with a pharmaceutically acceptable carrier.

Dosage regimens can be adjusted to provide the optimum desired response (e.g., a therapeutic response). For example, a single bolus can be administered, several divided doses can be administered over time, or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. Alternatively, the antibody can be administered as a sustained release formulation, in which case less frequent administration is required. For administration of the antibody, the dosage ranges from about 0.0001 to 800 mg/kg, and more usually 0.01 to 5 mg/kg, of the host body weight. For example, dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an antibody or antigen-binding portion thereof described herein include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/ml, and in some methods, about 25-300 μg/ml. A “therapeutically effective dosage” of an antibody or antigen-binding portion thereof described herein preferably results in a decrease in severity of disease symptoms, an increase in frequency and duration of disease symptom-free periods, or a prevention of impairment or disability due to the disease affliction. For example, for the treatment of cancer in a subject, a “therapeutically effective dosage” preferably reduces the tumor burden in a subject by at least 30% relative to untreated subjects.

The pharmaceutical composition can be a controlled release formulation, including implants, transdermal patches, and microencapsulated delivery systems. Biodegradable, biocompatible polymers can be used, such as ethylene-vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparati (U.S. Pat. Nos. 4,447,233 and 4,447,224); and (5) osmotic devices (U.S. Pat. Nos. 4,439,196 and 4,475,196); the disclosures of which are incorporated herein by reference.

In some embodiments, the human monoclonal antibodies of the disclosure described herein can be formulated to ensure proper distribution in vivo. For example, to ensure that the therapeutic compounds of the disclosure cross the blood-brain barrier, they can be formulated in liposomes, which may additionally comprise targeting moieties to enhance selective transport to specific cells or organs (U.S. Pat. Nos. 4,522,811; 5,374,548; 5,416,016; and 5,399,331; V.V. Ranade (1989) Clin. Pharmacol. 29:685; Umezawa et al., (1988) Biochem. Biophys. Res. Commun. 153:1038; Bloeman et al. (1995) FEBS Lett. 357:140; M. Owais et al. (1995) Antimicrob. Agents Chemother. 39:180; Briscoe et al. (1995) Am. Physiol. 1233:134; Schreier et al. (1994). Biol. Chem. 269:9090; Keinanen and Laukkanen (1994) FEBS Lett. 346:123; and Killion and Fidler (1994) Immunomethods 4:273).

In some embodiments, the initial dose may be followed by administration of a second or a plurality of subsequent doses of the antibody or antigen-binding portion thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the disclosure, e.g., encapsulation in liposomes, microparticles, microcapsules, recombinant cells capable of expressing the mutant viruses, receptor-mediated endocytosis (Wu et al. J. Biol. Chem. 1987. 262:4429-4432). Methods of introduction include, but are not limited to, intradermal, transdermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can also be delivered in a vesicle, in particular, a liposome (Langer. Science. 1990. 249:1527-1533).

The use of nanoparticles to deliver the antibodies of the present disclosure is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389), incorporated herein by reference. Nanoparticles may be developed and conjugated to antibodies contained in pharmaceutical compositions to target cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous, intracranial, intraperitoneal, intramuscular injections, drip infusions, etc. These injectable preparations may be prepared by methods publicly known. For example, the injectable preparations may be prepared, e.g., by dissolving, suspending, or emulsifying the antibody or its salt described herein in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pens and autoinjector delivery devices have applications in the subcutaneous delivery of a pharmaceutical composition of the present disclosure. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present disclosure include, but certainly are not limited to, the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.) and the HUMIRA™ Pen (Abbott Labs, Abbott Park, IL), to name only a few.

Advantageously, the pharmaceutical compositions for oral or parenteral use described herein are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg per dosage form in a unit dose; especially in the form of injection, it is preferred that the antibody is contained in about 5 to about 300 mg and in about 10 to about 300 mg for the other dosage forms.

In another aspect, this disclosure provides a kit comprising a pharmaceutically acceptable dose unit of the antibody or antigen-binding portion thereof or the pharmaceutical composition as described herein.

In some embodiments, the kit also includes a container that contains the composition and optionally informational material. The informational material can be descriptive, instructional, marketing, or other material that relates to the methods described herein and/or the use of the agents for therapeutic benefit. In an embodiment, the kit also includes an additional therapeutic agent, as described herein. For example, the kit includes a first container that contains the composition and a second container for the additional therapeutic agent.

The informational material of the kits is not limited in its form. In some embodiments, the informational material can include information about production of the composition, concentration, date of expiration, batch or production site information, and so forth. In one embodiment, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, audio recording, or information that contains a link or address to substantive material.

The kit can include one or more containers for the composition. In some embodiments, the kit contains separate containers, dividers, or compartments for the composition and informational material. For example, the composition can be contained in a bottle or vial, and the informational material can be contained in a plastic sleeve or packet. In other embodiments, the separate elements of the kit are contained within a single, undivided container. For example, the composition is contained in a bottle or vial that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition or other suitable delivery device. The device can be provided pre-loaded with one or both of the agents or can be empty, but suitable for loading. Such a kit may optionally contain a syringe to allow for injection of the antibody contained within the kit into an animal, such as a human.

### Methods of Use

### Methods of Treatment

In some embodiments, an anti-CD47 antibody or antigen-binding portion described herein is used in a method of treating cancer in a subject in need thereof. In some embodiments, the cancer is selected from the group comprising bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, neoplasm of the central nervous system, lymphoma, leukemia, myeloma, melanoma, sarcoma, and virus-related cancer. In certain embodiments, the cancer is a metastatic cancer, refractory cancer, or recurrent cancer.

In some embodiments, the anti-CD47 antibody or antigen-binding portion described herein is used in a method of decreasing tumor burden in a subject in need thereof. In some embodiments, the tumor is malignant.

In some embodiments, the anti-CD47 antibody or antigen-binding portion described herein is used in a method of boosting systemic antitumor immunity with minimal toxicity in a subject in need thereof.

In some embodiments, the anti-CD47 antibody or antigen-binding portion described herein is used in a method of blocking human CD47 to alter the tumor microenvironment effector cell composition.

### Combination Therapies

In some embodiments, the anti-CD47 antibody or antigen-binding portion described herein is used in combination with another therapeutic agent. In some embodiments, the therapeutic agent is a tumor antigen inhibitor or a checkpoint inhibitor. Examples of checkpoint inhibitors include, but are not limited to those that target PD-1, CTLA-4, PD-L1, and LAG-3. In some embodiments, the checkpoint inhibitor is a drug. In some embodiments, the checkpoint inhibitor is an antibody. In one embodiment, the checkpoint inhibitor is an anti-PD-1 antibody. In some embodiments, the anti-PD-1 antibody includes, but is not limited to nivolumab (OPDIVO®) or pembrolizumab (KEYTRUDA®). Examples of PD-L1 inhibitors include, but are not limited to atezolizumab (TECENTRIQ®), avelumab (BAVENCIO®), and durvalumab (IMFINZI®). Examples of CTLA-4 inhibitors include, but are not limited to ipilimumab (YERVOY®) tremelimumab (IMJUDO®). An example of LAG-3 inhibitors include, but is not limited to relatlimab.

In some embodiments, the therapeutic agent is a chemotherapeutic agent. Examples of chemotherapy include, but are not limited to camptothecin (CPT-11), 5-fluorouracil (5-FU), cisplatin, doxorubicin, irinotecan, paclitaxel, gemcitabine, cisplatin, paclitaxel, carboplatin-paclitaxel (Taxol), doxorubicin, 5-fu, and camptothecin+apo21/TRAIL (a 6× combo).

In one embodiment, the tumor antigen is gp75. In some embodiments, the tumor antigen is Melan-A/MART1, PSMA, HER2, HER3, EGFR (mutated or amplified), CD20, CD19, MAGE, BAGE, NY-ESO-1, Nectin-4, TROP-2, Folate Receptor-alpha, Tissue Factor, ROR1, ROR2, B7-H3, CD123, CEACAM5, or NaPi2b.

As used herein, the term “in combination with” means that additional therapeutically active component(s) may be administered prior to, concurrent with, or after the administration of the anti-CD47 antibody or antigen-binding portion thereof of the present disclosure. The term “in combination with” also includes sequential or concomitant administration of an anti-CD47 antibody or antigen-binding portion thereof and a second therapeutic agent.

The additional therapeutic agent may be administered to a subject prior to administration of an anti-CD47 antibody or antigen-binding portion thereof of the present disclosure. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of an anti-CD47 antibody or antigen-binding portion thereof of the present disclosure. For example, a first therapeutic agent may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component. In yet other embodiments, the additional therapeutic agent may be administered to a subject concurrent with administration of an anti-CD47 antibody or antigen-binding portion thereof described herein. “Concurrent” administration, for purposes of the present disclosure, includes, e.g., administration of an anti-CD47 antibody or antigen-binding portion thereof antibody and an additional therapeutic agent to a subject in a single dosage form, or in separate dosage forms administered to the subject within about 30 minutes or less of each other. If administered in separate dosage forms, each dosage form may be administered via the same route (e.g., both the anti-CD47 antibody or antigen-binding portion thereof and the additional therapeutic agent may be administered intravenously, etc.); alternatively, each dosage form may be administered via a different route (e.g., the anti-CD47 antibody or antigen-binding portion thereof may be administered intravenously, and the additional therapeutic agent may be administered orally). In any event, administering the components in a single dosage form, in separate dosage forms by the same route, or in separate dosage forms by different routes are all considered “concurrent administration,” for purposes of the present disclosure. For purposes of the present disclosure, administration of an anti-CD47 antibody or antigen-binding portion thereof “prior to,” “concurrent with,” or “after” (as those terms are defined hereinabove) administration of an additional therapeutically active component is considered administration of an anti-CD47 antibody or antigen-binding portion thereof “in combination with” an additional therapeutic agent.

The present disclosure includes pharmaceutical compositions in which an anti-CD47 antibody or antigen-binding portion thereof described herein is co-formulated with one or more of the additional therapeutic agent(s) as described elsewhere herein.

### Administration Regimens

According to certain embodiments, a single dose of an anti-CD47 antibody or antigen-binding portion thereof described herein (or a pharmaceutical composition comprising a combination of an anti-CD47 antibody or antigen-binding portion thereof and any of the additional therapeutic agents mentioned herein) may be administered to a subject in need thereof. According to certain embodiments of the present disclosure, multiple doses of an anti-anti-CD47 antibody or antigen-binding portion thereof (or a pharmaceutical composition comprising a combination of an anti-CD47 antibody or antigen-binding portion thereof and any of the additional therapeutic agents described herein) may be administered to a subject over a defined time course. The methods according to this aspect of the disclosure comprise sequentially administering to a subject multiple doses of an anti-CD47 antibody or antigen-binding portion thereof described herein. As used herein, “sequentially administering” means that each dose of an anti-CD47 antibody or antigen-binding portion thereof is administered to the subject at a different point in time, e.g., on different days separated by a predetermined interval (e.g., hours, days, weeks, or months). The present disclosure includes methods that comprise sequentially administering to the patient a single initial dose of an anti-CD47 antibody or antigen-binding portion thereof, followed by one or more secondary doses of the anti-CD47 antibody or antigen-binding portion thereof, and optionally followed by one or more tertiary doses of the anti-CD47 antibody or antigen-binding portion thereof.

The terms “initial dose,” “secondary doses,” and “tertiary doses,” refer to the temporal sequence of administration of the anti-CD47 antibody or antigen-binding portion thereof described herein. Thus, the “initial dose” is the dose, which is administered at the beginning of the treatment regimen (also referred to as the “baseline dose”); the “secondary doses” are the doses, which are administered after the initial dose; and the “tertiary doses” are the doses which are administered after the secondary doses. The initial, secondary, and tertiary doses may all contain the same amount of anti-CD47 antibody or antigen-binding portion thereof, but generally may differ from one another in terms of frequency of administration. In some embodiments, however, the amount of anti-CD47 antibody or antigen-binding portion thereof contained in the initial, secondary and/or tertiary doses varies from one another (e.g., adjusted up or down as appropriate) during the course of treatment. In some embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present disclosure, each secondary and/or tertiary dose is administered 1 to 48 hours (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) after the immediately preceding dose. The phrase “the immediately preceding dose,” as used herein, means, in a sequence of multiple administrations, the dose of anti-CD47 antibody or antigen-binding portion thereof, which is administered to a patient prior to the administration of the very next dose in the sequence with no intervening doses.

The methods, according to this aspect of the disclosure, may comprise administering to a patient any number of secondary and/or tertiary doses of an anti-CD47 antibody or antigen-binding portion thereof. For example, in some embodiments, only a single secondary dose is administered to the patient. In other embodiments, two or more (e.g., 2, 3, 4, 5, 6, 7, 8, or more) secondary doses are administered to the patient. Likewise, in some embodiments, only a single tertiary dose is administered to the patient. In other embodiments, two or more (e.g., 2, 3, 4, 5, 6, 7, 8, or more) tertiary doses are administered to the patient.

In some embodiments, the frequency at which the secondary and/or tertiary doses are administered to a patient can vary over the course of the treatment regimen. The frequency of administration may also be adjusted during the course of treatment by a physician depending on the needs of the individual patient following clinical examination.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “antibody” as referred to herein includes whole antibodies and any antigen-binding portion or single chains thereof. Whole antibodies are glycoproteins comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFRl, HCDRl, HFR2, HCDR2, HFR3, HCDR3, HFR4. The light chain variable region CDRs and FRs are LFRl, LCDRl, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

The term “antigen-binding portion” or “antigen binding fragment” of an antibody, as used herein, refers to one or more fragments of an antibody that retain the ability to specifically bind to an antigen (e.g., human CD47). Examples of binding fragments encompassed within the term “antigen-binding portion/fragment” of an antibody include (i) a Fab fragment—a monovalent fragment consisting of the VL, VH, CL and CHI domains; (ii) a F(ab′)2 fragment—a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, and (v) a dAb fragment (Ward et al. Nature. 1989. 341:544-546) consisting of a VH domain. An isolated complementarity determining region (CDR), or a combination of two or more isolated CDRs joined by a synthetic linker, may comprise and antigen binding domain of an antibody if able to bind antigen.

Unless otherwise indicated, the word “fragment” when used with reference to an antibody, such as in a claim, refers to an antigen binding fragment of the antibody, such that “antibody or fragment” has the same meaning as “antibody or antigen binding fragment thereof.”

An “isolated antibody,” as used herein, is intended to refer to an antibody that is substantially free of other antibodies having different antigenic specificities (e.g., an isolated antibody that specifically binds to human CD47). An isolated antibody can be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody” is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies or antigen-binding fragments thereof described herein can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity, which have variable regions in which both the framework and CDR regions are derived from human germline immunoglobulin sequences. In one embodiment, the human monoclonal antibodies can be produced by a hybridoma that includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created, or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In some embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “isotype” refers to the antibody class (e.g., IgM or IgG1) that is encoded by the heavy chain constant region genes. The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody. The term “humanized antibody” is intended to refer to antibodies in which CDR sequences derived from the germline of another mammalian species, such as a mouse, have been grafted onto human framework sequences. Additional framework region modifications can be made within the human framework sequences.

The term “chimeric antibody” is intended to refer to antibodies in which the variable region sequences are derived from one species, and the constant region sequences are derived from another species, such as an antibody in which the variable region sequences are derived from a mouse antibody, and the constant region sequences are derived from a human antibody. The term can also refer to an antibody in which its variable region sequence or CDR(s) is derived from one source (e.g., an IgA1 antibody), and the constant region sequence or Fc is derived from a different source (e.g., a different antibody, such as an IgG, IgA2, IgD, IgE or IgM antibody).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIb, or equivalently FcγRIIB) receptor. Various properties of human FcγRs are summarized in Table 2. The majority of innate effector cell types co-express one or more activating FcγR and the inhibitory FcγRIIb, whereas natural killer (NK) cells selectively express one activating Fc receptor (FcγRIII in mice and FcγRIIIA in humans) but not the inhibitory FcγRIIb in mice and humans. Human IgG1 binds to most human Fc receptors and is considered equivalent to murine IgG2a with respect to the types of activating Fc receptors that it binds to.

TABLE 2
Properties of Human FcγRs

| | Allelic | Affinity for | Isotype | Cellular |
|---|---|---|---|---|
| Fcγ | variants | human IgG | preference | distribution |
| FcγRI | None | High (K_{D} ~10 | IgG1 = 3 > 4 >> 2 | Monocytes, macrophages, |
| | described | nM) | | activated neutrophils, |
| | | | | dendritic cells? |
| FcγRIIA | H131 | Low to medium | IgG1 > 3 > 2 > 4 | Neutrophils, monocytes, |
| | R131 | Low | IgG1 > 3 > 4 > 2 | macrophages, eosinophils, |
| | | | | dendritic cells, platelets |
| FcγRIIIA | V158 | Medium | IgG1 = 3 >> 4 > 2 | NK cells, monocytes, |
| | F158 | Low | IgG1 = 3 >> 4 > 2 | macrophages, mast cells, |
| | | | | eosinophils, dendritic cells? |
| FcγRIIb | I232 | Low | IgG1 = 3 = 4 > 2 | B cells, monocytes, |
| | T232 | Low | IgG1 = 3 = 4 > 2 | macrophages, dendritic cells, |
| | | | | mast cells |

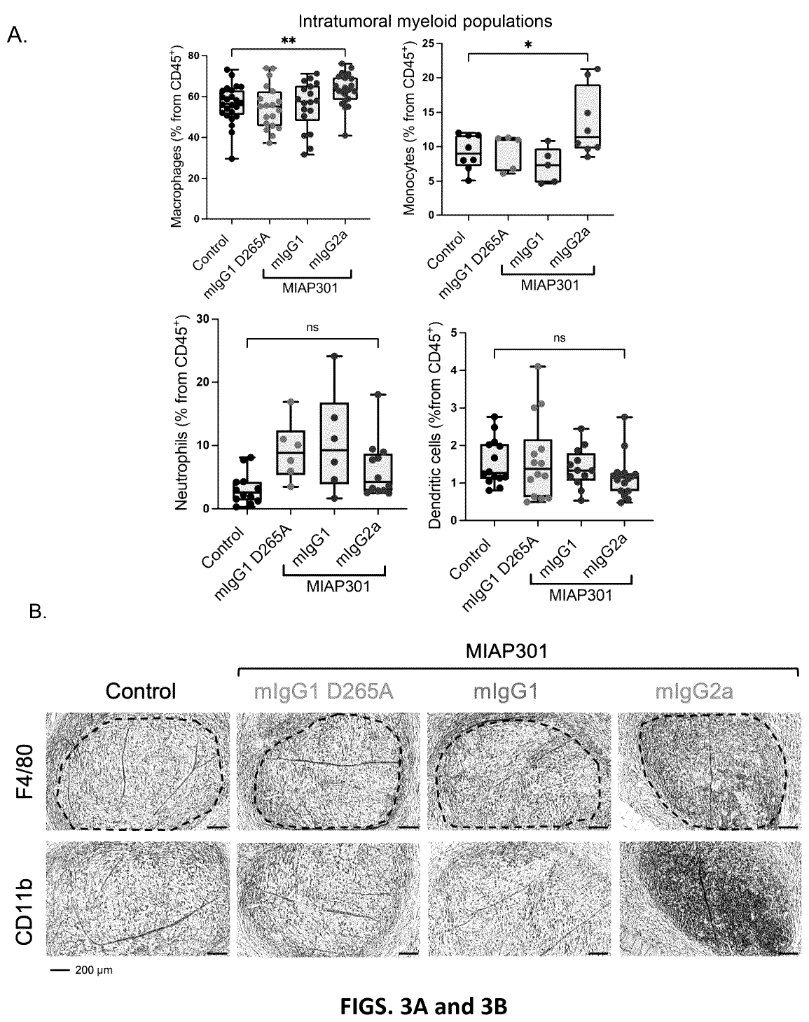

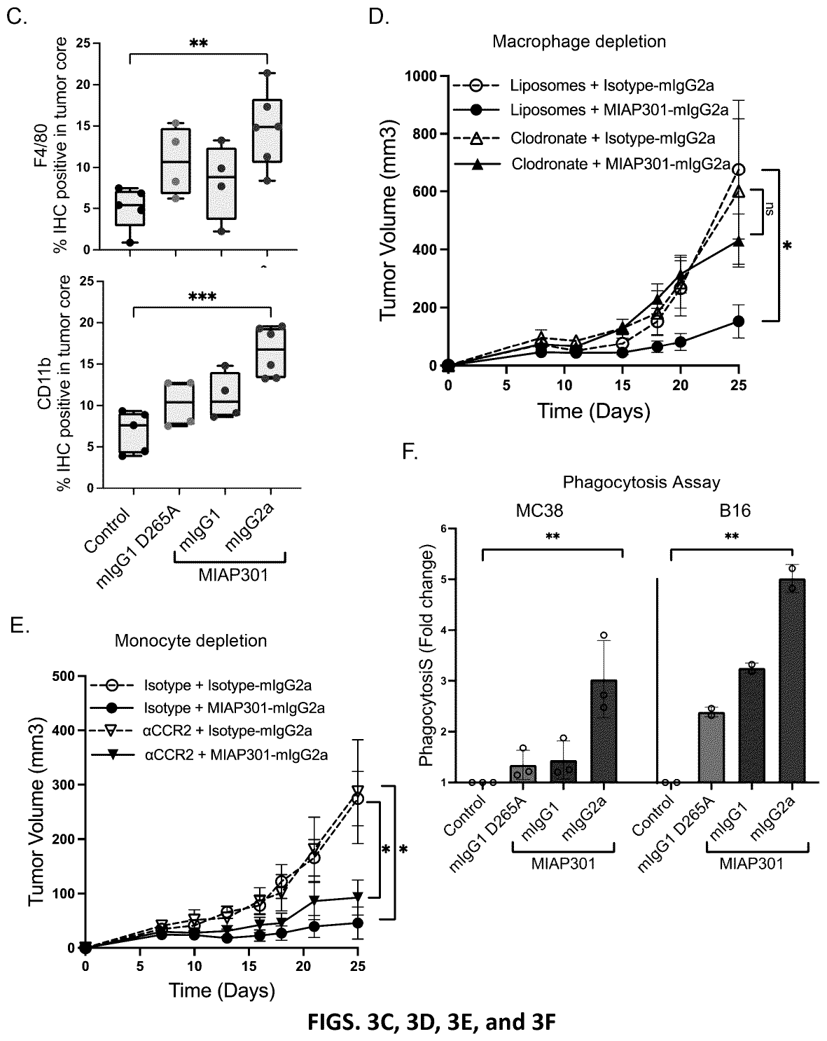

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (Clq) of the classical complement system. Thus, an Fc region comprises the constant region of an antibody excluding the first constant region immunoglobulin domain (e.g., CH1 or CL). In IgG, IgA and IgD antibody isotypes, the Fc region comprises CH2 and CH3 constant domains in each of the antibody's two heavy chains; IgM and IgE Fc regions comprise three heavy chain constant domains (CH domains 2-4) in each polypeptide chain. For IgG, the Fc region comprises immunoglobulin domains Cy2 and Cy3 and the hinge between Cyl and Cy2. Although the boundaries of the Fc region of an immunoglobulin heavy chain might vary, the human IgG heavy chain Fc region is usually defined to stretch from an amino acid residue at position C226 or P230 (or an amino acid between these two amino acids) to the carboxy-terminus of the heavy chain, wherein the numbering is according to the EU index as in Kabat. Kabat et al. (1991) Sequences of Proteins of Immunological Interest, National Institutes of Health, Bethesda, Md.; see also FIGS. 3c-3f of U.S. Pat. App. Pub. No. 2008/0248028. The CH2 domain of a human IgG Fc region extends from about amino acid 231 to about amino acid 340, whereas the CH3 domain is positioned on C-terminal side of a CH2 domain in an Fc region, i.e., it extends from about amino acid 341 to about amino acid 447 of an IgG (including a C-terminal lysine). As used herein, the Fc region may be a native sequence Fc, including any allotypic variant, or a variant Fc (e.g., a non-naturally occurring Fc). Fc may also refer to this region in isolation or in the context of an Fc-comprising protein polypeptide such as a “binding protein comprising an Fc region,” also referred to as an “Fc fusion protein” (e.g., an antibody or immunoadhesin).

The disclosure encompasses isolated or substantially purified nucleic acids, peptides, polypeptides, or proteins. In the context of the present disclosure, an “isolated” nucleic acid, DNA or RNA molecule or an “isolated” polypeptide is a nucleic acid, DNA molecule, RNA molecule, or polypeptide that exists apart from its native environment and is therefore not a product of nature. An isolated nucleic acid, DNA molecule, RNA molecule, or polypeptide may exist in a purified form or may exist in a non-native environment such as, for example, a transgenic host cell. A “purified” nucleic acid molecule, peptide, polypeptide, or protein, or a fragment thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein, peptide, or polypeptide that is substantially free of cellular material includes preparations of protein, peptide, or polypeptide having less than about 30%, 20%, 10%, or 5% (by dry weight) of contaminating protein. When the protein of the disclosure, or biologically active portion thereof, is recombinantly produced, preferably culture medium represents less than about 30%, 20%, 10%, or 5% (by dry weight) of chemical precursors or non-protein-of-interest chemicals.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A peptide or polypeptide “fragment” as used herein refers to a less than full-length peptide, polypeptide, or protein. For example, a peptide or polypeptide fragment can have at least about 3, at least about 4, at least about 5, at least about 10, at least about 20, at least about 30, at least about 40 amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids, or less than about 250 amino acids in length. Preferably the peptide fragment can elicit an immune response when used to inoculate an animal. A peptide fragment may be used to elicit an immune response by inoculating an animal with a peptide fragment in combination with an adjuvant, a peptide fragment that is coupled to an adjuvant, or a peptide fragment that is coupled to arsanilic acid, sulfanilic acid, an acetyl group, or a picryl group. A peptide fragment can include a non-amide bond and can be a peptidomimetic.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The term “recombinant,” as used herein, refers to antibodies or antigen-binding portions thereof of the disclosure created, expressed, isolated, or obtained by technologies or methods known in the art as recombinant DNA technology, which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art (Pearson (1994) Methods Mol. Biol. 24:307-331, which is herein incorporated by reference). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. Science. 1992. 256:1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters (Altschul et al. J. Mol. Biol. 1990. 215:403-410 and (1997) Nucleic Acids Res. 25:3389-3402, each of which is herein incorporated by reference).

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein.

The term “specifically binds,” “binds specifically to,” or the like, refers to an antibody that binds to a single epitope, e.g., under physiologic conditions., but which does not bind to more than one epitope. Accordingly, an antibody that specifically binds to a polypeptide will bind to an epitope that is present on the polypeptide, but which is not present on other polypeptides. Specific binding can be characterized by an equilibrium dissociation constant of at least about 1×10⁻⁸ M or less (e.g., a smaller KD denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™ which bind specifically to human CD47.

Accordingly, an antibody that “specifically binds to human CD47” refers to an antibody that binds to soluble or cell bound human CD47 with a KD of 10⁻⁻⁷ M or less, such as approximately less than 10⁻⁻⁸ M, 10⁻⁻⁹ M or 10⁻⁻¹⁰ M or even lower.

The term “epitope” as used herein refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In some embodiments, epitopes may include determinants that are chemically active surface groupings of molecules such as amino acids, sugar side chains, phosphoryl groups, or sulfonyl groups, and, in some embodiments, may have specific three-dimensional structural characteristics, and/or specific charge characteristics. An epitope typically includes at least 3, 4, 5, 6, 7, 8, 9, 10, 11, 12, 13, 14, or 15 amino acids in a unique spatial conformation. Methods for determining what epitopes are bound by a given antibody (i.e., epitope mapping) are well known in the art and include, for example, immunoblotting and immune-precipitation assays, wherein overlapping or contiguous peptides from a spike or S protein are tested for reactivity with a given antibody. Methods of determining spatial conformation of epitopes include techniques in the art and those described herein, for example, x-ray crystallography and 2-dimensional nuclear magnetic resonance (Epitope Mapping Protocols in Methods in Molecular Biology, Vol. 66, G. E. Morris, Ed. (1996)).

The term “epitope mapping” refers to the process of identification of the molecular determinants for antibody-antigen recognition.

The term “binds to an epitope” or “recognizes an epitope” with reference to an antibody or antibody fragment refers to continuous or discontinuous segments of amino acids within an antigen. Those of skill in the art understand that the terms do not necessarily mean that the antibody or antibody fragment is in direct contact with every amino acid within an epitope sequence.

The term “binds to the same epitope” with reference to two or more antibodies means that the antibodies bind to the same, overlapping, or encompassing continuous or discontinuous segments of amino acids. Those of skill in the art understand that the phrase “binds to the same epitope” does not necessarily mean that the antibodies bind to or contact exactly the same amino acids. The precise amino acids that the antibodies contact can differ.

For example, a first antibody can bind to a segment of amino acids that is completely encompassed by the segment of amino acids bound by a second antibody. In another example, a first antibody binds one or more segments of amino acids that significantly overlap the one or more segments bound by the second antibody. For the purposes herein, such antibodies are considered to “bind to the same epitope.”

As used herein, the term “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell, or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell.

In many embodiments, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human. As used herein, the expression “a subject in need thereof” or “a patient in need thereof” means a human or non-human mammal that exhibits one or more symptoms or indications of disorders (e.g., cancer), and/or who has been diagnosed with inflammatory disorders. In some embodiments, the subject is a mammal. In some embodiments, the subject is human.

As used herein, “cancer” refers a broad group of diseases characterized by the uncontrolled growth of abnormal cells in the body. Unregulated cell division may result in the formation of malignant tumors or cells that invade neighboring tissues and may metastasize to distant parts of the body through the lymphatic system or bloodstream.

As used herein, the term “disease” is intended to be generally synonymous and is used interchangeably with the terms “disorder” and “condition” (as in medical condition), in that all reflect an abnormal condition (e.g., inflammatory disorder) of the human or animal body or of one of its parts that impairs normal functioning, is typically manifested by distinguishing signs and symptoms, and causes the human or animal to have a reduced duration or quality of life.

As used herein, the term “treating” or “treatment” of any disease or disorder refers in one embodiment, to ameliorating the disease or disorder (i.e., arresting or reducing the development of the disease or at least one of the clinical symptoms thereof). In another embodiment, “treating” or “treatment” refers to ameliorating at least one physical parameter, which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

The terms “prevent,” “preventing,” “prevention,” “prophylactic treatment” and the like refer to reducing the probability of developing a disorder or condition in a subject, who does not have, but is at risk of or susceptible to developing a disorder or condition.

The terms “decrease,” “reduced,” “reduction,” “decrease,” or “inhibit” are all used herein generally to mean a decrease by a statistically significant amount. However, for avoidance of doubt, “reduced,” “reduction,” “decrease,” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “agent” denotes a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

The term “therapeutic effect” is art-recognized and refers to a local or systemic effect in animals, particularly mammals, and more particularly humans caused by a pharmacologically active substance.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

As used herein, the term “composition” or “pharmaceutical composition” refers to a mixture of at least one component useful within the disclosure with other components, such as carriers, stabilizers, diluents, dispersing agents, suspending agents, thickening agents, and/or excipients. The pharmaceutical composition facilitates administration of one or more components of the disclosure to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of this disclosure, and are physiologically acceptable to the subject. Supplementary active compounds may also be incorporated into the compositions.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time (Kohrt et al. Blood. 2011. 117:2423).

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest, such as antibodies. The sample may be any tissue sample from the subject. The sample may comprise protein from the subject.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition that is “substantially free” from Y may be completely free from Y. Where necessary, the word “substantially” may be omitted from the definition of the disclosure.

As used herein, the term “each,” when used in reference to a collection of items, is intended to identify an individual item in the collection, but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1. Materials and Methods

This Example details the materials and methods used in Examples 2-8.

### Example 1A. Mouse Strains

C57BL/6J (WT) (Strain #: 000664) mice were purchased from The Jackson Laboratory. Mice humanized for FcγRs (hFcγRs mouse: murine a chain KO, FcγRI+, FcγRIIAR131+, FcγRIIB+, FcγRIIIAF158+, FcγRIIIB+) were generated and extensively characterized in our laboratory as previously described (Smith, P. et al. PNAS. 2012. 109, 6181-6186). The Fc receptor common γ-chain deficient mice (FcR-γ chain-) were generated as previously described (Takai, T. et al. Cell. 1994. 76, 519-529).

Mice humanized for CD47 and SIRPα (hCD47/hSIRPα KI) were generated independently on the C57BL/6J background by CRISPR/Cas9-mediated gene-targeting strategy. For each gene, the extracellular domain of mouse CD47 (exon 2) and mouse SIRPα (exon 2) were excised by sgRNAs and replaced with their respective human exon 2 transgene sequences (via synthesized MegaMers, IDT) through non-homologous end joining (NHEJ). To target the mouse CD47 and SIRPα genes, CRISPR sgRNAs (UpCD47C-TTGCATCGTCCGTAATGTGGAGG (SEQ ID NO: 1), DnCD47D-ATAGAGCTGAAAAACCGCACGGG (SEQ ID NO: 2) and UpSIRPaA-AAATCAGTGTCTGTTGCTGCTGG (SEQ ID NO: 3), UpSIRPaE-GGAACAGAGGTCTATGTACTCGG (SEQ ID NO: 4) (PAMS in bold) were used to guide the Alt-R™ S.p. HiFi Cas9 Nuclease (IDT) creating double stranded breaks.

To create the humanized CD47 KI, the corresponding CRISPR/Cas9 reagents were microinjected into C57BL/6 (Jackson Laboratories) 1-cell stage mouse embryos and then implanted into surrogate CD-1 mice (Charles River Laboratories). Pups born were screened for presence of the targeted allele and analyzed for proper expression. The transgenesis of the humanized SIRPα KI utilized a different approach to deliver the CRISPR/Cas9 kit called “improved-Genome editing via Oviduct Nucleic Acids Delivery (i-Gonad) (Ohtsuka, M. et al. Genome Biol. 2018. 19, 25. 10.1186/s13059-018-1400-x). Similarly, pups born from recipient female mice were screened for the presence of the Exon 2 KI and analyzed for proper expression. Mice humanized for hCD47 and hSIRPα (hCD47/hSIRPα KI) were generated by backcrossing a hCD47 KI mouse to a hSIRPα KI mouse. Mice humanized for CD47, SIRPα and FcγRs (hCD47/hSIRPα/hFcγR) were generated by backcrossing a fully hCD47/hSIRPα KI mouse to hFcγR mice. All mice were bred and housed at Rockefeller University Comparative Bioscience Center under specific pathogen-free conditions. All mice used were 7 to 12-week-old at the time of the experiment, with a mix of male and female mice. All experiments were performed in compliance with institutional guidelines and were approved by the Rockefeller University Institutional Animal Care and Use Committee (IACUC).

### Example 1B. Cell Lines and Cell Culture

Murine MC38 colorectal cancer, B16 melanoma, Lewis Lung carcinoma (LLC) and human Jurkat and Raji lymphoma cells and were obtained from ATCC. HKP1 (KrasG12Dp53^{-/-}) lung adenocarcinoma cells were characterized and obtained from the laboratory of Dr. Vivek Mittal (Markowitz, G. J. et al. JCI Insight. 2018. 3. 10.1172/jci.insight.96836). All murine cells were maintained in DMEM (Life Technologies) supplemented with 10% fetal bovine serum (Life Technologies), 100 U/mL of penicillin, and 100 μg/mL of streptomycin (Life Technologies). Jurkat and Raji cells were maintained in RPMI-1640 Medium supplemented with 10% fetal bovine serum (Life Technologies, and 100 U/mL of penicillin, and 100 μg/mL of streptomycin (Life Technologies). Cells were split twice per week and cell viability was measured using trypan blue staining in the Countess II automated cell counter (Thermo Fisher). MC38 hCD47 KI and B16 hCD47 KI cancer cells were generated through CRISPR/Cas9-mediated gene targeting by replacing the extracellular domain of mouse CD47 (exon 2) by their respective human counterpart through non-homologous end joining. CRISPR sgRNA (U*C*U*GCAUAUAUGAUUAUCUU (SEQ ID NO: 5) and A*C*C*UUGCAGAAGUCACUAG (SEQ ID NO: 6)) were synthesized and purchased from Synthego. hCD47 (exon 2) HRD was purchased from Integrated DNA technologies (IDT). Cells were subsequently sorted using anti-hCD47 and anti-mCD47 antibodies, to obtain cells that homogeneously expressed only hCD47.

### Example 1C. Antibody Engineering and Expression

The variable heavy and light regions of anti-hCD47 ab clone 5F9 were synthesized (IDT) based on its published sequence (See SEQ ID NOs: 18 and 19 of U.S. Pat. No. 9,382,320). The variable heavy and light regions of the anti-CD47 antibodies MIAP301 (anti-mCD47 ab) and 2D3 (nonblocking anti-hCD47 ab) were decoded by mass spectrometry analysis (Bioinformatics Solutions, Inc.). The variable region sequences of the parental antibodies were subcloned and inserted into mammalian expression vectors with hIgG1, hIgG4, mIgG2a, mIgG1 heavy chains, or human k or mouse k light chains, as previously described (Li, F. et al. Science. 2011. 333, 1030-1034).

For the generation of Fc-domain variants of hIgG1 (GAALIE: G236A/A330L/1332E) and mIgG1 (D265A), site-directed mutagenesis using specific primers was performed based on the QuikChange site-directed mutagenesis Kit II (Agilent Technologies) according to the manufacturer's instructions. Mutated plasmid sequences were validated by direct sequencing (Genewiz).

Antibodies were generated by transient cotransfection of Expi293F cells with heavy-chain and light-chain constructs. Expi293F cells were maintained in serum-free Expi293 Expression Medium and transfected using an ExpiFectamine 293 Transfection Kit (all from Thermo Fischer Scientific). Supernatants were collected 7 days after transection, centrifuged, and filtered (0.22 μm). Antibodies were purified from clarified supernatants using Protein G Sepharose 4 Fast Flow (GE Healthcare), dialyzed in PBS, and sterile filtered (0.22 μm) as previously described (Nimmerjahn, F. et al. Immunity. 2005. 23, 41-51).

### Example 1D. ELISA Assays

Binding specificity of antibodies targeting mouse (MIAP301) and human (2D3 and 5F9) monoclonal antibodies were determined by ELISA using recombinant mouse (SinoBiological 57231-MNAH) and human (SinoBiological 12283-HCCH) CD47, respectively. Blocking activity of 5F9-mIgG2a and 2D3-mIgG2a was determined by competitive ELISA using recombinant human SIRPα-hFc (SinoBiological 11612-H02H1). The 96 well ELISA Half Area High Binding plates (Greiner Bio-One, #675061) were coated overnight at 4° C. with recombinant CD47 proteins (1 μg/mL). All sequential steps were performed at room temperature. After washing, the plates were blocked for 1 hour with 1×PBS containing 2% BSA) and were subsequently incubated for 1 hour with serially diluted IgGs (dilutions are indicated in the figures and were prepared in blocking solution). After washing, plates were incubated for 1 hour with horseradish peroxidase conjugated anti-human or anti-mouse IgG (Jackson IummunoResearch). For competitive ELISAs, after blocking nonspecific sites, plates were incubated for 1 hour with 1 μg/mL of human SIRPα-hFc in 1×PBS with 1% BSA. After washing, plates were incubated for 1 hour with serially diluted 5F9-mIgG2a or 2D3-mIgG2a in 1×PBS with 1% BSA. After washing, plates were incubated for 1 hour with horseradish peroxidase conjugated anti-human IgG (#109-036-088, Jackson IummunoResearch). Detection was performed using a one component substrate solution (TMB), and reactions stopped with the addition of 2 M phosphoric acid. Absorbance at 450 nm was immediately recorded using a SpectraMax Plus spectrophotometer (Molecular Devices), and background absorbance from negative control samples was subtracted.

### Example 1E. Tumor Challenge, Antibody Treatments, and Myeloid Cell Depletion

MC38 (2×10⁶ cell/mouse), MC38-hCD47KI (5×10⁶ cell/mouse), B16 (5×10⁵ cell/mouse), or B16-hCD47KI (5×10⁵ cell/mouse) were inoculated subcutaneously and tumor volumes were measured biweekly with an electronic caliper and reported as volume (mm3) using the formula (L12×L2)/2, where L1 is the shortest diameter and L2 is the longest diameter. Mice were randomized and treated with systemic (intraperitoneal), or IT injections as indicated in the figures. A total of 5-10 biological replicates per group were done in each experiment. For the lung metastases model, B16 or B16-hCD47KI (5×10⁵ cell/mouse) were inoculated IV into the lateral tail vein in 200 μL PBS. Mice were randomized and received intraperitoneal (IP) injections of 20 mg/Kg on days 1, 4, 7, and 11 after inoculation. The lungs were harvested on day 14 and analyzed for the presence of surface metastatic foci using a dissecting microscope.

Macrophage depletion was performed by IT administration of clodronate liposomes (100 μL/mouse; Catalog F70101C-2, Formumax Scientific Inc) or control liposomes on days 4 and 7 after tumor cell inoculation. CCR2+ monocyte depletion was performed by systemic administration of anti-CCR2 ab (Clone MC-21, 100 μg) or isotype control on day 7 after tumor inoculation. CD8+ T cell depletion was performed by systemic administration of anti-CD8 ab (Clone 2.43, 100 μg, BioXcell) or isotype control on day 7 after tumor inoculation.

### Example 1F. Tissue Processing

Tumors were dissected and cut into small pieces and transferred to gentleMACS C tubes (Miltenyi Biotec) containing enzyme mix for tough tumors (Catalog 130-096-730m Miltenyi Biotec) in Dulbecco's modified Eagle's medium (DMEM) (Biological Industries). Tumors were then dissociated using the gentleMACS OctoDissociator with Heaters (gentleMACS Program 37Cm_TDK_2, Miltenyi Biotec). Cell suspensions were then dispersed through a 70-μm nylon cell strainer and washed with PBS.

### Example 1G. Flow Cytometry

Surface expression of mouse or human CD47 on murine tumor cell lines was assessed using MIAP301-PE (Biolegend) and CC2C6-APC (Biolegend) or 5F9-APC (generated in-house) antibodies, respectively. Tumor cells (5× 10⁵) were incubated with 0.5 g of ab cocktail. Baseline staining was obtained using isotype-matched antibodies as controls.

Cell suspensions from tumors were isolated as described above, then stained for viability using the Aqua Amine fixable live dead dye (Thermo Fisher Scientific) in PBS at room temperature using standard protocols, then cells were washed once, and resuspended in FACS buffer (PBS with 0.5% BSA and 2 mM EDTA) with Fc blocked using human TrueStain FcX (Biolegend) and incubated in the dark for 10 min at room temperature. For cell surface staining, cells were stained in 100 μL of FACS buffer and incubated for 30 minutes at 4C. For antigen-specific T cells CD8+ T cells, surface staining with an MC38 tumor antigen-derived peptide KSPWFTTL-H-2Kb tetramer was performed (KSPWFTTL-APC, MBL) (Lee, J. C. et al. Sci Immunol. 2020. 5. 10.1126/sciimmunol.aba0759). For intracellular Foxp3 staining, and additional step was performed using the Foxp3/Transcription Factor Staining Buffer Set (cat. no. 00-5523, eBioscience™) and FOXP3-BV421 or FOXP3-PE (Clone 150D, Biolegend) according to manufacture instructions. Cell populations were defined by the following markers: macrophages: CD45+, NK1.1−, Ly6C−, Ly6G−, CD11b+, F4/80+; monocytes: CD45+, NK1.1−, CD11b+, Ly6C+, Ly6G−, F4/80−; neutrophils: CD45+, NK1.1−, CD11b+, Ly6G+, Ly6Clow/−, F4/80−; DCs: CD45+, NK1.1−, F4/80−, CD11c+, MHC−II+; NK cells: CD45+, NK1.1+; CD8 T cells: CD45+, CD3+, CD8+, CD4−; Tetramer+CD8 T cells: CD45+, CD3+, CD8+, KSPWFTTL−H−2Kb+, CD4−; Terminally exhausted CD8 T cells: CD45+, CD3+, CD8+, CD4−, TCF1−, TIM3+; CD4 T cells: CD45+, CD3+, CD4+, CD8−, Foxp3−; Tregs: CD45+, CD3+, CD4+, CD8−, Foxp3+. For quantification of absolute numbers of cells, a defined number of fluorescent beads (Catalog C36950, ThermoFisher) was added to a known volume in each sample before acquisition and used as a counting reference. Then the absolute cell count was normalized to tumor weight (grams). Samples were analyzed using an Attune NxT flow cytometer (Thermo Fisher) and data was analyzed using FCS Express 7 Research.

### Example 1H. Hematological Analysis

50-100 μL blood samples were obtained from the retro-orbital sinus of mice under isoflurane anesthesia. Blood was placed in BD Microtainer tubes coated with ethylene diaminetetraacetic acid (K2-EDTA) and peripheral blood cell (RBC and platelets) counts were measured by Element HT5 hematology analyzer (Heska).

### Example 1I. Immunohistochemistry (IHC)

Formalin-fixed paraffin-embedded tumors of fresh frozen samples were prepared for immunostaining for F4/80 and CD11b. IHC was performed under standard procedures (laboratory of comparative pathology at Memorial Sloan Kettering Cancer Center), with either a primary ab (F4/80 and CD11b) or the ab dilution buffer as negative controls. The IHC samples were analyzed with a Nikon eclipse Ts2 microscope. To quantify the marker-positive cells, representative photographs of regions of interest (ROI) were taken at 20× by a blinded investigator, and Fiji (ImageJ) was used to analyze the percent of positive area for each the markers as previously described (Crowe, A. R. et al. Bio Protoc. 2019. 9. 10.21769/BioProtoc.3465).

### Example 1J. Human Monocyte-Derived Macrophages

Human peripheral blood mononuclear cells were collected from blood of healthy donors using density gradient medium (Lymphoprep™, Stemcell Technologies). CD14+ monocytes were positively selected to >95% purity by MACS using anti-CD14 microbeads (Miltenyi, Biotec), then plated at 1×10⁷/ml in 150×25 mm tissue culture plates in RPMI 1640 with 10% human serum and 1% pen-strep, and 50 ng/mL of recombinant human M-CSF (Biolegend), media was changed every 2 days. Differentiated macrophages were used after 7 days of maturation.

### Example 1K. Bone Marrow-Derived Macrophages

Bone marrow cells from the tibia and femurs of B6 mice were flushed using a syringe into DMEM (Life Technologies) supplemented with 10% fetal bovine serum (Life Technologies), 100 U/mL of penicillin, and 100 μg/mL of streptomycin (Life Technologies). Cells were centrifuged followed by RBC lysis (Biolegend) for 5 min, quenched with complete media, and filtered through a 70-uM cell strainer. Cells were centrifugated and resuspended in media containing 10 ng/mL macrophage-colony stimulating factor (M-CSF) (Peprotech) and plated on 10-cm untreated petri dishes per mouse in 15 mL of media and cultured for 7 days without replenishing or changing media to derive bone marrow derived macrophages. For polarization to Ml macrophages, cells were washed with complete media on day 8 and were cultured in 15 mL of new media with 10 ng/mL of M-CSF, 90 ng/ml of IFN-γ (Peprotech) and 10 ng/mL of LPS for 24 hours. For polarization to M2 macrophages, cells were washed with complete media on day 8 and were cultured in 15 mL of new media with 10 ng/ml of M-CSF, 90 ng/ml of IL-4 (Peprotech) and 400 ng/ml of IL-13 (Peprotech) for 24 hours.

### Example 1L. In Vitro Antibody-Dependent Macrophage Phagocytosis (ADCP)

To quantify ADCP, tumor cells were stained with carboxyfluorescein succinimidyl ester (CFSE), washed with serum-free DMEM, and plated at a density of 150,000 cells per well in 150 μL serum-free DMEM in a 96-well ultra-low attachment round-bottom plate (Cat. no. 7007; Costar) on ice. Tumor cells were opsonized by addition of 10 μL/mL of the various Fc variants of CD47 antibodies or control for 30 min on ice. Macrophages (bone marrow derived macrophages or human monocyte derived macrophages) were harvested by cell scraping, pelleted, washed in serum-free DMEM, and added to opsonized tumor cells at a density of 50,000 cells per well in 50 μL of media for a final assay volume of 200 μL and an effector-to-tumor cell ratio of 1:3. Cells were incubated at 37° C. for 2 h, pelleted, washed FACS buffer and stained with a 1:100 dilution of anti-mF4/80 or anti-m/hCD11b ab (Biolegend) FACS buffer for 30 min at 4° C. Cells were pelleted, washed with TrypLE and FACS buffer, and analyzed by flow cytometry using an Attune NxT flow cytometer (Thermo Fisher) and data was analyzed using FCS Express 7 Research. Phagocytosis was determined by the percentage of CFSE+ cells within the macrophage cell gate (CD11b+F480+ for mouse bone marrow derived macrophages and CD11b+CD14+ for human monocyte derived macrophages). Results are shown as fold change when compared to the level of phagocytosis in cells treated with control.

### Example 1M. Quantification and Statistical Analysis

Data was analyzed using Prism GraphPad software. One-Way ANOVA with Tukey's multiple comparison test was used to compare 3 or more groups. Unpaired 2-tailed t test was used when two groups were compared. All data, unless otherwise indicated, are plotted as mean±SEM. For statistical test, P values of ≤0.05 were statistically significant, indicated as *p<0.05, **p<0.01, ***p<0.001, and ****p<0.0001, not significant values (p>0.05) are denoted as n.s. Lines associated with asterisks indicate groups compared for significance.

A summary of all key resources used in the experiments described herein is provided in Table 3.

TABLE 3
Key resources.

| | | |
|---|---|---|
| REAGENT or RESOURCE | SOURCE | IDENTIFIER |

Antibodies

| | | |
|---|---|---|
| PerCP anti-mouse CD45 antibody | Biolegend | Cat# 103130 RRID: AB_893343 |
| AF700 anti-mouse CD45 antibody | Biolegend | Cat# 103128 RRID: AB_493715 |
| PE-Cy7 anti-mouse NK1.1 | Biolegend | Cat# 108714 RRID: AB_389363 |
| BV510 anti-mouse Ly6C | Biolegend | Cat# 128033 RRID: AB_2562351 |
| BV711 anti-mouse Ly6G | Biolegend | Cat# 127643 RRID: AB_2565971 |
| BV650 anti-mouse F4/80 | Biolegend | Cat# 123149 RRID: AB_2564589 |
| PE-Cy7 anti-mouse CD11c | Biolegend | Cat# 117318 RRID: AB_2493569 |
| APC-eFluor 780 anti-mouse MHCII | Invitrogen | Cat# 47-5321-82 RRID: AB_1548783 |
| Percp/cy5.5 anti-mouse CD3 | BioLegend | Cat# 100218 RRID: AB_1595492 |
| APC anti-mouse CD8 | BioLegend | Cat# 100712 RRID: AB_312751 |
| BV786 anti-mouse CD4 | BioLegend | Cat# 100453 RRID: AB_2565843 |
| BV650 anti-mouse/human CD11b | BioLegend | Cat# 101239 RRID: AB_11125575 |
| Alexa Fluor 647 anti-human/mouse | BioLegend | Cat# 515406 RRID: AB_2294995 |
| granzyme B | | |
| APC-Cy7 anti-mouse TCR b chain | BioLegend | Cat# 109220 RRID: AB_893624 |
| Pacific blue anti-mouse TCF1/TCF7 | Cell Signaling | Cat# 9066S RRID: AB_2797696 |
| BV605 anti-mouse TIM3 | BioLegend | Cat# 119721 RRID: AB_2616907 |
| BV711 PD-1 | BioLegend | Cat# 135231, RRID: AB_2566158 |
| PE-Cy7 Ki67 | Thermo Fisher | Cat# 25-5698-82, RRID: AB_11220070 |
| PE anti-mouse FOXP3 | BioLegend | Cat# 320008, RRID: AB_492980 |
| Fc block, TruStain FcX | BioLegend | Cat# 101320, RRID: AB_1574975) |
| PE anti-mouse CD47 (MIAP301) | BioLegend | Cat# 127507 RRID: AB_1134133 |
| APC anti-human CD47 (CC2C6) | Thermo Fisher | Cat# 17-0479-41, RRID:AB_1963587 |
| BV605 anti-mouse SIRPa | BD Biosciences | Cat# 740390, RRID: AB_2740120 |
| APC anti-human SIRPa | BioLegend | Cat# 372105, RRID: AB_2650862 |
| BV605 anti-human CD64 | BioLegend | Cat# 305034, RRID: AB_2566237 |
| PE anti-human CD16 | BioLegend | Cat# 980102, RRID: AB_2616616 |
| AF700 anti-human CD14 | BioLegend | Cat# 301822, RRID: AB_493747 |
| In VivoMAb anti-mouse CD8a | BioXcell | Cat# BE0061, RRID: AB_1125541 |
| Peroxidase conjugated | Jackson | Cat# 109-036-088, RRID: AB_2337594 |
| anti-human IgG | IummunoResearch | |
| Peroxidase conjugated | Jackson | Cat# 115-036-146, RRID: AB_2307347 |
| anti-mouse IgG | IummunoResearch | |
| Recombinant anti-CD11b | Abcam | Cat# ab133357, RRID: AB_2650514 |
| Recombinant anti-F4/80 | Abcam | Cat# ab300421, RRID: AB_2936298 |
| MIAP301 mIgG1-D265A | Developed herein | N/A |
| Recombinant anti-CD11b | Abcam | Cat# ab133357, RRID: AB_2650514 |
| Recombinant anti-F4/80 | Abcam | Cat# ab300421, RRID: AB_2936298 |
| MIAP301 mIgG1 | Developed herein | N/A |
| MIAP301 mIgG2a | Developed herein | N/A |
| 2D3 mIgG1 mIgG2a | Developed herein | N/A |
| 5F9 mIgG1-D265A | Developed herein | N/A |
| 5F9 mIgG1 | Developed herein | N/A |
| 5F9 mIg2a | Developed herein | N/A |
| 5F9-hIgG4 | Developed herein | N/A |
| 5F9-hIgG1 | Developed herein | N/A |
| 5F9-hIgG1-GAALIE | Developed herein | N/A |
| Anti-mouse PD-1 mIgG1-D265A | Dahan et al., Cancer | N/A |
| | Cell 2015 | |
| Anti-gp75-mIgG2a | Weitzenfeld et al., J | N/A |
| | Clin Invest 2019 | |
| Anti-CCR2 antibody | Mack et al., J | N/A |
| | Immunol 2001 | |
| Dylight650 anti-human CD32B | Smith et al., | N/A |
| | PNAS 2012 | |
| Dylight488 anti-human CD32A | Smith et al., | N/A |
| | PNAS 2012 | |

Chemicals, peptides, and recombinant proteins

| | | |
|---|---|---|
| Recombinant mouse CD47 | SinoBiological | Cat# 57231-MNAH |
| Recombinant human SIRPα-hFc | SinoBiological | Cat# 11612-H02H1 |
| Recombinant human CD47 | SinoBiological | Cat# 12283-HCCH |
| clodronate liposomes with | Formumax Scientific | Cat# F70101C-2 |
| control liposomes | Inc | |
| Aqua Amine fixable live dead dye | ThermoFisher | Cat# L34965 |
| Absolute counting beads | ThermoFisher | Cat# C36950 |
| Recombinant mouse M-CSF | Peprotech | Cat# 315-02 |
| Recombinant mouse IFN-g | Peprotech | Cat# 315-05 |
| Recombinant mouse IL-4 | Peprotech | Cat# 214-14 |
| Recombinant mouse IL-13 | Peprotech | Cat# 210-13 |
| Recombinant human M-CSF | Biolegend | Cat# AF-300-25 |
| Recombinant human IFN-g | Peprotech | Cat# 300-02 |
| Recombinant human IL-4 | Peprotech | Cat# 200-04 |
| CellTrace ™ CFSE Cell | ThermoFisher | Cat# C34554 |
| Proliferation Kit | | |
| CD14 MicroBreads, Human | Miltenyi Biotec | Cat# 130-050-201 |
| H-2kb MuLV p15E Tetramer- | BML | Cat# TB-M507-2 |
| KSPWFTTL-APC | | |

Commercial assays

| | | |
|---|---|---|
| Tumor tissue dissociation | Miltenyi Biotec | Cat# 130-096-730m |
| Foxp3/Transcription Factor | eBioscienceTM | Cat# 00-5523 |
| Staining Buffer Set | | |

Experimental models: Cell lines

| | | |
|---|---|---|
| MC-38 | Kerafast | Cat# ENH204-FP, RRID: CVCL_B288 |
| B16-F10 | ATCC | Cat# CRL-6475, RRID: CVCL_0159 |
| LL/2 (LLC1) | ATCC | Cat# CRL-1642, RRID: CVCL_4358 |
| Jurkat | ATCC | Cat# TIB-152, RRID: CVCL_0367 |
| Raj | ATCC | Cat# CCL-86, RRID: CVCL_0511 |
| HKP1 | Markowitz et al., JCI | N/A |
| | insight 2018 | |
| MC38 hCD47 KI | Developed herein | N/A |
| B16 hCD47 KI | Developed herein | N/A |

Experimental models: Organisms/strains

| | | |
|---|---|---|
| C57BL/6J (WT) | Jackson | Strain #:000664, RRID: |
| | | IMSR_JAX: 000664 |
| FcR-γ chain^{—/—} | Takai et al., | N/A |
| | Cell 1994 | |
| Mice humanized for FcγRs | Smith et al., | N/A |
| | PNAS 2012 | |
| hCD47/hSIRPα KI | Developed herein | N/A |
| hCD47/hSIRPα/hFcγR | Developed herein | N/A |

Software and algorithms

| | | |
|---|---|---|
| FCS Express 7 Research | Denovo software | denovosoftware.com/ |
| ImageJ | Schneider et al. | imagej.net/software/imagej/ |
| | Nat Methods 2012 | |

Other

| | | |
|---|---|---|
| Attune NxT flow cytometer | Thermo Fisher | N/A |
| Element HT5 hematology | Heska | HT5 |
| analyzer | | |
| NanoDrop 2000 | Thermo | ND2000 |
| spectrophotometer | | |

### Example 2. Engagement of Activating FcγRs Augments the In Vivo Antitumor Activity of Anti-mCD47 Antibodies

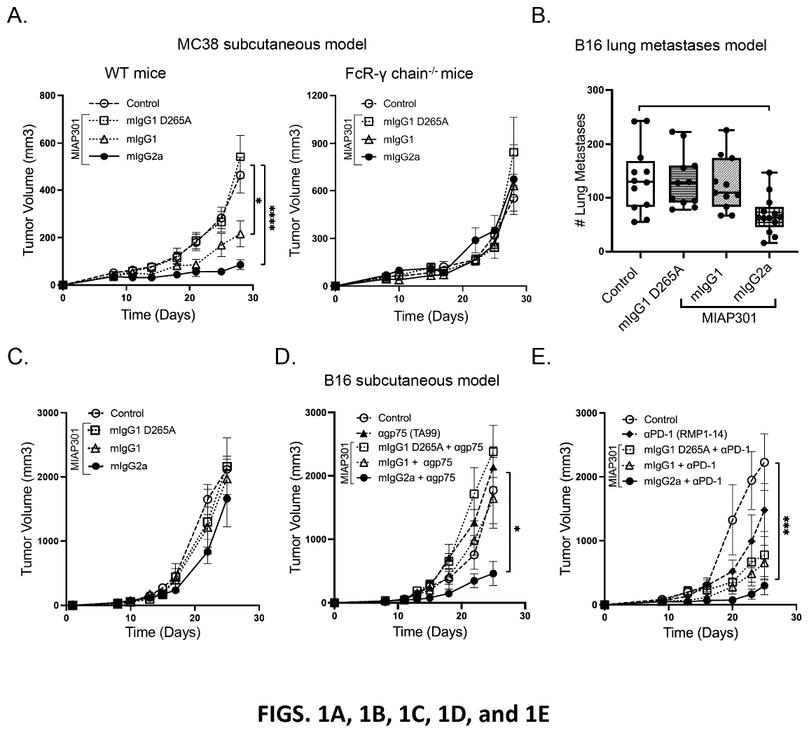

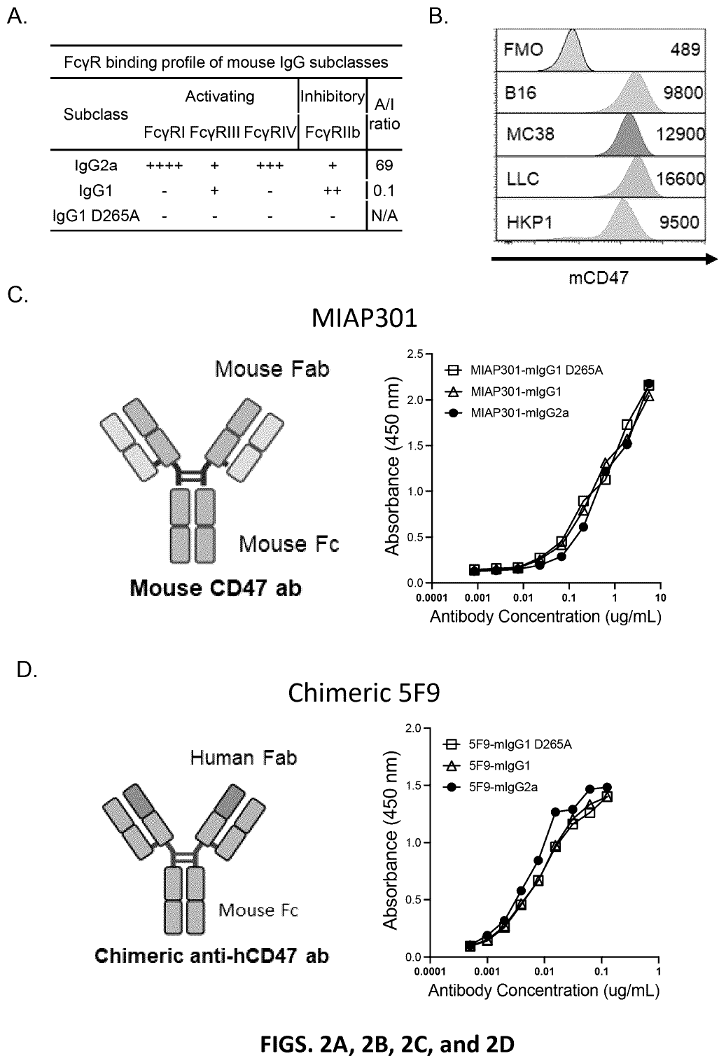

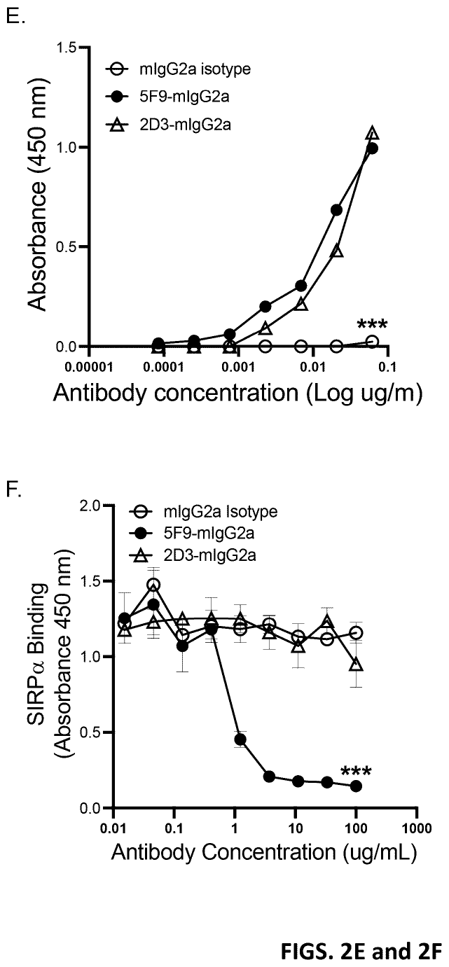

To determine the contribution of Fc-FcγR interactions to the in vivo activity of antibodies targeting the CD47-SIRPα axis, the Fc domain of the anti-mCD47 ab clone MIAP301 was modified to generate antibodies with varying affinity to mFcγRs: 1) mIgG2a Fc, binding preferentially to activating mFcγRs I and IV (Activating to inhibitory [A/I] ratio=69), 2) mIgG1 Fc, binding preferentially to the inhibitory mFcγRIIB and to a less extent mFcγRIII (A/I ratio=0.1), and 3) mIgG1-D265A Fc, which lacks binding to any mFcγRs (FIG. 2A) (Nimmerjahn, F. et al. Science. 2005. 310, 1510-1512). Alterations to the Fc domain of MIAP301 did not alter the binding specificity of the variable regions to mCD47 (FIG. 2C), nor its in vivo half-life (Dahan, R. et al. Cancer Cell. 2015 28, 543), which allows comparisons into the contributions of differential FcγR engagement to the activity of CD47 blocking antibodies. The in vivo antitumor activity of these different Fc subclasses of MIAP301 were compared in wild type B6 (WT) mice bearing subcutaneous MC38 colorectal adenocarcinomas, a tumor type expressing high levels of CD47 (FIG. 2B). The mIgG2a Fc variant of MIAP301 led to the most significant reduction in tumor burden when compared to the control or other Fc variants (FIG. 1A [left]). Of note, this therapeutic effect was attenuated for the mIgG1-D265A Fc variant and in mice lacking all activating FcγRs (FcR-γ chain−/−) 42 (FIG. 1A [right]). The mIgG2a Fc variant of MIAP301 also demonstrated the most significant decrease in the number of lung metastases in a second tumor model using B16 melanoma cells (FIG. 1B).

Currently, there are several clinical trials investigating anti-CD47 antibodies in combination with therapies blocking tumor antigens of immune checkpoints (Maute, R. et al. Immunooncol Technol. 2022. 13, 100070). Therefore, the anti-tumor activity of the different Fc variants of MIAP301 was compared in combination with a tumor targeting ab against gp75 (Clone TA99, mIgG2a ab) 44 or with an ab blocking the immune checkpoint PD-1 (Clone RMP1-14, mIgG1 D265A Fc). To assess these combinations, a subcutaneous B16 melanoma model was used in which none of the MIAP301 Fc variants worked as monotherapy (FIG. 1C). The combination of anti-gp75 ab with the MIAP301-mIgG2a ab led to significant reduction in tumor burden when compared to anti-gp75 ab alone or the other combinations (FIG. 1D). Similarly, combination of the anti-PD-1 ab with MIAP301-mIgG2a ab significantly decreased tumor burden when compared to PD-1 blockade alone or the other combinations (FIG. 1E). The enhanced activity of the mIgG2a ab compared to the other Fc subclasses alone or in combination with tumor targeting antibodies or immune checkpoint blockade indicates that engagement of activating FcγRs by CD47 antibodies contributes to its optimal in vivo anti-tumor activity.

### Example 3. Engagement of Activating FcγRs During Treatment with Anti-CD47 Antibodies Correlates with Increased Number of Tumor-Infiltrating Macrophages and Enhanced Phagocytic Activity

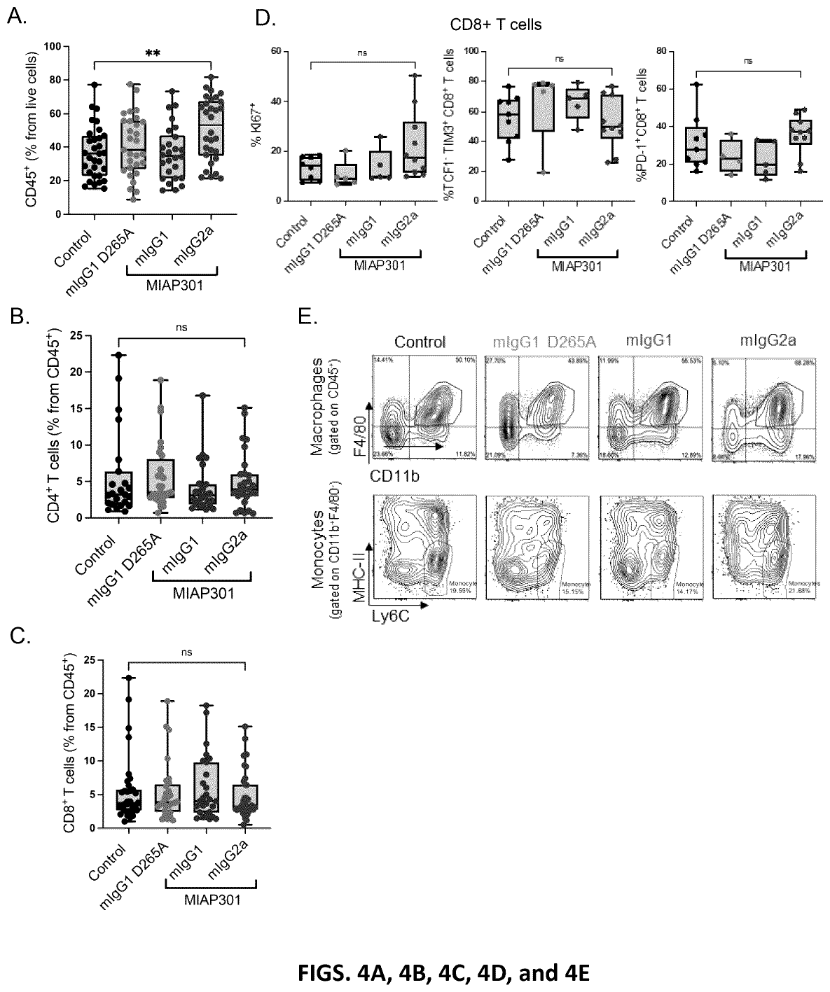

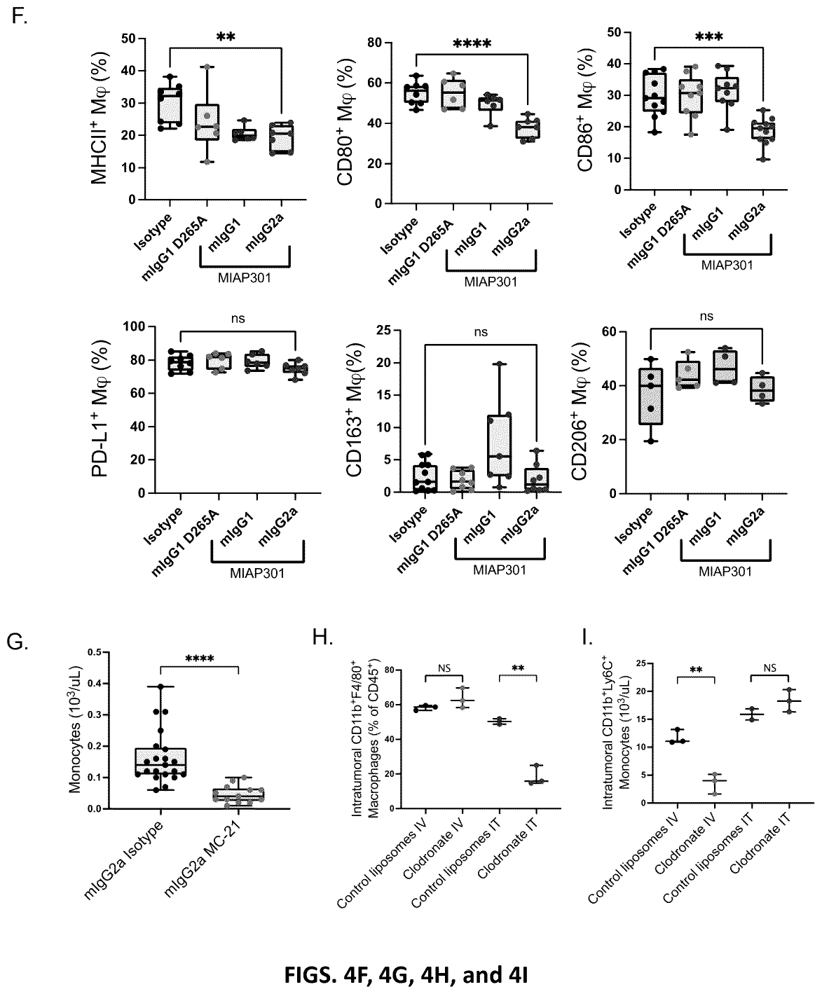

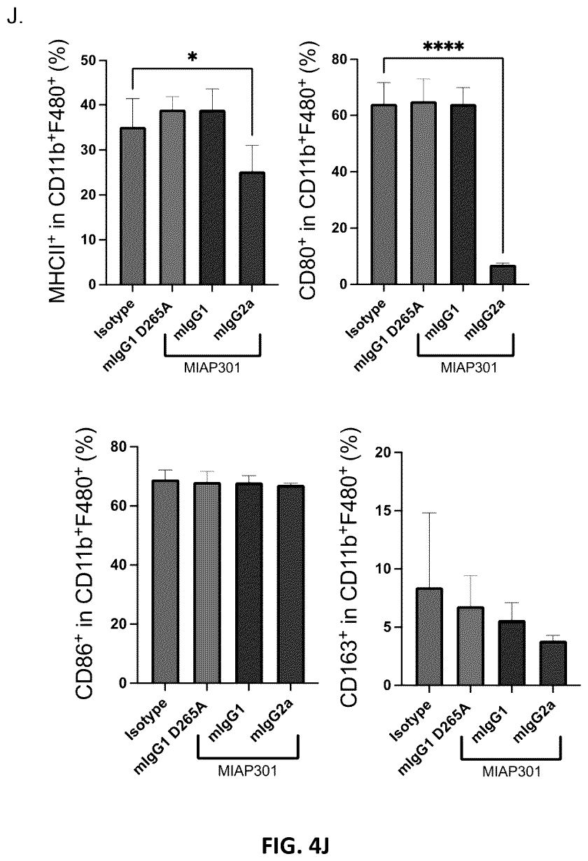

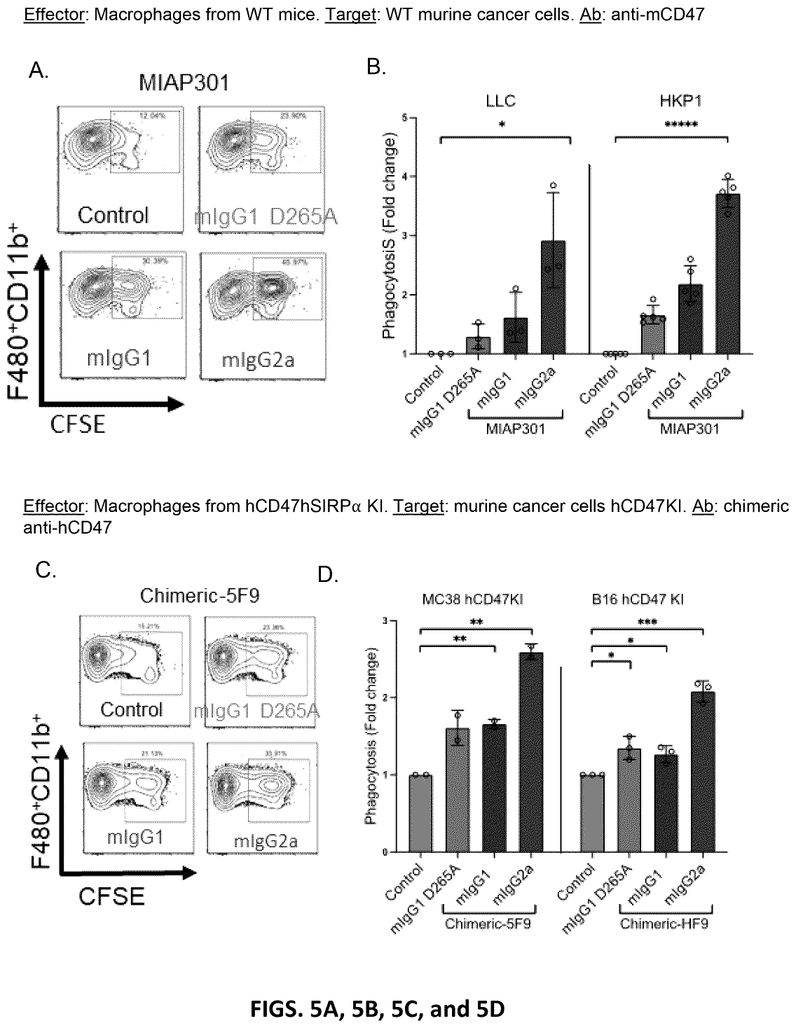

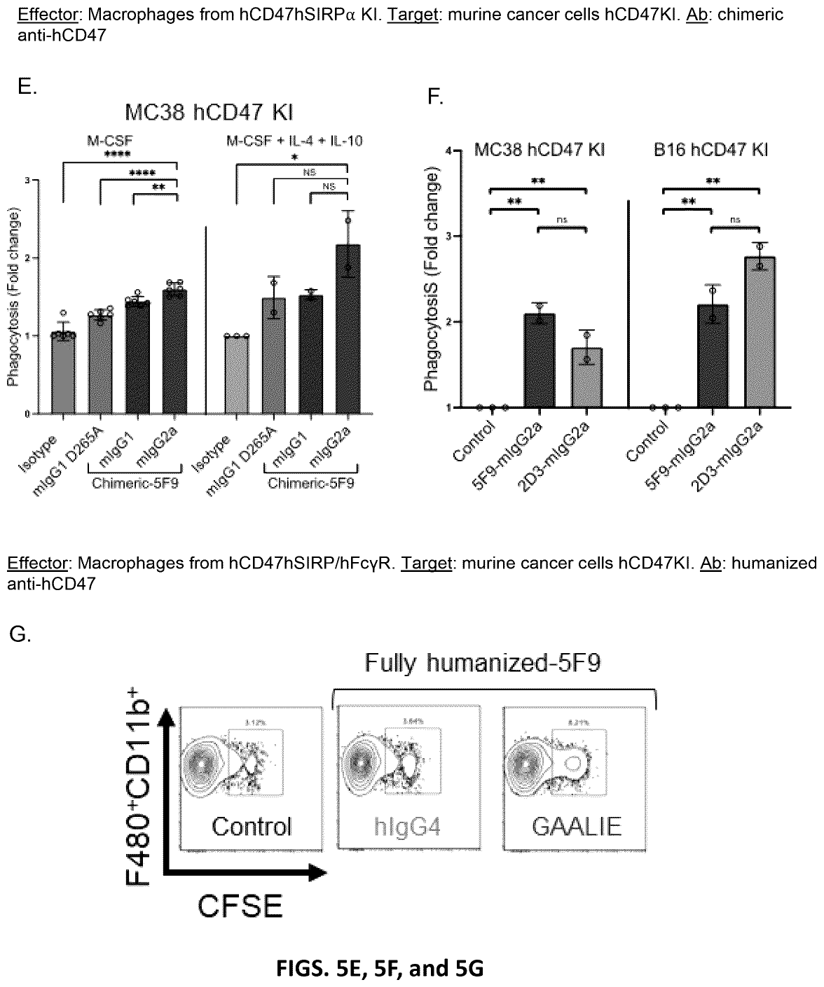

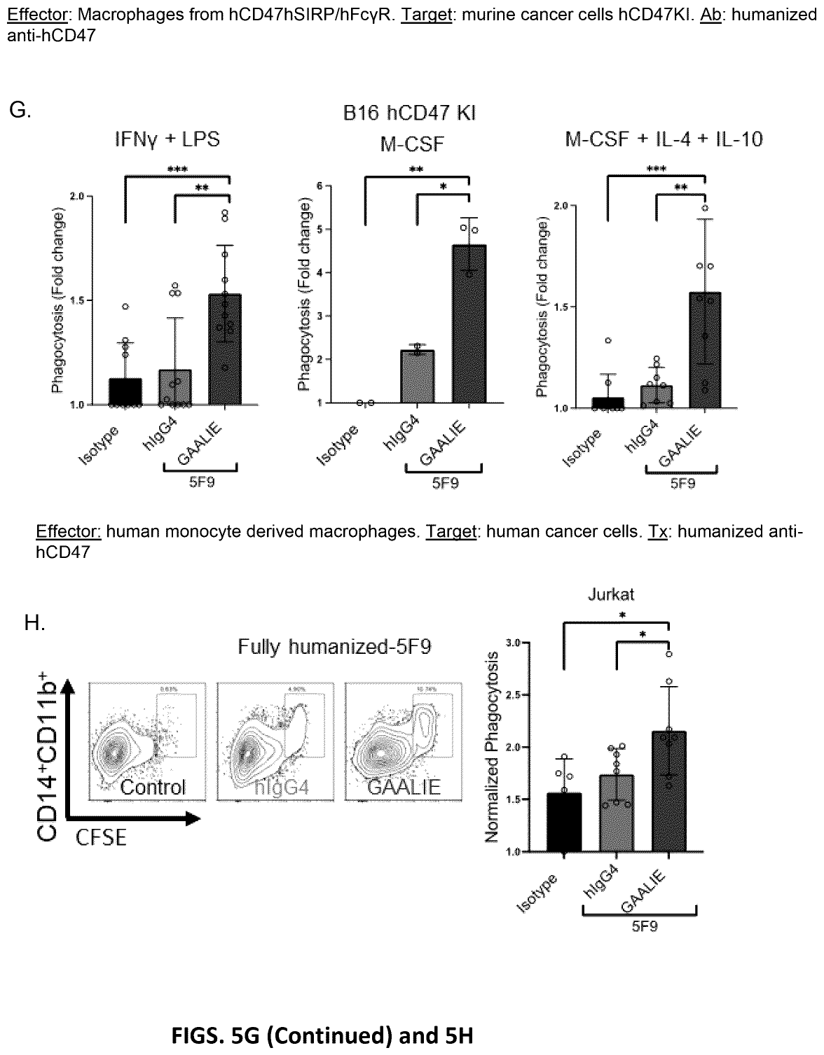

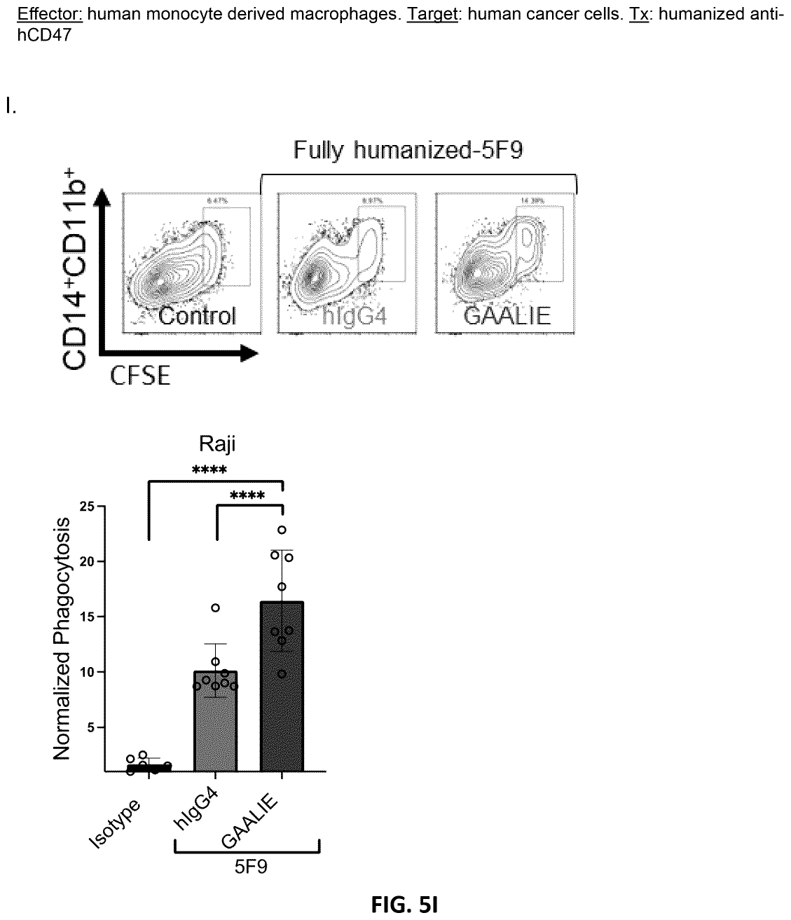

To evaluate the factors that promote the enhanced activity of the mIgG2a Fc subclass of MIAP301, the effect of the three Fc variants was compared on myeloid cells in the TME. Treatment with the MIAP301-mIgG2a ab correlated with a significant increase in the percentage of CD45⁺ leukocytes (FIG. 4A), mainly attributed to a significant increase in CD11b⁺F4/80⁺ macrophages and CD11b⁺Ly6C⁺ monocytes, but not neutrophils or dendritic cells (FIGS. 3A and 4E). Phenotypic analysis revealed decreased levels of MHCII, CD80 and CD86 in tumor infiltrating macrophages. However, there was no significant upregulation of markers associated with an immunosuppressive phenotype (CD206, CD163 or PD-L1) after treatment with MIAP301-mIgG2a (FIG. 4F). Remarkably, treatment with MIAP301-mIgG2a ab led to increased localization of macrophages into the tumor core when compared to control or other Fc-variants, in which a significant proportion of macrophages remained located in the periphery of the tumor bed (FIG. 3B), suggesting that MIAP301-mIgG2a ab led to increased infiltration and colocalization of macrophages with tumor cells. These findings suggested that macrophages and/or monocytes could mediate the effector response during treatment with this Fc variant. To test this hypothesis, tumor infiltrating macrophages or CCR2⁺ monocytes were selectively depleted using clodronate or an anti-CCR2 ab (mIgG2a), respectively (FIGS. 4G-4I). Depletion with clodronate-but not with anti-CCR2 ab-abrogated the activity of the MIAP301-mIgG2a ab (FIGS. 3C-3D), indicating that macrophages are required effector cells for the increased therapeutic activity of MIAP301-mIgG2a. Since CD47 constitutes a well-known antiphagocytic signal on macrophages, the effect of the different anti-mCD47 Fc subclasses in macrophage activity was assessed by performing in vitro phagocytosis assays of MC38 and B16 cancer cells using bone marrow-derived macrophages. The MIAP301-mIgG2a ab led to significantly more phagocytosis of both cancer cell lines when compared to control or the other Fc subclasses (FIGS. 3E and 5A). These effects were also seen when other murine cancer cells lines were used as targets (FIG. 5B). Similar to the results observed in vivo, enhanced phagocytosis of cancer cells by MIAP301-mIgG2a resulted in downregulation of MHCII, CD80 and CD86 without increasing levels of immunosuppressive markers (FIG. 4J), suggesting that these activation markers are downregulated as a response to the enhanced phagocytic activity during treatment with MIAP-mIgG2a, rather than directly reprograming macrophages towards an immunosuppressive state. Collectively, these results indicate that engagement of activating FcγRs by CD47 antibodies increases the number of tumor-infiltrating macrophages and enhances their tumor phagocytic activity.

### Example 4. Engagement of Activating FcγRs During Treatment with Anti-CD47 Antibodies Depletes Regulatory T Cells (T_{regs}), and Promotes Antigen-Specific T Cell Responses and Long-Term Antitumor Immunity

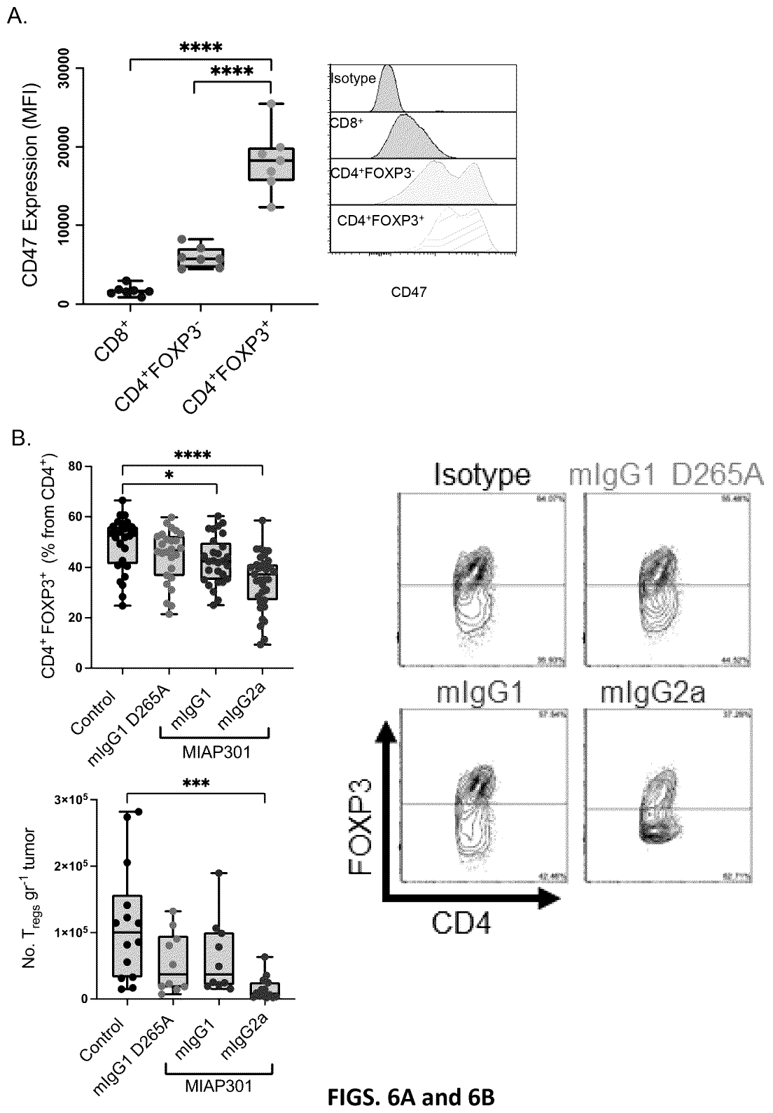

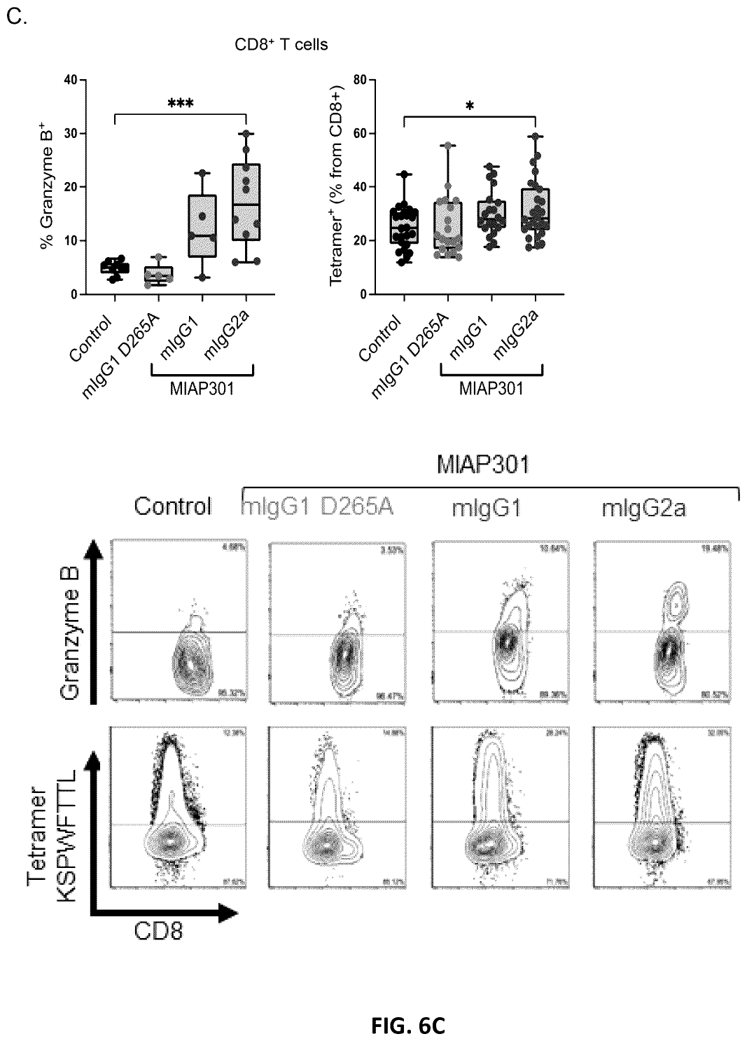

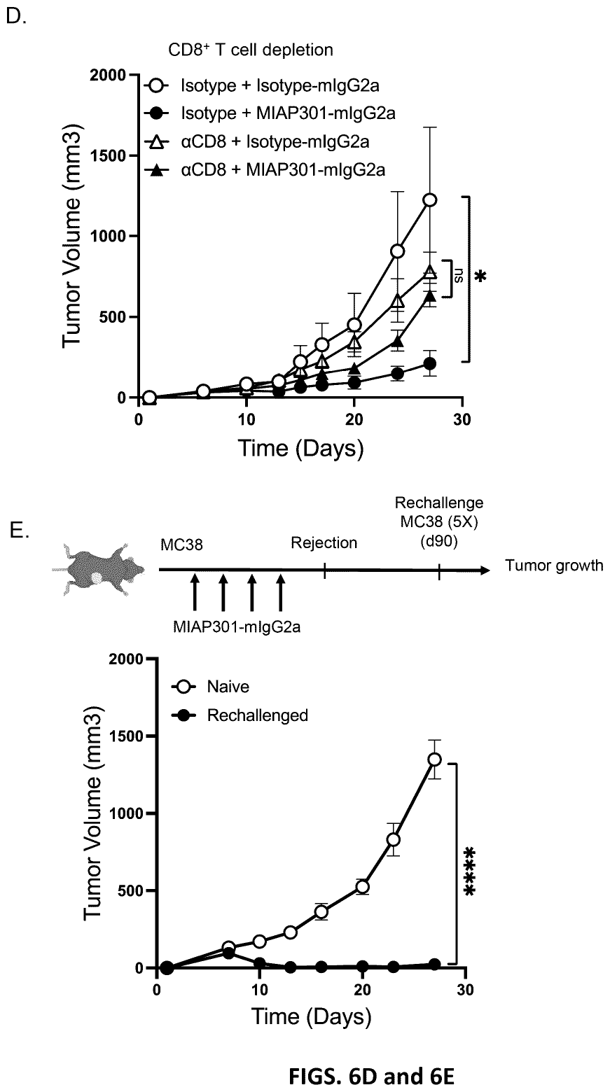

CD47 blockade promotes adaptive immune responses by increasing cross-presentation of tumor antigens (Liu, X. et al. Nat Med. 2015 21, 1209-1215 and Tseng, D. et al. PNAS. 2013. 110, 11103-11108) and by inducing type 1 immune responses through direct ligation of CD47 expressed on T cells (Bouguermouh, S. et al. J Immunol. 2008. 180, 8073-8082). The patterns of CD47 expression on CD8⁺, CD4⁺FOXP3⁻, and CD4⁺FOXP3⁺ (T_{regs}) T cells in the TME were analyzed first. It was determined that all three T cell subsets expressed CD47, however T_{regs} exhibited a significantly higher expression when compared to CD4⁺FOXP3⁻ or CD8⁺ T cells (FIG. 6A). This differential expression of CD47 supports the possibility that the anti-tumor effect observed after treatment with an Fc-enhanced anti-CD47 ab results from both enhanced phagocytosis and depletion of T_{regs} in the TME. To test this hypothesis, the effect of the three sub-classes of MIAP301 on T_{regs} in the TME of MC38 tumors was assessed. Treatment with the MIAP301-mIgG2a ab led to a significant decrease in the percentage and absolute number of CD4⁺FOXP3⁺ T cells when compared to control tumors (FIG. 5B), suggesting that treatment with the MIAP301-mIgG2a ab is associated with an Fc-mediated decrease in the frequencies of immunosuppressive CD47^{high} expressing T_{regs}.

Since T_{regs} can restrain effective adaptive antitumor activity (Shan, F. et al. Therapeutic targeting of regulatory T cells in cancer. Trends Cancer. 2022. 8, 944-961), it was investigated whether decreased levels of T_{regs} correlate with a higher number of CD4⁺FOXP3⁻ or CD8⁺ T cells. While no significant changes were seen in the percentages of these two populations (FIGS. 4B-4C), CD8⁺ T cells derived from MIAP301-mIgG2a treated tumors displayed a significantly increase in Granzyme B production and a trend towards increased levels of Ki67 (FIGS. 6C [left] and 4D). Additionally, a substantial rise in antigen-specific T cells was seen in these tumors (FIG. 6C [right]). Notably this increase in CD8⁺ T cell activation occurred without concurrent upregulation of cell surface markers associated with CD8⁺ T cell exhaustion (FIG. 4D). Furthermore, depletion of CD8⁺ T cells abrogated the antitumor activity of the MIAP301-mIgG2a ab, indicating that CD8⁺ T cells are required for its optimal therapeutic activity (FIG. 6D). To determine whether tumor-specific T cell responses induced by the MIAP301-mIgG2a ab resulted in long term immune memory, WT mice in which MIAP301-mIgG2a ab led to a complete antitumor response were subsequently rechallenged with a 5-fold higher dose of MC38 tumor cells 90 days following initial tumor challenge in the absence of additional therapy. Mice previously treated with the MIAP301-mIgG2a ab completely rejected the tumor rechallenge when compared to naïve mice (FIG. 6E). Overall, these findings indicate that treatment with a mIgG2a anti-CD47 ab facilitates long term CD8⁺ tumor-specific antitumor immunity.

### Example 5. Generation of CD47/SIRPα Humanized Knock-In (KI) Mice to Assess Antibodies Targeting hCD47 In Vivo

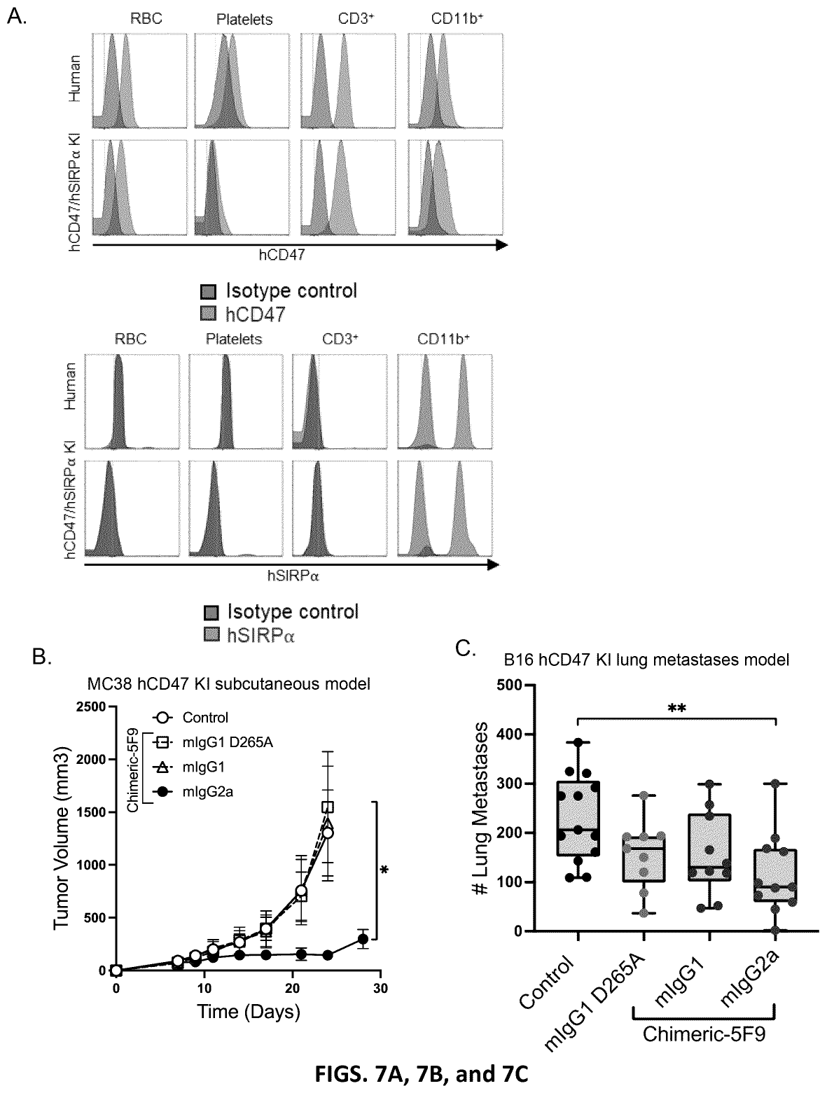

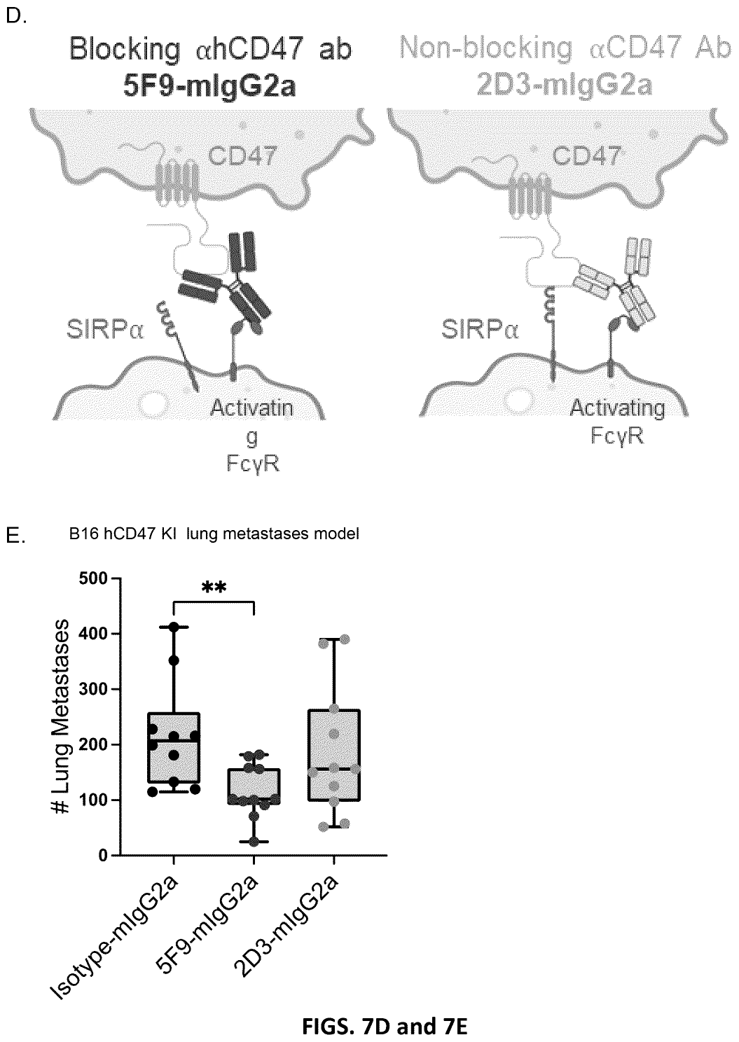

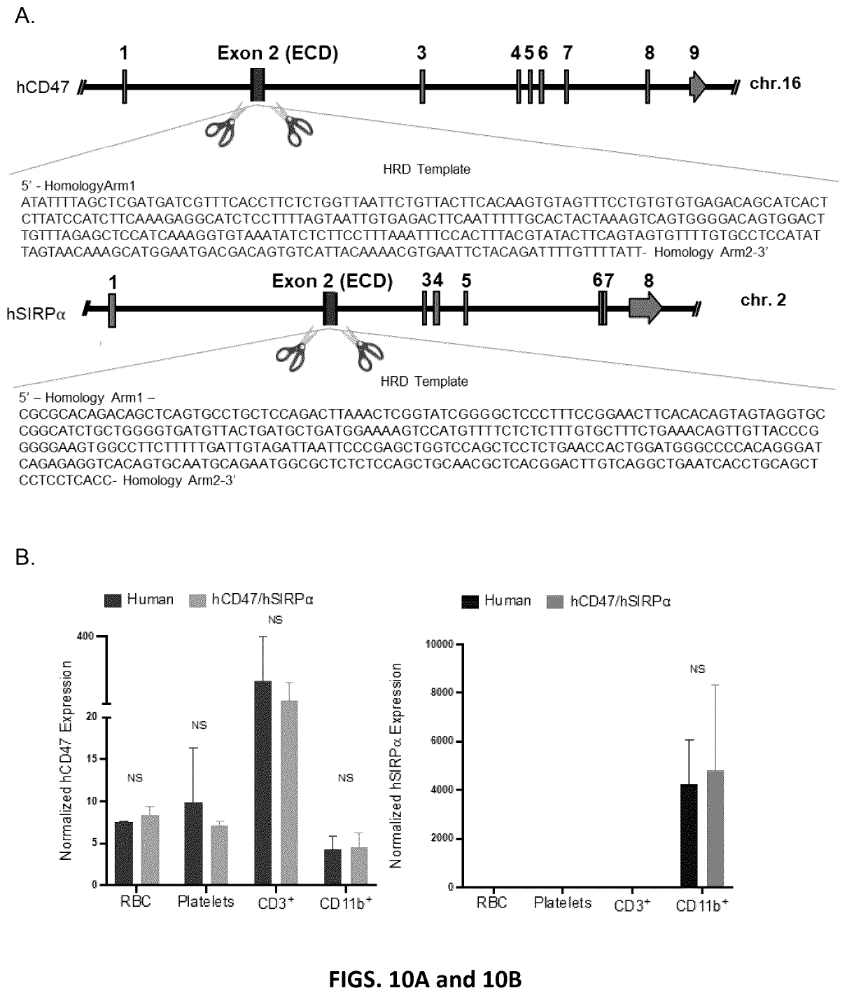

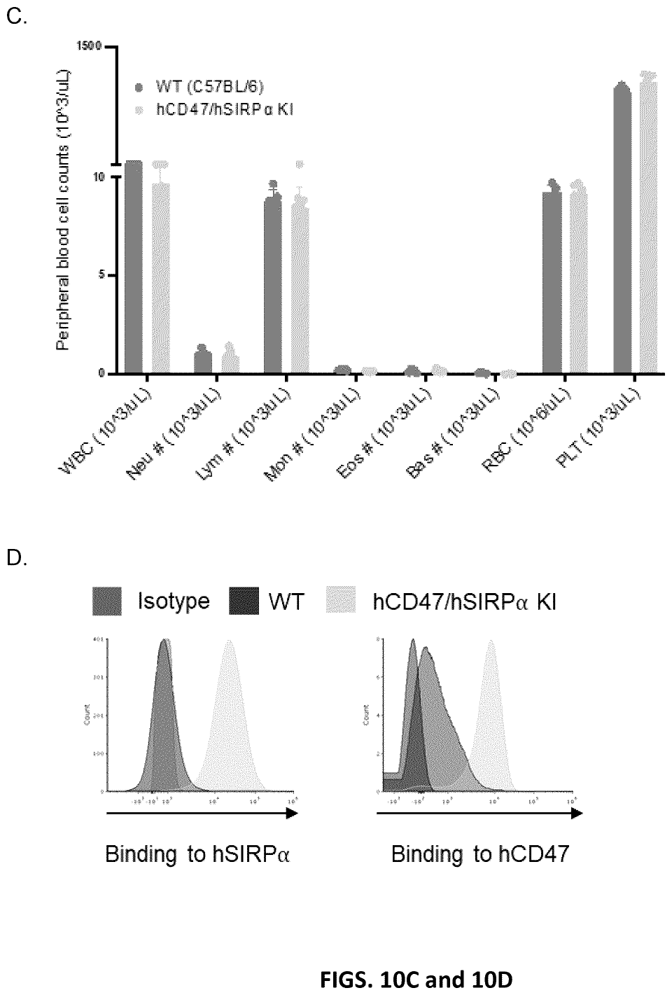

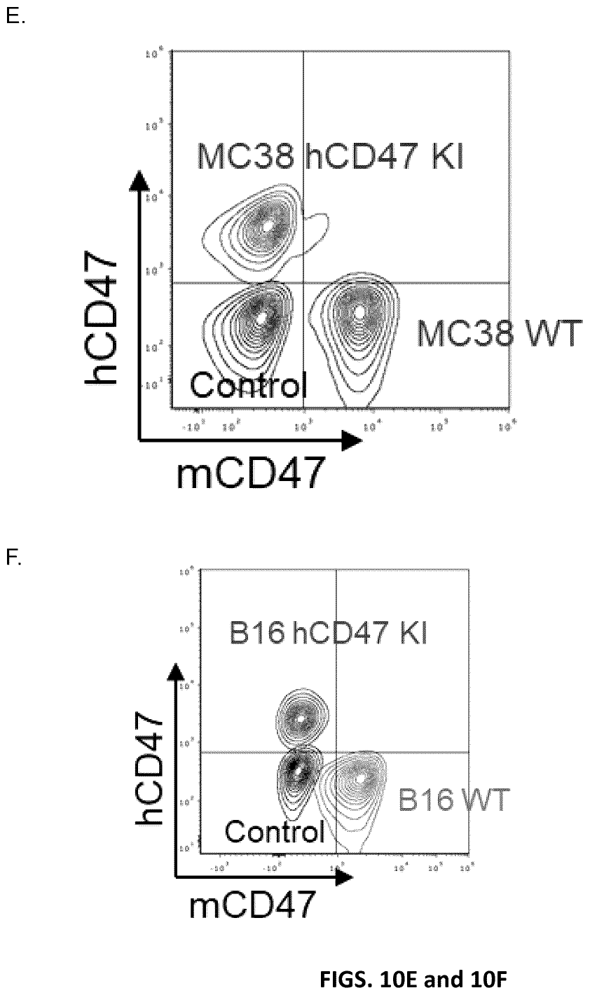

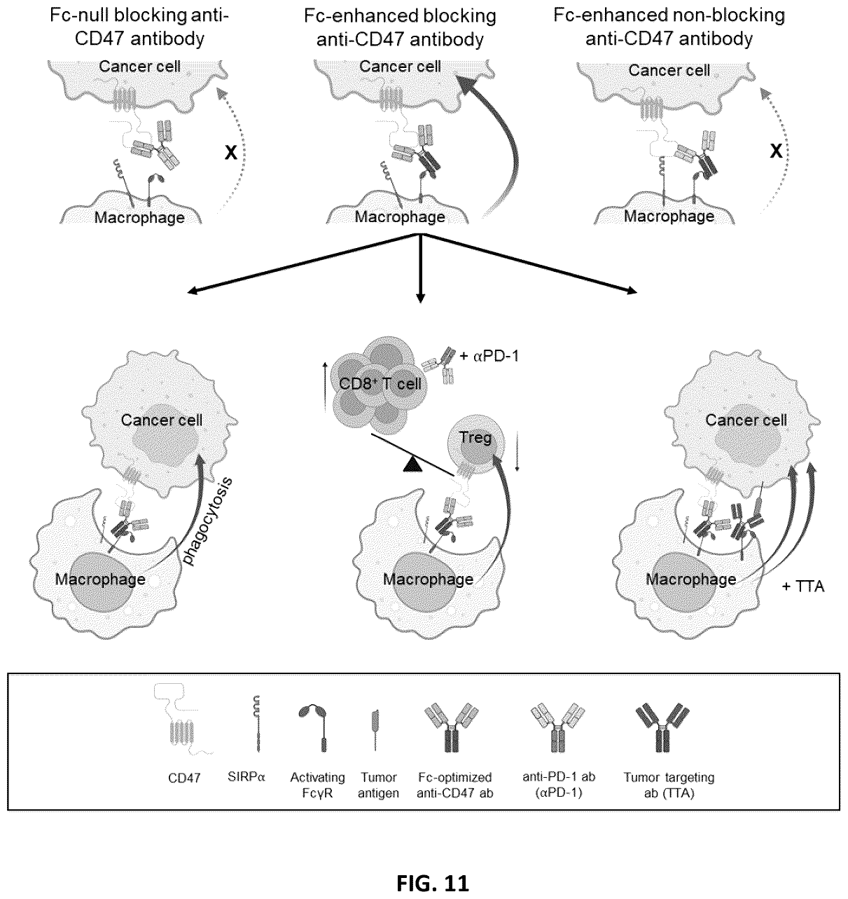

Having established the contribution of mFcγR engagement in an immune competent murine system, it was determined whether these findings were applicable for antibodies targeting hCD47. For this purpose, a humanized mouse strain was developed in which the human extracellular domains (ECD) of CD47 and SIRPα replaced their respective murine counterparts using CRISPR/Cas9 gene-editing (hCD47/hSIRPα KI mice) (FIG. 10A). The expression pattern of hCD47 and hSIRPα in these mice was assessed and it was found that, similar to the pattern seen on human cells, hCD47 was ubiquitously expressed on peripheral blood RBC, platelets, CD3⁺ and CD11b⁺ cells. In contrast, SIRPα was expressed primarily on CD11b⁺ cells, but not in other cells (FIG. 7A). No significant differences in the levels of expression of either hCD47 and hSIRPα were observed between blood obtained from humans or hCD47/hSIRPα KI mice (FIG. 10B). Peripheral blood levels of RBC, platelets and other leukocytes were similar than those found WT mice (FIG. 10C). Furthermore, the hCD47 and hSIRPα expressed on these humanized mice were capable of binding their human counterparts (FIG. 10D). In an analogous CRISPR/Cas9 gene-editing strategy, MC38 and B16 cancer cell lines were generated in which their mCD47 ECD was replaced by the hCD47 ECD (MC38-hCD47 KI, B16-hCD47 KI, respectively) (FIGS. 10E-10F). These cell lines can be engrafted in the hCD47/hSIRPα KI mouse system and represent a unique system to test anti-human antibodies.

To test the therapeutic activity of antibodies against hCD47, the 5F9 clone (Magrolimab) was used, which is currently being investigated in several clinical trials (Advani, R. et al. N Engl J Med. 2018. 379, 1711-1721). Chimeric antibodies were generated containing human Fab and the three mouse Fc subclasses with varying affinities to mFcγRs outlined before: 5F9-mIgG2a, 5F9-mIgG1, and 5F9-mIgG1-D265A ab (FIGS. 2A and 2D). Using hCD47/hSIRPα KI mice, the therapeutic activity was compared of the three Fc variants of 5F9 in the MC38 hCD47KI subcutaneous model and the B16 hCD47KI lung metastases model. Similar to the mIgG2a MIAP301 Fc variant, the 5F9-mIgG2a ab led to the most significant reduction in tumor burden in both tumor models when compared to control or other Fc variants (FIGS. 7B-7C). Furthermore, the 5F9-mIgG2a ab led to the most significant amount of in vitro phagocytosis of both cancer cells when compared to control or the other Fc subclasses (FIGS. 7C-7D). These effects were also seen under different in vitro macrophage polarization states (FIG. 5E). Altogether, these results validate prior findings in the immune competent murine backgrounds and further support the role of activating FcγRs in modulating the in vivo activity of hCD47 antibodies.

### Example 6. Effective In Vivo Antitumor Activity Requires Both Blockade of the CD47/SIRPα Axis and Engagement of Activating FcγR Signaling

Using a humanized system for CD47 antibodies, it was determined whether engagement of activating FcγRs is sufficient, or whether concomitant blockade of the CD47/SIRPα signal is also required to achieve optimal in vivo therapeutic activity. To address this question, the in vivo antitumor activity of two antibodies was compared targeting hCD47: The 5F9 ab clone which blocks the interaction between CD47 and SIRPα, and the 2D3 ab clone which binds to CD47, but does not block its interaction with SIRPα (Tseng, D. et al. PNAS. 2013. 110, 11103-11108) (FIG. 7D). Both antibodies were generated as mIgG2a Fc subclass to retain engagement of activating FcγRs, which enabled the investigation of the contribution of the Fab domains to the antitumor activity. It was confirmed that both 5F9-mIgG2 and 2D3-mIgG2a bound to hCD47 (FIG. 2E), but only 5F9-mIgG2a was able to block the interaction with hSIRPα (FIG. 2F). Furthermore, similar to the 5F9-mIgG2a ab, the 2D3-mIgG2a ab was capable of inducing in vitro ADCP of cancer cells, indicating its capacity to bind FcγRs (FIG. 5F). The therapeutic activity of the two antibodies was compared in the B16 hCD47 KI lung metastases model and found that only the 5F9-mIgG2a ab led to significantly decreased tumor burden when compared to the 2D3-mIgG2a ab or control (FIG. 7E). These results suggest that in order to achieve significant antitumor activity, anti-CD47 antibodies require both mechanisms of activity: blockade of the CD47/SIRPα (antiphagocytic) signal by the Fab domain, and engagement of the activating FcγRs (pro-phagocytic) signal by the Fc domain.

### Example 7. Mice Humanized for CD47, SIRPα and FcγR are a Reliable Platform for the Evaluation of Toxicity and Therapeutic Activity of Fully Human Anti-CD47 Antibodies

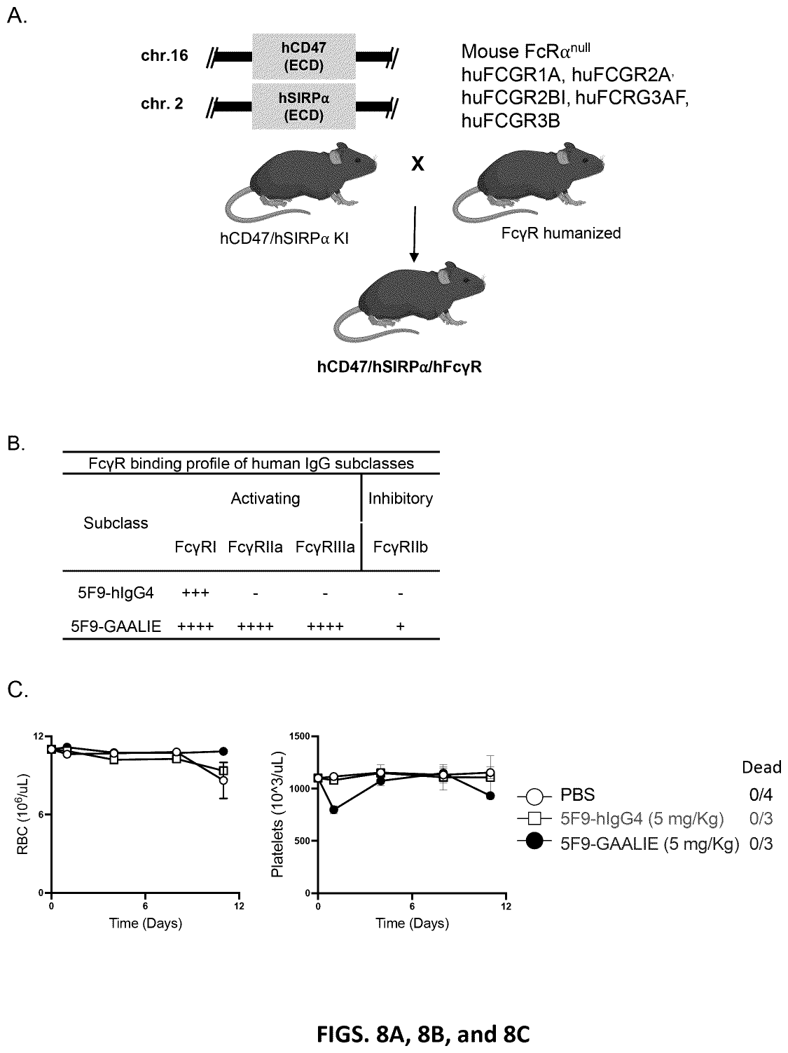

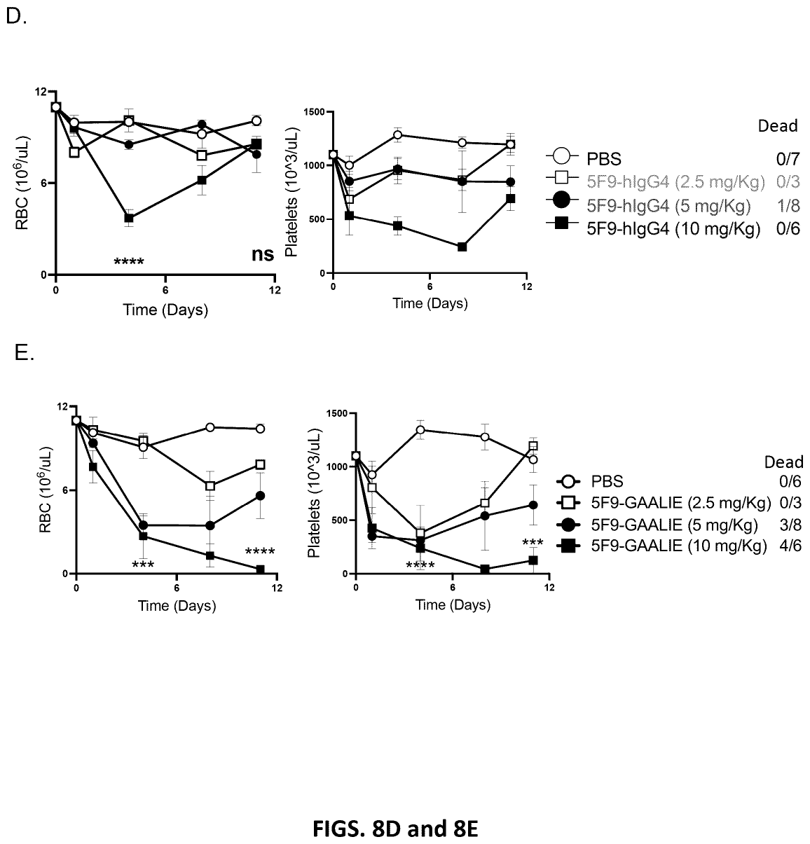

After demonstrating the biological rationale for engaging activating FcγRs in both murine and human chimeric anti-CD47 antibodies, the therapeutic potential of fully humanized anti-CD47 antibodies relevant for clinical translation was investigated. Currently, most anti-hCD47 antibodies translated into patients have been studied in xenograft, immunocompromised or purely murine backgrounds (Majeti, R. et al. Cell. 2009. 138, 286-299; Weiskopf, K. et al. J Clin Invest. 2016. 126, 2610-2620; Liu, X. et al. Nat Med. 2015. 21, 1209-1215; and Edris, B. et al. PNAS. 2012. 109, 6656-6661). Unfortunately, these models do not fully recapitulate the on-target off-tumor toxicity as well as therapeutic activity observed in patients receiving these therapies (Sikic, B. I. et al. J Clin Oncol. 2019. 37, 946-953 and Zeidan, A. M. et al. Ann Hematol. 2022. 101, 557-569). To overcome this limitation, the hCD47/hSIRPα KI mice were crossed with a mouse humanized for FcγRs, in which all mFcγR genes have been deleted and all hFcγRs are expressed as transgenes (Smith, P. et al. PNAS. 2012. 109, 6181-6186) (FIG. 8A). It was confirmed that the hCD47/hSIRPα/hFcγR mice maintain the human cell-type specific expression profile of FcγRs found in the humanized mice for FcγRs.

First, the inventors hypothesized that the humanized hCD47/hSIRPα/hFcγR mice could recapitulate the toxicity observed with CD47 blocking antibodies in the clinical setting, in particular anemia and thrombocytopenia (Oldenborg, P. A. et al. Science. 2000. 288, 2051-2054). To test this hypothesis, the hCD47 blocking ab 5F9-hIgG4 used in several clinical trials (Advani, R. et al. N Engl J Med. 2018. 379, 1711-1721) was compared with the Fc-optimized variant 5F9-GAALIE. These two antibodies have identical Fab regions, but differ in their Fc: the 5F9-hIgG4 ab is a “weak” engager of FcγRs; in contrast, the 5F9-GAALIE is a hIgG1 Fc format containing three point mutations that enhances binding for all the activating FcγRs and reduced binding to the inhibitory FcγR, thus optimizing the A/I ratio (Weitzenfeld, P. et al. J Clin Invest. 2019. 129, 3952-3962) (FIG. 8B). Systemic administration of these two human antibodies in WT mice did not lead to significant changes in peripheral RBC or platelets in WT mice (FIG. 8C). In contrast, increasing dosing concentration of both 5F9-hIgG4 and 5F9-GAALIE antibodies led to anemia and thrombocytopenia in hCD47/hSIRPα/hFcγR mice. Of note, these effects were transient (lowest values by day 4 to 8) and most of them recovered by day 11. While the 5F9-G4 ab started to decrease the number of RBC and platelets at a high systemic dose (10 mg/Kg) (FIG. 8D), the 5F9-GAALIE ab led to anemia and thrombocytopenia at the lowest systemic dose administered (2.5 mg/Kg), with significantly more anemia, thrombocytopenia, and mortality in mice treated a higher dose (5 mg/Kg and 10 mg/Kg) (FIG. 8E). To understand whether a similar toxicity profile could be seen with murine antibodies, the effects of systemic administration of Fc variants of anti-mCD47 (MIAP301) were compared to C57BL/6 mice at a high dose (10 mg/Kg). The results indicated that the MIAP301-mIgG2a ab led to decreased levels of both RBC and platelets when compared to control or the other Fc variants. However, no mouse mortality was observed, and the decline in RBC and platelet levels induced by this mouse Fc variant was not as severe as the one observed using fully humanized antibodies in the hCD47/hSIRPα/hFcγR mouse model, or those observed in patients receiving these therapies (Advani, R. et al. N Engl J Med. 2018. 379, 1711-1721 and Brierley, C. K. et al. Transfusion. 2019. 59, 2248-2254). Overall, these findings indicate that engagement of activating FcγRs during hCD47 ab blockade also mediates off-tumor on-target toxicity of these therapies, and that hCD47/hSIRPα/hFcγR mice provide a unique and reliable platform for the study of clinically relevant Fc-optimized anti-CD47/SIRPα antibodies in vivo by recapitulating important parameters of biological activity and toxicity observed in the clinical setting.

### Example 8. Fc-Optimized Anti-CD47 Antibodies Promote Superior Antitumor Activity

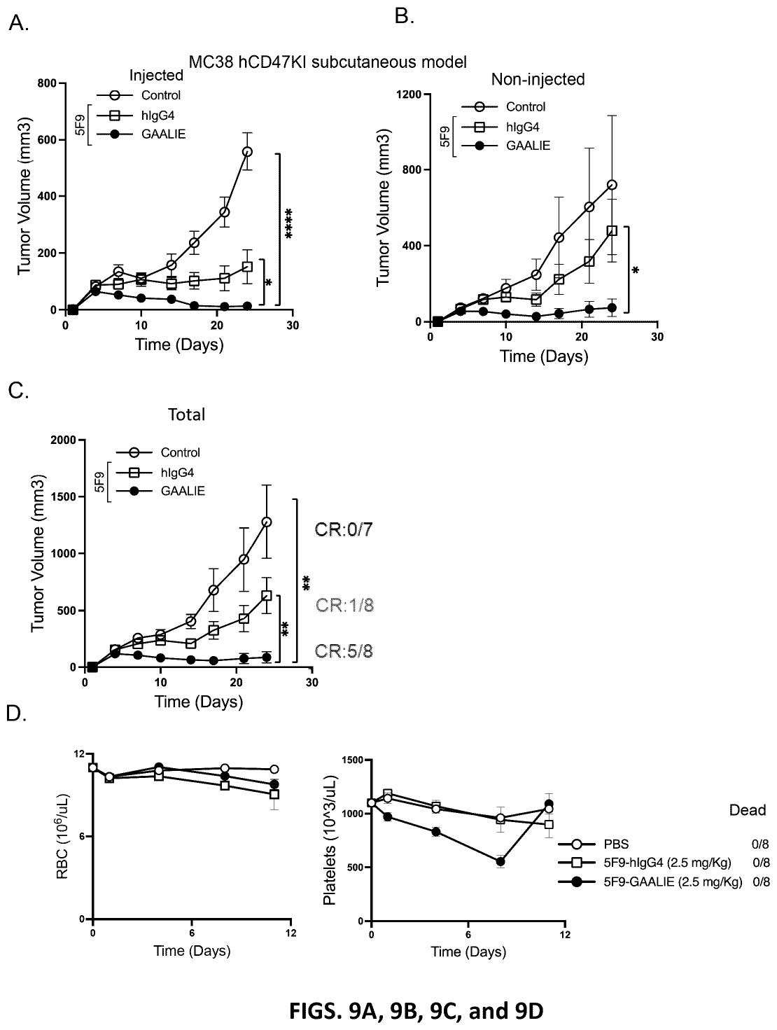

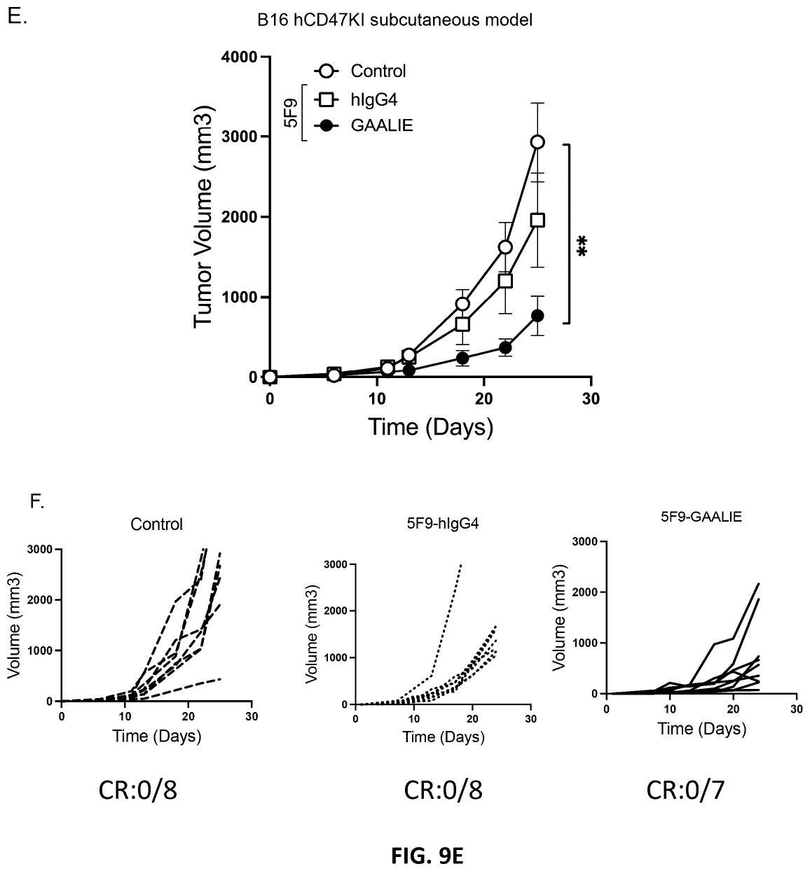

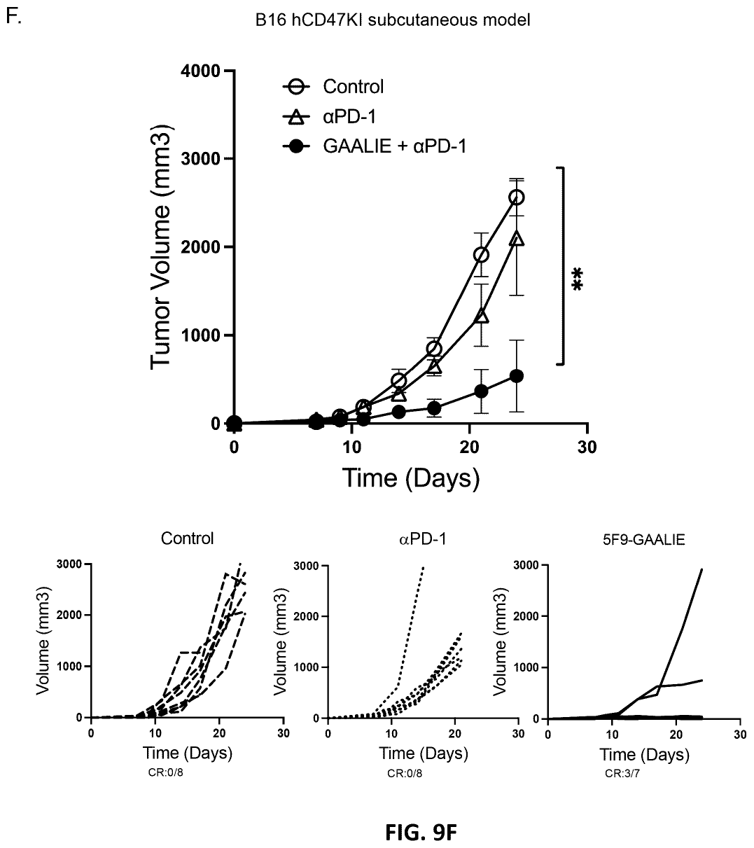

To evaluate the in vitro activity of the two different Fc formats of hCD47 blocking antibodies, the phagocytosis of B16 hCD47 KI or human cancer cells by macrophages generated from species matched mononuclear cells was assessed. The GAALIE 5F9 ab led to significantly more phagocytosis of cancer cells when compared to the hIgG4 format or control (FIGS. 5G-5I). Given the increased Fc-mediated toxicity observed with systemic administration of the 5F9-GAALIE ab, it was next determined whether intratumoral (IT) administration of the lowest dose (2.5 mg/kg) of anti-hCD47 antibodies could help control tumor growth while providing an optimal safety profile. IT administration of the 5F9-GAALIE ab led to a significant reduction in tumor burden when compared to the 5F9-IgG4 ab or control in the MC38 hCD47 KI tumor model (FIG. 9A). Simultaneously, it was evaluated whether this approach could also help control distant (non-injected) tumor growth by inducing an abscopal effect. For this purpose, a bilateral flank MC38 hCD47 KI tumor model was used, which allows monitoring of tumors at both treated and untreated sites. The results indicated that the 5F9-GAALIE ab led to significant control of distant tumor growth when compared to control, ultimately leading to complete responses in 5 out of 8 mice (FIGS. 9B-9C). Of note, this local dose delivery system also resulted in significantly less toxicity (FIG. 9D). To investigate the effects of the 5F9-GAALIE ab in a more aggressive tumor model that is traditionally resistant to single agent immune-based therapies (e.g., anti-PD-1), the B16 hCD47 KI subcutaneous model was used (Dahan, R. et al. Cancer Cell. 2015. 28, 543 and Wei, S. C. et al. Cell. 2017. 170, 1120-1133). The antitumor activity of the two Fc-formats for anti-CD47 ab was compared and the 5F9-GAALIE ab led to a significant decrease in tumor burden as monotherapy when compared to 5F9-hIgG4 or control (FIG. 9E). Furthermore, combination therapy with IT 5F9-GAALIE ab and systemic PD-1 blockade elicited significant improved control of tumor growth when compared to PD-1 blockade administered as monotherapy or control (FIG. 9F). These results indicate that IT dosing of Fc-optimized anti-hCD47 ab promotes effective antitumor activity, leads to abscopal immunity in distant tumor lesions and synergizes with PD-1 blockade while limiting on-target off-tumor toxicity.

### Discussion

The Fc region of antibodies blocking CD47 has been postulated as a potential modulator of their therapeutic activity (Bouwstra, R. et al. Clin Transl Med. 2022. 12, e943; Zhao, X. W. et al. PNAS. 2012. 109, E2843; author reply E2844-2845; and Maute, R. et al. Immunooncol Technol. 2022. 13, 100070). However, efforts to address this question have been limited to suboptimal preclinical studies, limiting their translational relevance for human therapeutics. The above-described study overcame these limitations by elucidating the in vivo contributions of the Fc region of anti-CD47 antibodies using a novel syngeneic immunocompetent murine model that accurately reflects the affinities and cellular expression of hCD47-hSIRPα and hIgG-hFcγRs axes. As a proof of concept, multiple species matched systems were used to demonstrate that anti-CD47 antibodies displayed enhanced in vivo antitumor activity only when the ab Fc was optimized to engage activating FcγRs. This effect depends on both macrophages and antigen specific T cells and leads to long-lived systemic immunity. Using this unique platform, the inventors demonstrated enhanced in vivo antitumor activity, abscopal antitumor effect, and minimal on-target toxicity induced by local administration of the fully human Fc-optimized anti-CD47 ab 5F9-GAALIE as monotherapy or in combination with PD-1 blockade. These data support the importance of Fc optimization in the development of effective anti-CD47 therapies and provide a reliable platform to optimize the therapeutic activity of this promising immunotherapy.

These studies demonstrate that anti-CD47 antibodies require two mechanisms of action for optimal in vivo activity: 1) Fab-mediated CD47/SIRPα-blocking signal; and 2) Fc-mediated engagement of activating—but not inhibitory—FcγRs. This dual mechanism of action has been considered a critical factor for the activity of other immunomodulatory antibodies. This is the case of antibodies blocking CTLA-4, CCR8, OX-40, 401BB or PD-L1, in which engagement of activating FcγRs seems to augment their therapeutic activity (Cohen Saban et al. Sci Immunol. 2023. 8, eadd8005; Simpson, T. R. et al. J Exp Med. 2013. 210, 1695-1710; Arce Vargas, F. et al. Cancer Cell. 2018. 33, 649-663; Bulliard, Y. et al. Immunol Cell Biol. 2014. 92, 475-480; Buchan, S. L. et al. Immunity. 2018. 49, 958-970; and Knorr, D. et al. bioRxiv. 2023. 10.1101/2023.01.19.522856). In contrast, selective engagement of the inhibitory FcγRIIB enhances the activity of agonistic antibodies targeting CD40 (Li, F. et al. Science. 2011. 333, 1030-1034 and White, A. L. et al. J Immunol. 2011. 187, 1754-1763). In certain cases, engagement of FcγRs could also compromise the activity of these therapies, which is the case of anti-PD-1 antibodies (Dahan, R. et al. Cancer Cell. 2015. 28, 543). Furthermore, the optimization of the Fc region of the fully humanized anti-CD47 ab to selectively engage activating FcγRs significantly augmented the therapeutic activity of these agents in a system that closely recapitulate both human CD47-SIRPα and IgG-FcγR signaling. This is important, given the extensive differences on the pattern of cellular expression and distribution between the mouse and human FcγRs of macrophages and other immune cells in the TME (Bournazos, S. Curr Top Microbiol Immunol. 2019. 423, 1-11). These differences further underscores the advantage of using fully humanized Fc-FcγR systems to better estimate the activity of these therapies in humans (Bournazos, S. et al. Curr Top Microbiol Immunol. 2014. 382, 237-248). Altogether, these studies point to the clinical development and evaluation of second-generation CD47 blocking antibodies with enhanced Fc effector function, which could improve their therapeutic activity and overcome some of the limited clinical benefit observed in early-phase clinical trials.

The improved in vivo activity of anti-CD47 antibodies Fc-enhanced for activating FcγRs is characterized by modulation of several immune cell populations in the TME. Notably, a significant increase was found in macrophages in the TME of treated mice, which likely contribute to their antitumor activity. The central role of macrophages on the in vivo Fc-mediated activity of anti-CD47 antibodies is consistent with the above findings and others indicating that Fc-FcγR interactions are required to induce significant phagocytosis of cancer cells by human macrophages during treatment with CD47 therapies (Jain, S. et al. 2019. 134, 1430-1440 and Metayer, L. E. et al. Oncotarget. 2017. 8, 60892-60903). This suggests that in order to achieve effective antitumor activity, anti-CD47 antibodies must not only disrupt CD47-SIRPα interactions in macrophages, but also opsonize the tumor cells through antibody-dependent cellular phagocytosis. Furthermore, downstream interactions between SIRPα and FcγR signaling seem to be critical for the effector activity of these cells, with evidence indicating the ratio of activating IgG to inhibitory CD47 blockade dictates macrophage phagocytic activity by increasing phosphorylation of immunoreceptor tyrosine-based activation motifs on FcγRs (Suter, E. C. et al. Cell Rep. 2021. 36, 109587).

In addition to the increased number of macrophages in the TME, a decrease in the frequency of T_{regs}, and an increase in the number of activated CD8⁺ T cells and CD8⁺ antigen-specific T cells was observed. T_{regs} are a specialized sub-population of T cells that diminish effective antitumor immune responses and are an attractive target for cancer immunotherapy (Togashi, Y. et al. Nat Rev Clin Oncol. 2019. 16, 356-371). Of note, T_{regs} express high levels of CD47 when compared to other CD4⁺ or CD8⁺ T cells, a finding that has been observed in CTLA-4⁺ T_{regs} obtained from human tumor samples (Zhang, A. et al. Sci Transl Med. 2021. 13.10.1126/scitranslmed.abg8693). While reduced differentiation or proliferation of T_{regs} could be induced by “reprogrammed” macrophages after Fc-mediated phagocytosis of cancer cells (Tseng, D. et al. PNAS. 2013. 110, 11103-11108), the above data suggest that Fc-optimized anti-CD47 antibodies promote Treg depletion likely contributing to a less immunosuppressive TME. Nevertheless, these findings suggest that in addition to modulation of myeloid effector activity, Fc-optimized anti-CD47 antibodies also promote systemic and long-lasting adaptive immune responses by downregulating immunosuppressive T cell populations and enabling effective tumor antigen-specific cytotoxic T cell responses. The alterations in the frequency of these T cell populations in the TME may also explain the abscopal effects observed after local administration of the CD47 ab 5F9-GAALIE, as well the long-term immune memory in the tumor rechallenge experiments.

On-target toxicity resulting from phagocytosis of normal CD47-expressing cells (i.e., RBC and platelets) during treatment with anti-CD47 antibodies remains a concern for the clinical implementation of these therapies. The results using mouse and human anti-CD47 antibodies in WT C57BL/6 mice reaffirm the suboptimal nature of this model to recapitulate the toxicity observed in humans, given the lack of cross-reactivity between most human CD47 blocking antibodies and their murine counterparts (Huang, Y. et al. J Thorac Dis. 2017. 9, E168-E174). Furthermore, these systems may overestimate the efficacy of these therapies as they failed to account for the large CD47 antigenic sink from normal cells. Nonhuman primates are a better system to assess toxicity (Liu, J. et al. PLOS One. 2015. 10, e0137345; Meng, Z. et al. Blood. 2019. 134. 10.1182/blood-2019-122793; and Puro, R. J. et al. Molecular Cancer Therapeutics. 2020. 19, 835-846), however their use for simultaneous assessment of antitumor activity or alternative delivery or dosing systems are limited. The humanized model described herein overcomes these limitations and provides a system for simultaneous assessment of on-target off-tumor toxicity and antitumor activity. The results indicate that the Fc region of CD47 antibodies contributes to such toxicities. These results have significant implications on the development of novel CD47 blocking agents that balance the anti-cancer Fc-mediated effects with the potential hazardous off-tumor side effects. To overcome this limitation, IT delivery of the 5F9-GAALIE ab was tested, which maximizes durable control of local and distant tumor growth while minimizing on-target toxicity. Finally, it was observed that combination with therapies, such as PD-1 blockade, can further enhance the systemic long-term immunity and is an attractive potential avenue to explore in patients.

The CD47-SIRPα axis is a promising target in cancer immunotherapy with a rapid developing field in recent years. By elucidating the in vivo contributions of FcγR signaling in the antitumor activity and toxicity of fully humanized anti-CD47 antibodies, these findings address a critical question in the field that will inform the rational development of Fc-optimized anti-CD47 antibodies.

Sequence Listing

UpCD47C (SEQ ID NO: 1):
TTGCATCGTCCGTAATGTGGAGG
DnCD47D (SEQ ID NO: 2):
ATAGAGCTGAAAAACCGCACGGG
UpSIRPαA (SEQ ID NO: 3):
AAATCAGTGTCTGTTGCTGCTGG
UpSIRPαE (SEQ ID NO: 4)
GGAACAGAGGTCTATGTACTCGG
CRISPR sgRNA (SEQ ID NO: 5):
U*C*U*GCAUAUAUGAUUAUCUU
CRISPR sgRNA (SEQ ID NO: 6):
A*C*C*UUGCAGAAGUCACUAG
Human CD47 (SEQ ID NO: 7):
MWPLVAALLLGSACCGSAQLLFNKTKSVEFTFCNDTVVIPCFVTNMEAQNTTEVYVKW
KFKGRDIYTFDGALNKSTVPTDFSSAKIEVSQLLKGDASLKMDKSDAVSHTGNYTCEVT
ELTREGETIIELKYRVVSWFSPNENILIVIFPIFAILLFWGQFGIKTLKYRSGGMDEKTIALL
VAGLVITVIVIVGAILFVPGEYSLKNATGLGLIVTSTGILILLHYYVFSTAIGLTSFVIAILVI
QVIAYILAVVGLSLCIAACIPMHGPLLISGLSILALAQLLGLVYMKFVASNQKTIQPPRKA
VEEPLNAFKESKGMMNDE
hIgG1 (GAALIE: G236A/A330L/I332E) (SEQ ID NO: 8):
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLAG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPLPEEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
mIgG1 (D265A) (SEQ ID NO: 9):
TKGPSVYPLAPGSAAQTNSMVTLGCLVKGYFPEPVTVTWNSGSLSSGVHTFPAVLQSDL
YTLSSSVTVPSSTWPSQTVTCNVAHPASSTKVDKKIVPRDCGCKPCICTVPEVSSVFIFPP
KPKDVLTITLTPKVTCVVVAISKDDPEVQFSWFVDDVEVHTAQTKPREEQINSTFRSVSE
LPIMHQDWLNGKEFKCRVNSAAFPAPIEKTISKTKGRPKAPQVYTIPPPKEQMAKDKVS
LTCMITNFFPEDITVEWQWNGQPAENYKNTQPIMDTDGSYFVYSKLNVQKSNWEAGNT
FTCSVLHEGLHNHHTEKSLSHSPGK
mIgG2a (SEQ ID NO: 10):
TTAPSVYPLAPVCGGTTGSSVTLGCLVKGYFPEPVTLTWNSGSLSSGVHTFPALLQSGLY
TLSSSVTVTSNTWPSQTITCNVAHPASSTKVDKKIEPRVPITQNPCPPLKECPPCAAPDLL
GGPSVFIFPPKIKDVLMISLSPMVTCVVVDVSEDDPDVQISWFVNNVEVHTAQTQTHRE
DYNSTLRVVSALPIQHQDWMSGKEFKCKVNNRALPSPIEKTISKPRGPVRAPQVYVLPPP
AEEMTKKEFSLTCMITGFLPAEIAVDWTSNGRTEQNYKNTATVLDSDGSYFMYSKLRV
QKSTWERGSLFACSVVHEGLHNHLTTKTISRSLGK
5F9 VH (SEQ ID NO: 11):
QVQLVQSGAEVKKPGASVKVSCKASGYTFTNYNMHWVRQAPGQRLEWMGTIYPGND
DTSYNQKFKDRVTITADTSASTAYMELSSLRSEDTAVYYCARGGYRAMDYWGQGTLV
TVSS
5F9 VL (SEQ ID NO: 12):
DIVMTQSPLSLPVTPGEPASISCRSSQSIVYSNGNTYLGWYLQKPGQSPQLLIYKVSNRFS
GVPDRFSGSGSGTDFTLKISRVEAEDVGVYYCFQGSHVPYTFGQGTKLEIK
Wild-type IgG1 (SEQ ID NO: 13):
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
HCDR1 5F9 (SEQ ID NO: 14):
NYNMH
HCDR2 5F9 (SEQ ID NO: 15):
TIYPGNDDTSYNQKFKD
HCDR3 5F9 (SEQ ID NO: 16):
GGYRAMDY
LCDR1 5F9 (SEQ ID NO: 17):
CRSSQSIV
LCDR2 5F9 (SEQ ID NO: 18):
KVSNRFS
LCDR3 F59 (SEQ ID NO: 19):
FQGSHVPYT
HC 5F9 (SEQ ID NO: 20):
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLAG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPLPEEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
LC 5F9 (SEQ ID NO: 21):
DIVMTQSPLSLPVTPGEPASISCRSSQSIVYSNGNTYLGWYLQKPGQSPQLLIYKVSNRFS
GVPDRFSGSGSGTDFTLKISRVEAEDVGVYYCFQGSHVPYTFGQGTKLEIKVDAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
HCDR1 MIAP301 (SEQ ID NO: 22): GYSFTDYW
HCDR2 MIAP301 (SEQ ID NO: 23): IYPSNTVT
HCDR3 MIAP301 (SEQ ID NO: 24): TRLGNSGY
LCDR1 MIAP301 (SEQ ID NO: 25): ESVSSH
LCDR2 MIAP301 (SEQ ID NO: 26): YKASN
LCDR3 MIAP301 (SEQ ID NO: 27): QQGWNGPFT
VH MIAP301 (SEQ ID NO: 28):
QVKLLQSGAALVKPGASVKMSCQASGYSFTDYWVTWVKQSHGQSLEWIGEIYPSNTVT
NFNDNFKGKATLTVDKSTSTAYMELSRLTSEDSAIYYCTRLGNSGYRVGWFLYWGQGT
LVTVISAS
VL MIAP301 (SEQ ID NO: 29):
SDTVLTQSPALAVSPGERVTISCKASESVSSHMHWYQQKPGQQPKLLIYKASNLASGVP
ARFSGSGSGTDFTLTIDPVEADDTATYFCQQGWNGPFTFGAGTRLELKRAVD
HC MIAP301 (SEQ ID NO: 30):
QVKLLQSGAALVKPGASVKMSCQASGYSFTDYWVTWVKQSHGQSLEWIGEIYPSNTVT
NFNDNFKGKATLTVDKSTSTAYMELSRLTSEDSAIYYCTRLGNSGYRVGWFLYWGQGT
LVTVISASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCPA
PELLAGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPLPEEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
LC MIAP301 (SEQ ID NO: 31):
SDTVLTQSPALAVSPGERVTISCKASESVSSHMHWYQQKPGQQPKLLIYKASNLASGVP
ARFSGSGSGTDFTLTIDPVEADDTATYFCQQGWNGPFTFGAGTRLELKRAVDAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
HCDR1 5F9 (SEQ ID NO: 32): AATTACAATATGCAC
HCDR2 5F9 (SEQ ID NO: 33):
ACTATTTATCCAGGAAATGATGATACTTCCTACAATCAGAAGTTCAAAGAC
HCDR3 5F9 (SEQ ID NO: 34):
GGGGGTTATCGTGCTATGGACTAC
LCDR1 5F9 (SEQ ID NO: 35):
TGCAGATCTAGTCAGAGCATTGTA
LCDR2 5F9 (SEQ ID NO: 36):
AAAGTTTCCAACCGATTTTCT
LCDR3 5F9 (SEQ ID NO: 37):
TTTCAAGGTTCACATGTTCCGTACACG
VH 5F9 (SEQ ID NO: 38):
CAGGTGCAGCTGGTGCAGTCTGGGGCTGAGGTGAAGAAGCCTGGGGCCTCAGTGAA
GGTTTCCTGCAAGGCATCTGGATACACCTTCACCAATTACAATATGCACTGGGTGCG
ACAGGCCCCTGGACAACGGCTTGAGTGGATGGGAACTATTTATCCAGGAAATGATG
ATACTTCCTACAATCAGAAGTTCAAAGACAGGGTCACAATCACTGCAGACACATCC
GCGAGCACAGCCTACATGGAGCTGAGCAGCCTGAGATCTGAGGACACGGCCGTGTA
TTACTGTGCTAGAGGGGGTTATCGTGCTATGGACTACTGGGGTCAAGGAACCCTGGT
CACCGTCTCCTCA
VL 5F9 (SEQ ID NO: 39):
GATATCGTGATGACTCAGTCTCCACTCTCCCTGCCCGTCACCCCAGGAGAGCCGGCC
TCCATCTCCTGCAGATCTAGTCAGAGCATTGTATATAGTAATGGAAACACCTATTTA
GGATGGTACCTGCAGAAGCCAGGCCAATCTCCACAGCTCCTGATCTACAAAGTTTCC
AACCGATTTTCTGGGGTCCCAGACAGATTCAGCGGCAGTGGGTCAGGCACTGATTTC
ACACTGAAAATCAGCAGGGTGGAGGCTGAGGATGTTGGGGTTTATTACTGCTTTCAA
GGTTCACATGTTCCGTACACGTTCGGACAGGGGACCAAGCTGGAAATAAAA
HC 5F9 (SEQ ID NO: 40):
CAGGTGCAGCTGGTGCAGTCTGGGGCTGAGGTGAAGAAGCCTGGGGCCTCAGTGAA
GGTTTCCTGCAAGGCATCTGGATACACCTTCACCAATTACAATATGCACTGGGTGCG
ACAGGCCCCTGGACAACGGCTTGAGTGGATGGGAACTATTTATCCAGGAAATGATG
ATACTTCCTACAATCAGAAGTTCAAAGACAGGGTCACAATCACTGCAGACACATCC
GCGAGCACAGCCTACATGGAGCTGAGCAGCCTGAGATCTGAGGACACGGCCGTGTA
TTACTGTGCTAGAGGGGGTTATCGTGCTATGGACTACTGGGGTCAAGGAACCCTGGT
CACCGTCTCCTCAGCGTCGACCAAGGGCCCATCGGTCTTCCCCCTGGCACCCTCCTC
CAAGAGCACCTCTGGGGGCACAGCGGCCCTGGGCTGCCTGGTCAAGGACTACTTCC
CCGAACCTGTGACGGTCTCGTGGAACTCAGGCGCCCTGACCAGCGGCGTGCACACC
TTCCCGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGCCCAG
CAACACCAAGGTGGACAAGAGAGTTGAGCCCAAATCTTGTGACAAAACTCACACAT
GCCCACCGTGCCCAGCACCTGAACTCCTGGCGGGACCGTCAGTCTTCCTCTTCCCCC
CAAAACCCAAGGACACCCTCATGATCTCCCGGACCCCTGAGGTCACATGCGTGGTG
GTGGACGTGAGCCACGAAGACCCTGAGGTCAAGTTCAACTGGTACGTGGACGGCGT
GGAGGTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAGGAGTA
CAAGTGCAAGGTCTCCAACAAAGCCCTCCCACTCCCCGAGGAGAAAACCATCTCCA
AAGCCAAAGGGCAGCCCCGAGAACCACAGGTGTACACCCTGCCCCCATCCCGGGAG
GAGATGACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACAAGACC
ACGCCTCCCGTGCTGGACTCCGACGGCTCCTTCTTCCTCTATAGCAAGCTCACCGTG
GACAAGAGCAGGTGGCAGCAGGGGAACGTCTTCTCATGCTCCGTGATGCATGAGGC
TCTGCACAACCACTACACGCAGAAGAGCCTCTCCCTGTCCCCGGGTAAA
LC 5F9 (SEQ ID NO: 41):
GATATCGTGATGACTCAGTCTCCACTCTCCCTGCCCGTCACCCCAGGAGAGCCGGCC
TCCATCTCCTGCAGATCTAGTCAGAGCATTGTATATAGTAATGGAAACACCTATTTA
GGATGGTACCTGCAGAAGCCAGGCCAATCTCCACAGCTCCTGATCTACAAAGTTTCC
AACCGATTTTCTGGGGTCCCAGACAGATTCAGCGGCAGTGGGTCAGGCACTGATTTC
ACACTGAAAATCAGCAGGGTGGAGGCTGAGGATGTTGGGGTTTATTACTGCTTTCAA
GGTTCACATGTTCCGTACACGTTCGGACAGGGGACCAAGCTGGAAATAAAAGTCGA
CGCACCATCTGTCTTCATCTTCCCGCCATCTGATGAGCAGTTGAAATCTGGAACTGC
CTCTGTTGTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGCAGGACA
GCAAGGACAGCACCTACAGCCTCAGCAGCACCCTGACGCTGAGCAAAGCAGACTAC
GAGAAACACAAAGTCTACGCCTGCGAAGTCACCCATCAGGGCCTGAGCTCGCCCGT
CACAAAGAGCTTCAACAGGGGAGAGTGT
HCDR1 MIAP301 (SEQ ID NO: 42): GGTTATTCATTCACTGACTACTGG
HCDR2 MIAP301 (SEQ ID NO: 43): ATTTATCCTAGCAATACTGTTACT
HCDR3 MIAP301 (SEQ ID NO: 44): ACAAGATTGGGGAATTCGGGGTAC
LCDR1 MIAP301 (SEQ ID NO: 45): GAAAGTGTCAGTTCACAT
LCDR2 MIAP301 (SEQ ID NO: 46): TATAAAGCATCAAAC
LCDR3 MIAP301 (SEQ ID NO: 47): CAGCAGGGTTGGAATGGTCCGTTCACG
VH MIAP301 (SEQ ID NO: 48):
CAGGTCAAGCTGCTGCAGTCTGGGGCTGCACTGGTGAAGCCTGGAGCCTCTGTGAA
GATGTCTTGCCAAGCCTCTGGTTATTCATTCACTGACTACTGGGTGACCTGGGTGAA
GCAGAGTCATGGACAGAGCCTTGAGTGGATTGGGGAAATTTATCCTAGCAATACTGT
TACTAACTTCAATGATAACTTCAAGGGCAAGGCCACATTGACTGTAGACAAATCCAC
CAGCACAGCCTATATGGAGCTCAGCAGATTGACATCTGAGGACTCTGCAATCTATTA
CTGTACAAGATTGGGGAATTCGGGGTACAGAGTTGGTTGGTTTCTTTACTGGGGCCA
AGGCACTCTGGTCACTGTCATTTCAGCGTCG
VL MIAP301 (SEQ ID NO: 49):
TCCGACACTGTGCTGACCCAGTCTCCTGCTTTGGCTGTGTCTCCTGGAGAGAGGGTT
ACCATCTCCTGTAAGGCCAGTGAAAGTGTCAGTTCACATATGCACTGGTACCAACAG
AAACCAGGACAGCAACCCAAACTCCTCATCTATAAAGCATCAAACCTAGCATCTGG
GGTCCCTGCCAGGTTCAGTGGCAGTGGGTCTGGGACAGACTTCACCCTCACCATTGA
TCCTGTGGAGGCTGATGACACTGCAACCTACTTCTGTCAGCAGGGTTGGAATGGTCC
GTTCACGTTTGGAGCTGGGACCAGGCTGGAACTGAAACGGGCTGTCGAC
HC MIAP301 (SEQ ID NO: 50):
CAGGTCAAGCTGCTGCAGTCTGGGGCTGCACTGGTGAAGCCTGGAGCCTCTGTGAA
GATGTCTTGCCAAGCCTCTGGTTATTCATTCACTGACTACTGGGTGACCTGGGTGAA
GCAGAGTCATGGACAGAGCCTTGAGTGGATTGGGGAAATTTATCCTAGCAATACTGT
TACTAACTTCAATGATAACTTCAAGGGCAAGGCCACATTGACTGTAGACAAATCCAC
CAGCACAGCCTATATGGAGCTCAGCAGATTGACATCTGAGGACTCTGCAATCTATTA
CTGTACAAGATTGGGGAATTCGGGGTACAGAGTTGGTTGGTTTCTTTACTGGGGCCA
AGGCACTCTGGTCACTGTCATTTCAGCGTCGACCAAGGGCCCATCGGTCTTCCCCCT
GGCACCCTCCTCCAAGAGCACCTCTGGGGGCACAGCGGCCCTGGGCTGCCTGGTCA
AGGACTACTTCCCCGAACCTGTGACGGTCTCGTGGAACTCAGGCGCCCTGACCAGCG
GCGTGCACACCTTCCCGGCTGTCCTACAGTCCTCAGGACTCTACTCCCTCAGCAGCG
TGGTGACCGTGCCCTCCAGCAGCTTGGGCACCCAGACCTACATCTGCAACGTGAATC
ACAAGCCCAGCAACACCAAGGTGGACAAGAGAGTTGAGCCCAAATCTTGTGACAAA
ACTCACACATGCCCACCGTGCCCAGCACCTGAACTCCTGGCGGGACCGTCAGTCTTC
CTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTACAAC
AGCACGTACCGTGTGGTCAGCGTCCTCACCGTCCTGCACCAGGACTGGCTGAATGGC
AAGGAGTACAAGTGCAAGGTCTCCAACAAAGCCCTCCCACTCCCCGAGGAGAAAAC
CATCTCCAAAGCCAAAGGGCAGCCCCGAGAACCACAGGTGTACACCCTGCCCCCAT
CCCGGGAGGAGATGACCAAGAACCAGGTCAGCCTGACCTGCCTGGTCAAAGGCTTC
TATCCCAGCGACATCGCCGTGGAGTGGGAGAGCAATGGGCAGCCGGAGAACAACTA
CAAGACCACGCCTCCCGTGCTGGACTCCGACGGCTCCTTCTTCCTCTATAGCAAGCT
CACCGTGGACAAGAGCAGGTGGCAGCAGGGGAACGTCTTCTCATGCTCCGTGATGC
ATGAGGCTCTGCACAACCACTACACGCAGAAGAGCCTCTCCCTGTCCCCGGGTAAA
LC MIAP301 (SEQ ID NO: 51):
TCCGACACTGTGCTGACCCAGTCTCCTGCTTTGGCTGTGTCTCCTGGAGAGAGGGTT
ACCATCTCCTGTAAGGCCAGTGAAAGTGTCAGTTCACATATGCACTGGTACCAACAG
AAACCAGGACAGCAACCCAAACTCCTCATCTATAAAGCATCAAACCTAGCATCTGG
GGTCCCTGCCAGGTTCAGTGGCAGTGGGTCTGGGACAGACTTCACCCTCACCATTGA
TCCTGTGGAGGCTGATGACACTGCAACCTACTTCTGTCAGCAGGGTTGGAATGGTCC
GTTCACGTTTGGAGCTGGGACCAGGCTGGAACTGAAACGGGCTGTCGACGCACCAT
CTGTCTTCATCTTCCCGCCATCTGATGAGCAGTTGAAATCTGGAACTGCCTCTGTTGT
GTGCCTGCTGAATAACTTCTATCCCAGAGAGGCCAAAGTACAGTGGAAGGTGGATA
ACGCCCTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAGAAACA
CAAAGTCTACGCCTGCGAAGTCACCCATCAGGGCCTGAGCTCGCCCGTCACAAAGA
GCTTCAACAGGGGAGAGTGT
Note:
PAMs are in bold. Bold and underlined indicates an amino acid substitution.

What is claimed is:

1. An isolated antibody or antigen-binding portion thereof that specifically binds to human CD47, wherein the antitumor activity of the antibody or antigen-binding portion thereof comprises an Fc region modified to enhance the antitumor activity of the antibody or antigen-binding portion thereof, and wherein the Fc region comprises at least one mutation that is G236A/A330L/1332E with respect to SEQ ID NO: 13.

2. The antibody or antigen-binding portion thereof of claim 1, wherein the Fc region comprises a hIgG1 having G236A/A330L/1332E mutations as set forth in SEQ ID NO: 8.

3. The antibody or antigen-binding portion thereof of claim 1, wherein the antibody or antigen-binding portion thereof comprises 5F9 or MIAP301.

4. The antibody or antigen-binding portion thereof of claim 1, wherein the antibody or antigen-binding portion thereof comprises a heavy chain variable region having the amino acid sequence of SEQ ID NO: 11 and a light chain variable region having the amino acid sequence of SEQ ID NO: 12.

5. The antibody or antigen-binding portion thereof of claim 1, wherein the antibody or antigen-binding portion thereof increases phagocytosis of cancer cells.

6. The antibody or antigen-binding portion thereof of claim 1, wherein the antibody or antigen-binding portion thereof blocks the interaction of CD47 with hSIRPα.

7. The antibody or antigen-binding portion thereof of claim 1, wherein the antibody or antigen-binding portion thereof is humanized.

8. A nucleic acid comprising a sequence encoding the antibody or antigen-binding portion thereof of claim 1.

9. An expression vector comprising the nucleic acid of claim 8.

10. A host cell comprising the nucleic acid of claim 8.

11. A pharmaceutical formulation comprising the antibody or antigen-binding portion thereof of claim 1 and optionally a pharmaceutically acceptable carrier.

12. A method of treating cancer, comprising administering to a subject in need thereof a therapeutically effective amount of the antibody or antigen-binding portion thereof claim 1 or a nucleic acid encoding said antibody or antigen-binding portion thereof.

13. The method of claim 12, wherein the cancer is a CD47 expressing cancer comprising colorectal cancer, lymphoma, lung cancer, melanoma, breast cancer, ovarian cancer, endometrial cancer, cervical cancer, bladder cancer, prostate cancer, kidney cancer, glioma or glioblastoma, gastric cancer, esophageal carcinoma, osteosarcoma, or head and neck cancer.

14. The method of claim 12, further comprising administering a second therapeutic agent.

15. The method of claim 13, wherein the second therapeutic agent is a checkpoint inhibitor or a tumor antigen inhibitor.

16. A mouse model for evaluating an anti-CD47 antibody or antigen-binding portion thereof, wherein the mouse comprises a humanized CD47, a humanized SIRPα, and humanized FcγRs.

17. The mouse model of claim 16, wherein the mouse recapitulates the toxicity observed with a blocking anti-CD47 antibody or antigen-binding portion thereof in a clinical setting.

18. The mouse model of claim 16, wherein the toxicity comprises anemia or thrombocytopenia.

19. A method of generating the mouse model of claim 16, comprising administering to the mouse a nucleic acid molecule comprising or encoding one or more CRISPR sgRNAs having a nucleotide sequence of SEQ ID NO: 1, SEQ ID NO: 2, SEQ ID NO: 3, or SEQ ID NO: 4.

20. The method of claim 19, wherein the mouse is backcrossed with a hFcγR mouse to generate hCD47/hSIRPα/hFcγR mice.

21. A method of boosting systemic antitumor immunity with minimal toxicity, comprising intratumorally administering to a subject in need thereof a therapeutically effective amount of an isolated antibody or antigen-binding portion thereof that specifically binds to human CD47, wherein the antibody or antigen-binding portion thereof comprises an Fc region modified to enhance the antitumor activity of the antibody or antigen-binding portion thereof, and wherein the Fc region comprises at least one mutation that is G236A/A330L/1332E with respect to SEQ ID NO: 13.

22. The method of claim 21, wherein the antibody comprises a hIgG1 having G236A/A330L/1332E mutations as set forth in SEQ ID NO: 8.

23. The method of claim 21, wherein the antibody or antigen-binding portion thereof comprises 5F9 or MIAP301.

24. The method of claim 21, wherein the antibody or antigen-binding portion thereof comprises a heavy chain variable region having the amino acid sequence of SEQ ID NO: 11 and a light chain variable region having the amino acid sequence of SEQ ID NO: 12.

25. The method of claim 21, wherein the antibody or antigen-binding portion thereof is humanized.


SEQUENCE LISTING (51 sequences)

SEQ ID NO: 1 - DNA, 23 bp, Synthetic construct
  source 1..23: mol_type = other DNA
ttgcatcgtccgtaatgtggagg

SEQ ID NO: 2 - DNA, 23 bp, Synthetic construct
  source 1..23: mol_type = other DNA
atagagctgaaaaaccgcacggg

SEQ ID NO: 3 - DNA, 23 bp, Synthetic construct
  source 1..23: mol_type = other DNA
aaatcagtgtctgttgctgctgg

SEQ ID NO: 4 - DNA, 23 bp, Synthetic construct
  source 1..23: mol_type = other DNA
ggaacagaggtctatgtactcgg

SEQ ID NO: 5 - DNA, 20 bp, Synthetic construct
  source 1..20: mol_type = other DNA
tctgcatatatgattatctt

SEQ ID NO: 6 - DNA, 19 bp, Synthetic construct
  source 1..19: mol_type = other DNA
accttgcagaagtcactag

SEQ ID NO: 7 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 8 - protein, 330 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 322 aa, Synthetic construct
TKGPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 10 - protein, 333 aa, Mus musculus
TTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 11 - protein, 117 aa, Synthetic construct
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 12 - protein, 112 aa, Synthetic construct
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 13 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 5 aa, Synthetic construct
NYNMH

SEQ ID NO: 15 - protein, 17 aa, Synthetic construct
TIYPGNDDTSYNQKFKD

SEQ ID NO: 16 - protein, 8 aa, Synthetic construct
GGYRAMDY

SEQ ID NO: 17 - protein, 8 aa, Synthetic construct
CRSSQSIV

SEQ ID NO: 18 - protein, 7 aa, Synthetic construct
KVSNRFS

SEQ ID NO: 19 - protein, 9 aa, Synthetic construct
FQGSHVPYT

SEQ ID NO: 20 - protein, 330 aa, Synthetic construct; identical to SEQ ID NO: 8

SEQ ID NO: 21 - protein, 217 aa, Synthetic construct
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKVDAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 8 aa, Synthetic construct
GYSFTDYW

SEQ ID NO: 23 - protein, 8 aa, Synthetic construct
IYPSNTVT

SEQ ID NO: 24 - protein, 8 aa, Synthetic construct
TRLGNSGY

SEQ ID NO: 25 - protein, 6 aa, Synthetic construct
ESVSSH

SEQ ID NO: 26 - protein, 5 aa, Synthetic construct
YKASN

SEQ ID NO: 27 - protein, 9 aa, Synthetic construct
QQGWNGPFT

SEQ ID NO: 28 - protein, 124 aa, Synthetic construct
QVKLLQSGAALVKPGASVKMSCQASGYSFTDYWVTWVKQSHGQSLEWIGEIYPSNTVTNFNDNFKGKATLTVDKSTSTAYMELSRLTSEDSAIYYCTRLGNSGYRVGWFLYWGQGTLVTVISAS

SEQ ID NO: 29 - protein, 111 aa, Synthetic construct
SDTVLTQSPALAVSPGERVTISCKASESVSSHMHWYQQKPGQQPKLLIYKASNLASGVPARFSGSGSGTDFTLTIDPVEADDTATYFCQQGWNGPFTFGAGTRLELKRAVD

SEQ ID NO: 30 - protein, 452 aa, Synthetic construct
QVKLLQSGAALVKPGASVKMSCQASGYSFTDYWVTWVKQSHGQSLEWIGEIYPSNTVTNFNDNFKGKATLTVDKSTSTAYMELSRLTSEDSAIYYCTRLGNSGYRVGWFLYWGQGTLVTVISASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 214 aa, Synthetic construct
SDTVLTQSPALAVSPGERVTISCKASESVSSHMHWYQQKPGQQPKLLIYKASNLASGVPARFSGSGSGTDFTLTIDPVEADDTATYFCQQGWNGPFTFGAGTRLELKRAVDAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - DNA, 15 bp, Synthetic construct
  source 1..15: mol_type = other DNA
aattacaatatgcac

SEQ ID NO: 33 - DNA, 51 bp, Synthetic construct
  source 1..51: mol_type = other DNA
actatttatccaggaaatgatgatacttcctacaatcagaagttcaaagac

SEQ ID NO: 34 - DNA, 24 bp, Synthetic construct
  source 1..24: mol_type = other DNA
gggggttatcgtgctatggactac

SEQ ID NO: 35 - DNA, 24 bp, Synthetic construct
  source 1..24: mol_type = other DNA
tgcagatctagtcagagcattgta

SEQ ID NO: 36 - DNA, 21 bp, Synthetic construct
  source 1..21: mol_type = other DNA
aaagtttccaaccgattttct

SEQ ID NO: 37 - DNA, 27 bp, Synthetic construct
  source 1..27: mol_type = other DNA
tttcaaggttcacatgttccgtacacg

SEQ ID NO: 38 - DNA, 351 bp, Synthetic construct
  source 1..351: mol_type = other DNA
caggtgcagctggtgcagtctggggctgaggtgaagaagcctggggcctcagtgaaggtttcctgcaaggcatctggatacaccttcaccaattacaatatgcactgggtgcgacaggcccctggacaacggcttgagtggatgggaactatttatccaggaaatgatgatacttcctacaatcagaagttcaaagacagggtcacaatcactgcagacacatccgcgagcacagcctacatggagctgagcagcctgagatctgaggacacggccgtgtattactgtgctagagggggttatcgtgctatggactactggggtcaaggaaccctggtcaccgtctcctca

SEQ ID NO: 39 - DNA, 336 bp, Synthetic construct
  source 1..336: mol_type = other DNA
gatatcgtgatgactcagtctccactctccctgcccgtcaccccaggagagccggcctccatctcctgcagatctagtcagagcattgtatatagtaatggaaacacctatttaggatggtacctgcagaagccaggccaatctccacagctcctgatctacaaagtttccaaccgattttctggggtcccagacagattcagcggcagtgggtcaggcactgatttcacactgaaaatcagcagggtggaggctgaggatgttggggtttattactgctttcaaggttcacatgttccgtacacgttcggacaggggaccaagctggaaataaaa

SEQ ID NO: 40 - DNA, 1341 bp, Synthetic construct
  source 1..1341: mol_type = other DNA
caggtgcagctggtgcagtctggggctgaggtgaagaagcctggggcctcagtgaaggtttcctgcaaggcatctggatacaccttcaccaattacaatatgcactgggtgcgacaggcccctggacaacggcttgagtggatgggaactatttatccaggaaatgatgatacttcctacaatcagaagttcaaagacagggtcacaatcactgcagacacatccgcgagcacagcctacatggagctgagcagcctgagatctgaggacacggccgtgtattactgtgctagagggggttatcgtgctatggactactggggtcaaggaaccctggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggcgggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccactccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 41 - DNA, 651 bp, Synthetic construct
  source 1..651: mol_type = other DNA
gatatcgtgatgactcagtctccactctccctgcccgtcaccccaggagagccggcctccatctcctgcagatctagtcagagcattgtatatagtaatggaaacacctatttaggatggtacctgcagaagccaggccaatctccacagctcctgatctacaaagtttccaaccgattttctggggtcccagacagattcagcggcagtgggtcaggcactgatttcacactgaaaatcagcagggtggaggctgaggatgttggggtttattactgctttcaaggttcacatgttccgtacacgttcggacaggggaccaagctggaaataaaagtcgacgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 42 - DNA, 24 bp, Synthetic construct
  source 1..24: mol_type = other DNA
ggttattcattcactgactactgg

SEQ ID NO: 43 - DNA, 24 bp, Synthetic construct
  source 1..24: mol_type = other DNA
atttatcctagcaatactgttact

SEQ ID NO: 44 - DNA, 24 bp, Synthetic construct
  source 1..24: mol_type = other DNA
acaagattggggaattcggggtac

SEQ ID NO: 45 - DNA, 18 bp, Synthetic construct
  source 1..18: mol_type = other DNA
gaaagtgtcagttcacat

SEQ ID NO: 46 - DNA, 15 bp, Synthetic construct
  source 1..15: mol_type = other DNA
tataaagcatcaaac

SEQ ID NO: 47 - DNA, 27 bp, Synthetic construct
  source 1..27: mol_type = other DNA
cagcagggttggaatggtccgttcacg

SEQ ID NO: 48 - DNA, 372 bp, Synthetic construct
  source 1..372: mol_type = other DNA
caggtcaagctgctgcagtctggggctgcactggtgaagcctggagcctctgtgaagatgtcttgccaagcctctggttattcattcactgactactgggtgacctgggtgaagcagagtcatggacagagccttgagtggattggggaaatttatcctagcaatactgttactaacttcaatgataacttcaagggcaaggccacattgactgtagacaaatccaccagcacagcctatatggagctcagcagattgacatctgaggactctgcaatctattactgtacaagattggggaattcggggtacagagttggttggtttctttactggggccaaggcactctggtcactgtcatttcagcgtcg

SEQ ID NO: 49 - DNA, 333 bp, Synthetic construct
  source 1..333: mol_type = other DNA
tccgacactgtgctgacccagtctcctgctttggctgtgtctcctggagagagggttaccatctcctgtaaggccagtgaaagtgtcagttcacatatgcactggtaccaacagaaaccaggacagcaacccaaactcctcatctataaagcatcaaacctagcatctggggtccctgccaggttcagtggcagtgggtctgggacagacttcaccctcaccattgatcctgtggaggctgatgacactgcaacctacttctgtcagcagggttggaatggtccgttcacgtttggagctgggaccaggctggaactgaaacgggctgtcgac

SEQ ID NO: 50 - DNA, 1356 bp, Synthetic construct
  source 1..1356: mol_type = other DNA
caggtcaagctgctgcagtctggggctgcactggtgaagcctggagcctctgtgaagatgtcttgccaagcctctggttattcattcactgactactgggtgacctgggtgaagcagagtcatggacagagccttgagtggattggggaaatttatcctagcaatactgttactaacttcaatgataacttcaagggcaaggccacattgactgtagacaaatccaccagcacagcctatatggagctcagcagattgacatctgaggactctgcaatctattactgtacaagattggggaattcggggtacagagttggttggtttctttactggggccaaggcactctggtcactgtcatttcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggcgggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccactccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 51 - DNA, 642 bp, Synthetic construct
  source 1..642: mol_type = other DNA
tccgacactgtgctgacccagtctcctgctttggctgtgtctcctggagagagggttaccatctcctgtaaggccagtgaaagtgtcagttcacatatgcactggtaccaacagaaaccaggacagcaacccaaactcctcatctataaagcatcaaacctagcatctggggtccctgccaggttcagtggcagtgggtctgggacagacttcaccctcaccattgatcctgtggaggctgatgacactgcaacctacttctgtcagcagggttggaatggtccgttcacgtttggagctgggaccaggctggaactgaaacgggctgtcgacgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt